(19) United States
(12) Patent Application Publication
(10) Pub. No.: US 2012/0129849 A1
(43) Pub. Date: May 24, 2012

(54) DEUTERATED SERINE-THREONINE PROTEIN KINASE MODULATORS

(75) Inventors: Mustapha HADDACH, San Diego, CA (US); David M. Ryckman, San Diego, CA (US)

(73) Assignee: CYLENE PHARMACEUTICALS, INC., San Diego, CA (US)

(21) Appl. No.: 13/242,525

(22) Filed: Sep. 23, 2011

Related U.S. Application Data
(60) Provisional application No. 61/405,815, filed on Oct. 22, 2010.

(51) Int. Cl. A61K 31/473; C07D 471/04; A61K 31/4745; A61K 31/519; A61K 31/5377; C12N 5/02; A61P 27/02; A61P 37/00; A61P 31/00; A61P 29/00; A61P 25/00; C07D 221/12; A61P 35/00

(52) U.S. Cl. 514/232.8; 546/108; 546/88; 544/250; 544/115; 514/298; 514/292; 514/267; 435/375

(57) ABSTRACT

The present invention provides deuterated compounds having certain biological activities that include, but are not limited to, inhibiting cell proliferation, modulating protein kinase activity and modulating polymerase activity. The deuterated compounds of the invention can modulate casein kinase (CK) activity and/or poly(ADP-ribose)polymerase (PARP) activity. The invention also relates in part to methods for using such deuterated compounds as therapeutic agents.

### CROSS-REFERENCE TO RELATED APPLICATIONS

This application claims the benefit of priority of U.S. Provisional Application No. 61/405,815, filed Oct. 22, 2010 and entitled “DEUTERATED SERINE-THREONINE PROTEIN KINASE MODULATORS”. This application is also related to U.S. Utility application Ser. No. 11/849,230, filed on Aug. 31, 2007 and published as US 2009/0105233 A1 on Apr. 23, 2009, which claims priority under 35 U.S.C. §119(e) to U.S. Provisional Application Ser. No. 60/842,061 filed Sep. 1, 2006; U.S. Provisional Application Ser. No. 60/844,542 filed Sep. 13, 2006; U.S. Provisional Application Ser. No. 60/846,683 filed Sep. 22, 2006; U.S. Provisional Application Ser. No. 60/873,936 filed Dec. 7, 2006; and U.S. Provisional Application Ser. No. 60/859,716 filed Mar. 19, 2007. The contents of the aforementioned documents are incorporated herein by reference in their entirety for all purposes.

### FIELD OF THE INVENTION

The invention relates in part to deuterated compounds having certain biological activities that include, but are not limited to, inhibiting cell proliferation, modulating serine-threonine protein kinase activity. The compounds of the invention can modulate casein kinase (CK) activity (e.g., CK2 activity). The invention also relates in part to methods for using such compounds.

### BACKGROUND OF THE INVENTION

Serine-threonine protein kinases phosphorylate the hydroxyl group of serine or threonine. Because these protein kinases have important functions in biochemical pathways associated with cancer, immunological responses, inflammation, etc., and are also important in pathogenicity of certain microorganisms, modulators of their activity have many medicinal applications.

The casein kinase family of protein kinases, including I and II, are serine/threonine-selected enzymes. Protein kinase CK2 (formerly called Casein kinase II, referred to herein as “CK2”) is a ubiquitous and highly conserved protein serine/threonine kinase. The holoenzyme is typically found in tetrameric complexes consisting of two catalytic (alpha and/or alpha′) subunits and two regulatory (beta) subunits. CK2 has a number of physiological targets and participates in a complex series of cellular functions including the maintenance of cell viability. The level of CK2 in normal cells is tightly regulated, and it has long been considered to play a role in cell growth and proliferation. Inhibitors of CK2 that described as are useful for treating certain types of cancers are described in PCT/US2007/077464, PCT/US2008/074820, PCT/US2009/35609.

Both the prevalence and the importance of CK2 suggest it is an ancient enzyme on the evolutionary scale, as does an evolutionary analysis of its sequence; its longevity may explain why it has become important in so many biochemical processes, and why CK2 from hosts have even been co-opted by infectious pathogens (e.g., viruses, protozoa) as an integral part of their survival and life cycle biochemical systems. These same characteristics explain why inhibitors of CK2 are believed to be useful in a variety of medical treatments as discussed herein. Because it is central to many biological processes, as summarized by Guerra & Issinger, Curr. Med. Chem., 2008, 15:1870-1886, inhibitors of CK2, including the compounds described herein, should be useful in the treatment of a variety of diseases and disorders. Cancerous cells show an elevation of CK2, and recent evidence suggests that CK2 exerts potent suppression of apoptosis in cells by protecting regulatory proteins from caspase-mediated degradation. The anti-apoptotic function of CK2 may contribute to its ability to participate in transformation and tumorigenesis. In particular, CK2 has been shown to be associated with acute and chronic myelogenous leukemia, lymphoma and multiple myeloma. In addition, enhanced CK2 activity has been observed in solid tumors of the colon, rectum and breast, squamous cell carcinomas of the lung and of the head and neck (SCCHN), adenocarcinomas of the lung, colon, rectum, kidney, breast, and prostate. Inhibition of CK2 by a small molecule is reported to induce apoptosis of pancreatic cancer cells, and hepatocellular carcinoma cells (HegG2, Hep3, HeLa cancer cell lines); and CK2 inhibitors dramatically sensitized RMS (Rhabdomyosarcoma) tumors toward apoptosis induced by TRAIL. Thus an inhibitor of CK2 alone, or in combination with TRAIL or a ligand for the TRAIL receptor, would be useful to treat RMS, the most common soft-tissue sarcoma in children. In addition, elevated CK2 has been found to be highly correlated with aggressiveness of neoplasias, and treatment with a CK2 inhibitor of the invention should thus reduce tendency of benign lesions to advance into malignant ones, or for malignant ones to metastasize.

Unlike other kinases and signaling pathways, where mutations are often associated with structural changes that cause loss of regulatory control, increased CK2 activity level appears to be generally caused by upregulation or overexpression of the active protein rather than by changes that affect activation levels. Guerra and Issinger postulate this may be due to regulation by aggregation, since activity levels do not correlate well with mRNA levels. Excessive activity of CK2 has been shown in many cancers, including SCCHN tumors, lung tumors, breast tumors, and others. Id.

Elevated CK2 activity in colorectal carcinomas was shown to correlate with increased malignancy. Aberrant expression and activity of CK2 have been reported to promote increase nuclear levels of NF-kappaB in breast cancer cells. CK2 activity is markedly increased in patients with AML and CML during blast crisis, indicating that an inhibitor of CK2 should be particularly effective in these conditions. Multiple myeloma cell survival has been shown to rely on high activity of CK2, and inhibitors of CK2 were cytotoxic to MM cells. Similarly, a CK2 inhibitor inhibited growth of murine p190 lymphoma cells. Its interaction with Bcr/Abl has been reported to play an important role in proliferation of Bcr/Abl expressing cells, indicating inhibitors of CK2 may be useful in treatment of Bcr/Abl-positive leukemias. Inhibitors of CK2 have been shown to inhibit progression of skin papillomas, prostate and breast cancer xenografts in mice, and to prolong survival of transgenic mice that express prostate-promoters. Id.

The role of CK2 in various non-cancer disease processes has been recently reviewed. See Guerra & Issinger, Curr. Med. Chem., 2008, 15:1870-1886. Increasing evidence indicates that CK2 is involved in critical diseases of the central nervous system, including, for example, Alzheimer's disease, Parkinson's disease, and rare neurodegenerative disorders such as Guam-Parkinson dementia, chromosome 18 deletion syndrome, progressive supranuclear palsy, Kuf'disease, or Pick's disease. It is suggested that selective CK2-mediated phosphorylation of tau proteins may be involved in progressive neurodegeneration of Alzheimer's. In addition, recent studies suggest that CK2 plays a role in memory impairment and brain ischemia, the latter effect apparently being mediated by CK2′s regulatory effect on the PI3K survival pathways.

CK2 has also been shown to be involved in the modulation of inflammatory disorders, for example, acute or chronic inflammatory pain, glomerulonephritis, and autoimmune diseases, including, e.g., multiple sclerosis (MS), systemic lupus erythematosus, rheumatoid arthritis, and juvenile arthritis. It positively regulates the function of the serotonin 5-HT3 receptor channel, activates heme oxygenase type 2, and enhances the activity of neuronal nitric oxide synthase. A selective CK2 inhibitor was reported to strongly reduce pain response of mice when administered to spinal cord tissue prior to pain testing. It phosphorylates secretory type IIA phospholipase A2 from synovial fluid of RA patients, and modulates secretion of DEK (a nuclear DNA-binding protein), which is a proinflammatory molecule found in synovial fluid of patients with juvenile arthritis. Thus inhibition of CK2 is expected to control progression of inflammatory pathologies such as those described here, and the inhibitors disclosed herein have been shown to effectively treat pain in animal models.

Protein kinase CK2 has also been shown to play a role in disorders of the vascular system, such as, e.g., atherosclerosis, laminar shear stress, and hypoxia. CK2 has also been shown to play a role in disorders of skeletal muscle and bone tissue, such as cardiomyocyte hypertrophy, impaired insulin signaling and bone tissue mineralization. In one study, inhibitors of CK2 were effective at slowing angiogenesis induced by growth factor in cultured cells. Moreover, in a retinopathy model, a CK2 inhibitor combined with octreotide (a somatostatin analog) reduced neovascular tufts; thus the CK2 inhibitors described herein would be effective in combination with a somatostatin analog to treat retinopathy.

CK2 has also been shown to phosphorylate GSK, troponin and myosin light chain; thus it is important in skeletal muscle and bone tissue physiology, and is linked to diseases affecting muscle tissue.

Evidence suggests that CK2 is also involved in the development and life cycle regulation of protozoal parasites, such as, for example, Theileria parva, Trypanosoma cruzi, Leishmania donovani, Herpetomonas muscarum muscarum, Plasmodium falciparum, Trypanosoma brucei, Toxoplasma gondii and Schistosoma mansoni. Numerous studies have confirmed the role of CK2 in regulation of cellular motility of protozoan parasites, essential to invasion of host cells. Activation of CK2 or excessive activity of CK2 has been shown to occur in hosts infected with Leishmania donovani, Herpetomonas muscarum muscarum, Plasmodium falciparum, Trypanosoma brucei, Toxoplasma gondii and Schistosoma mansoni. Indeed, inhibition of CK2 has been shown to block infection by T. cruzi.

CK2 has also been shown to interact with and/or phosphorylate viral proteins associated with human immunodeficiency virus type 1 (HIV-1), human papilloma virus, and herpes simplex virus, in addition to other virus types (e.g. human cytomegalovirus, hepatitis C and B viruses, Borna disease virus, adenovirus, coxsackievirus, coronavirus, influenza, and varicella zoster virus). CK2 phosphorylates and activates HIV-1 reverse transcriptase and proteases in vitro and in vivo, and promotes pathogenicity of simian-human immunodeficiency virus (SHIV), a model for HIV. Inhibitors of CK2 are thus able to reduce reduce pathogenic effects of a model of HIV infection. CK2 also phosphorylates numerous proteins in herpes simplex virus and numerous other viruses, and some evidence suggests viruses have adopted CK2 as a phosphorylating enzyme for their essential life cycle proteins. Inhibition of CK2 is thus expected to deter infection and progression of viral infections, which rely upon the host's CK2 for their own life cycles.

CK2 is unusual in the diversity of biological processes that it affects, and it differs from most kinases in other ways as well: it is constitutively active, it can use ATP or GTP, and it is elevated in most tumors and rapidly proliferating tissues. It also has unusual structural features that may distinguish it from most kinases, too, enabling its inhibitors to be highly specific for CK2 while many kinase inhibitors affect multiple kinases, increasing the likelihood of off-target effects, or variability between individual subjects. For all of these reasons, CK2 is a particularly interesting target for drug development, and effective inhibitors of CK2 can be useful in treating a variety of different diseases and disorders mediated by or associated with excessive, aberrant or undesired levels of CK2 activity.

There is a need in the art for the development of compounds having favorable drug-like features and being capable of modulating serine-threonine protein kinase (such as CK2) activity for use in the treatment of related conditions or diseases.

### SUMMARY OF THE INVENTION

In one embodiment, the present invention provides a compound having structural Formula (A):

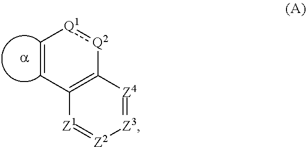

or a pharmaceutically acceptable salt, solvate, and/or prodrug thereof;
wherein the ring labeled α represents a 5 or 6 membered aromatic or heteroaromatic ring fused onto the ring containing Q¹, wherein a is a 6-membered aryl ring optionally containing one or more nitrogen atoms as ring members, or a 5-membered aryl ring selected from thiophene and thiazole; and the ring labeled α optionally contains one or more carbon-bound deuterium;
   Q¹ is C═X, Q² is NR^{S}, and the bond between Q¹ and Q² is a single bond; or Q¹ is C—X—R⁵, Q² is N, and the bond between Q¹ and Q² is a double bond; and
   wherein X represents O, S or NR⁴;
each Z¹, Z², Z³, and Z⁴ is N or CR³ and one or more of Z¹, Z², Z³, and Z⁴ is CR³;
   each R³ is independently H, deuterium, or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C12 heteroaryl, deuterated-C7-C12 arylalkyl, or deuterated-C6-C12 heteroarylalkyl group,
   or each R³ can be halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
      wherein each R is independently H, deuterium, C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C10 heteroaryl, deuterated-C7-C12 arylalkyl, or deuterated-C6-C12 heteroarylalkyl,
         and wherein two R on the same atom or on adjacent atoms can be linked to form a 3 to 8 membered ring, optionally containing one or more N, O or S; and the 3 to 8 membered ring optionally contains one or more carbon-bound deuterium;
         and each R group, and each ring formed by linking two R groups together, is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
            wherein each R′ is independently H, deuterium, C1-C6 alkyl, C2-C6 heteroalkyl, C1-C6 acyl, C2-C6 heteroacyl, C6-C10 aryl, C5-C10 heteroaryl, C7-12 arylalkyl, C6-12 heteroarylalkyl, deuterated-C1-C6 alkyl, deuterated-C2-C6 heteroalkyl, deuterated-C1-C6 acyl, deuterated-C2-C6 heteroacyl, deuterated-C6-C10 aryl, deuterated-C5-C10 heteroaryl, deuterated-C7-12 arylalkyl, or deuterated-C6-12 heteroarylalkyl; each of which is optionally substituted with one or more groups selected from halo, C1-C4 alkyl, C1-C4 heteroalkyl, C1-C6 acyl, C1-C6 heteroacyl, hydroxy, amino, deuterated-C1-C4 alkyl, deuterated-C1-C4 heteroalkyl, deuterated-C1-C6 acyl, deuterated-C1-C6 heteroacyl, deuterated-hydroxy, deuterated-amino, and ═O;
         and wherein two R′ can be linked to form a 3 to 7 membered ring optionally containing up to three heteroatoms selected from N, O and S; and the 3 to 7 membered ring optionally contains one or more carbon-bound deuterium;
R⁴ is H, deuterium, or optionally substituted member selected from the group consisting of C₁-C₆ alkyl, C2-C6 heteroalkyl, C1-C6 acyl, deuterated-C₁-C₆ alkyl, deuterated-C2-C6 heteroalkyl, and deuterated-C1-C6 acyl;
each R⁵ is independently H, deuterium, or an optionally substituted member selected from the group consisting of C₁₋₁₀ alkyl, C₂₋₁₀ alkenyl, C₂₋₁₀ heteroalkyl, C₃₋₈ carbocyclic ring, C₃₋₈ heterocyclic ring, deuterated-C₁₋₁₀ alkyl, deuterated-C₂₋₁₀ alkenyl, deuterated-C₂₋₁₀ heteroalkyl, deuterated-C₃₋₈ carbocyclic ring, and deuterated-C₃₋₈ heterocyclic ring optionally fused to an additional optionally substituted carbocyclic, heterocyclic, deuterated-carbocyclic, deuterated-heterocyclic ring; or R⁵ is a C₁₋₁₀ alkyl, C₂₋₁₀ alkenyl, C₂₋₁₀ heteroalkyl, deuterated-C₁₋₁₀ alkyl, deuterated-C₂₋₁₀ alkenyl, or deuterated-C₂₋₁₀ heteroalkyl substituted with an optionally substituted C₃₋₈ carbocyclic ring, C₃₋₈ heterocyclic ring, deuterated-C₃₋₈ carbocyclic ring, or deuterated-C₃₋₈ heterocyclic ring; and
   in each —NR⁴R⁵, R⁴ and R⁵ together with N may form an optionally substituted 3 to 8 membered ring, which may optionally contain an additional heteroatom selected from N, O and S as a ring member; and the 3 to 8 membered ring optionally contains one or more carbon-bound deuterium; and
with the following provisos:
   (a) the compound of Formula (A) comprises at least one carbon-bound deuterium; and
   (b) when Q¹ in Formula (A) is C—NHΦ, where Φ is optionally substituted phenyl:
      if the ring labeled α is a six-membered ring containing at least one N as a ring member, at least one R³ present must be a polar substituent, or if each R³ is H, then (I) must be substituted; and
      if the ring labeled α is phenyl, and three of Z¹ to Z⁴ represent CH, then Z² cannot be C—OR″, and Z³ cannot be NH₂, NO₂, NHC(═O)R″ or NHC(═O)—OR″, where R″ is C1-C4 alkyl.

In another embodiment, the present invention provides a pharmaceutical composition comprising a compound as described above, or a pharmaceutically acceptable salt, solvate, and/or prodrug thereof; and a pharmaceutically acceptable carrier.

In another embodiment, the present invention provides a method of modulating a serine-threonine protein kinase activity in a cell, comprising contacting the cell with a compound as described above, or a pharmaceutically acceptable salt, solvate, and/or prodrug thereof in an amount effective to modulate a serine-threonine protein kinase activity.

In another embodiment, the present invention provides a method of inhibiting cell proliferation, comprising contacting cells with a compound as described above, or a pharmaceutically acceptable salt, solvate, and/or prodrug thereof in an amount effective to inhibit proliferation of the cells.

In another embodiment, the present invention provides a method of treating a condition or disease related to aberrant cell proliferation, comprising administering a therapeutically effective amount of a compound as described above, or a pharmaceutically acceptable salt, solvate, and/or prodrug thereof, to a subject in need thereof.

In another embodiment, the present invention provides a method of treating a condition or disease associated with a serine-threonine protein kinase activity, comprising administering a therapeutically effective amount of a compound as described above, or a pharmaceutically acceptable salt, solvate, and/or prodrug thereof, to a subject in need thereof.

In another embodiment, the present invention provides a method to treat a condition related to aberrant cell proliferation, which comprises co-administering to a subject in need of treatment for such condition a compound as described above, or a pharmaceutically acceptable salt, solvate, and/or prodrug thereof; and at least one additional therapeutic agent.

### DETAILED DESCRIPTION OF THE INVENTION

The present invention in part provides deuterated chemical compounds having certain biological activities that include, but are not limited to, inhibiting cell proliferation, inhibiting angiogenesis, and modulating protein kinase activity. These molecules can modulate serine-threonine protein kinase activity including casein kinase 2 (CK2) activity, and thus affect biological functions that include but are not limited to, inhibiting gamma phosphate transfer from ATP to a protein or peptide substrate, inhibiting angiogenesis, inhibiting cell proliferation and inducing cell apoptosis, for example. The present invention also in part provides methods for preparing those chemical compounds, and analogs thereof, and methods of using the foregoing. Also provided are compositions comprising the above-described compounds in combination with other agents, and methods for using such compounds in combination with other agents.

With respect to small molecules, such as chemical compounds, deuterium-substitution is one of many approaches to provide variations of compounds potentially useful for therapeutic treatment. General exposure to and incorporation of deuterium is safe within levels potentially achieved by use of compounds of this invention as medicaments. For instance, the weight percentage of hydrogen in a mammal (approximately 9%) and natural abundance of deuterium (approximately 0.015%) indicates that a 70 kg human normally contains nearly a gram of deuterium. Furthermore, replacement of up to about 15% of normal hydrogen with deuterium has been effected and maintained for a period of days to weeks in mammals, including rodents and dogs, with minimal observed adverse effects. Although higher deuterium concentrations, usually in excess of 20%, may be toxic in animals, acute replacement of as high as 15% to 23% of the hydrogen in humans' fluids with deuterium has been found to not cause toxicity. In a 70 kg human male, 15% replacement of the hydrogen in the fluid compartment with deuterium corresponds to incorporation of approximately 1 kg of deuterium or the equivalent of approximately 5 kg of deuterated water. Deuterium tracers, such as deuterium-labeled drugs and doses, in some cases repeatedly, of thousands of milligrams of deuterated water, are also used in healthy humans of all ages, including neonates and pregnant women, without reported incident. Thus, it is clear that any deuterium released, for instance, during the metabolism of compounds of this invention poses no health risk.

Additional embodiments and advantages of the application will be set forth in part in the description that follows, and in part will be obvious from the description, or may be learned by practice of the invention. It is to be understood that both the foregoing general description and the following detailed description are exemplary and explanatory only and are not restrictive of the invention as claimed.

### Embodiments of Compounds

One embodiment of the invention is the compound having the structural Formula (A):

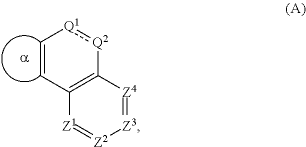

or a pharmaceutically acceptable salt, solvate, and/or prodrug thereof;
wherein the ring labeled α represents a 5 or 6 membered aromatic or heteroaromatic ring fused onto the ring containing Q¹, wherein a is a 6-membered aryl ring optionally containing one or more nitrogen atoms as ring members, or a 5-membered aryl ring selected from thiophene and thiazole; and the ring labeled α optionally contains one or more carbon-bound deuterium;
   Q¹ is C═X, Q² is NR⁵, and the bond between Q¹ and Q² is a single bond; or Q¹ is C—X—R⁵, Q² is N, and the bond between Q¹ and Q² is a double bond; and
   wherein X represents O, S or NR⁴;
each Z¹, Z², Z³, and Z⁴ is N or CR³ and one or more of Z¹, Z², Z³, and Z⁴ is CR³;
   each R³ is independently H, deuterium, or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C12 heteroaryl, deuterated-C7-C12 arylalkyl, or deuterated-C6-C12 heteroarylalkyl group,
      or each R³ can be halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
         wherein each R is independently H, deuterium, C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C10 heteroaryl, deuterated-C7-C12 arylalkyl, or deuterated-C6-C12 heteroarylalkyl,
            and wherein two R on the same atom or on adjacent atoms can be linked to form a 3 to 8 membered ring, optionally containing one or more N, O or S; and the 3 to 8 membered ring optionally contains one or more carbon-bound deuterium;
            and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
            wherein each R′ is independently H, deuterium, C1-C6 alkyl, C2-C6 heteroalkyl, C1-C6 acyl, C2-C6 heteroacyl, C6-C10 aryl, C5-C10 heteroaryl, C7-12 arylalkyl, C6-12 heteroarylalkyl, deuterated-C1-C6 alkyl, deuterated-C2-C6 heteroalkyl, deuterated-C1-C6 acyl, deuterated-C2-C6 heteroacyl, deuterated-C6-C10 aryl, deuterated-C5-C10 heteroaryl, deuterated-C7-12 arylalkyl, or deuterated-C6-12 heteroarylalkyl; each of which is optionally substituted with one or more groups selected from halo, C1-C4 alkyl, C1-C4 heteroalkyl, C1-C6 acyl, C1-C6 heteroacyl, hydroxy, amino, deuterated-C1-C4 alkyl, deuterated-C1-C4 heteroalkyl, deuterated-C1-C6 acyl, deuterated-C1-C6 heteroacyl, deuterated-hydroxy, deuterated-amino, and ═O;
            and wherein two R′ can be linked to form a 3 to 7 membered ring optionally containing up to three heteroatoms selected from N, O and S; and the 3 to 7 membered ring optionally contains one or more carbon-bound deuterium;
R⁴ is H, deuterium, or optionally substituted member selected from the group consisting of C₁-C₆ alkyl, C2-C6 heteroalkyl, C1-C6 acyl, deuterated-C₁-C₆ alkyl, deuterated-C2-C6 heteroalkyl, and deuterated-C1-C6 acyl;
each R⁵ is independently H, deuterium, or an optionally substituted member selected from the group consisting of C₁₋₁₀ alkyl, C₂₋₁₀ alkenyl, C₂₋₁₀ heteroalkyl, C₃₋₈ carbocyclic ring, C₃₋₈ heterocyclic ring, deuterated-C₁₋₁₀ alkyl, deuterated-C₂₋₁₀ alkenyl, deuterated-C₂₋₁₀ heteroalkyl, deuterated-C₃₋₈ carbocyclic ring, and deuterated-C₃₋₈ heterocyclic ring optionally fused to an additional optionally substituted carbocyclic, heterocyclic, deuterated-carbocyclic, deuterated-heterocyclic ring; or R⁵ is a C₁₋₁₀ alkyl, C₂₋₁₀ alkenyl, C₂₋₁₀ heteroalkyl, deuterated-C₁₋₁₀ alkyl, deuterated-C₂₋₁₀ alkenyl, or deuterated-C₂₋₁₀ heteroalkyl substituted with an optionally substituted C₃₋₈ carbocyclic ring, C₃₋₈ heterocyclic ring, deuterated-C₃₋₈ carbocyclic ring, or deuterated-C₃₋₈ heterocyclic ring; and
   in each —NR⁴R⁵, R⁴ and R⁵ together with N may form an optionally substituted 3 to 8 membered ring, which may optionally contain an additional heteroatom selected from N, O and S as a ring member; and the 3 to 8 membered ring optionally contains one or more carbon-bound deuterium; and
with the following provisos:
   (a) the compound of Formula (A) comprises at least one carbon-bound deuterium; and
   (b) when Q¹ in Formula (A) is C—NHΦ, where φ is optionally substituted phenyl:
      if the ring labeled α is a six-membered ring containing at least one N as a ring member, at least one R³ present must be a polar substituent, or if each R³ is H, then Φ must be substituted; and
      if the ring labeled α is phenyl, and three of Z¹ to Z⁴ represent CH, then Z² cannot be C—OR″, and Z³ cannot be NH₂, NO₂, NHC(═O)R″ or NHC(═O)—OR″, where R″ is C1-C4 alkyl.

Yet another embodiment of the invention is a compound having a structural Formula I, II, III or IV:

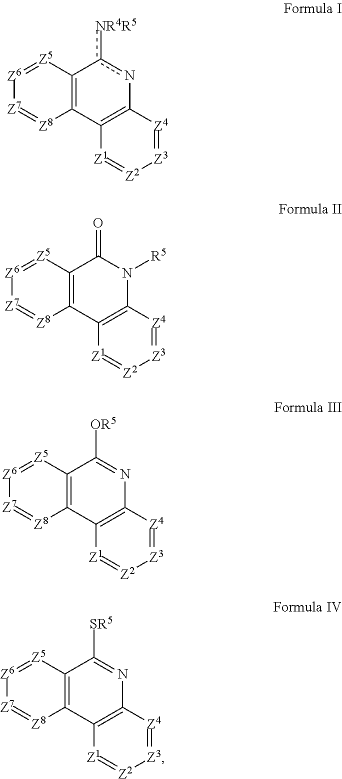

or a pharmaceutically acceptable salt, solvate, and/or prodrug thereof;
   wherein:
each Z¹, Z², Z³, and Z⁴ is N or CR³;
each of Z⁵, Z⁶, Z⁷ and Z⁸ is N or CR⁶;
none, one or two of Z¹ to Z⁴ are N and none, one or two of Z⁵-Z⁸ are N;
   each R³ and each R⁶ is independently H, deuterium, or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C12 heteroaryl, deuterated-C7-C12 arylalkyl, or deuterated-C6-C12 heteroarylalkyl group,
   or each R³ and each R⁶ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, polar substituent, carboxy bioisostere, COOH, COOD, or NO₂,
      wherein each R is independently H, deuterium, or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C10 heteroaryl, deuterated-C7-C12 arylalkyl, or deuterated-C6-C12 heteroarylalkyl,
         and wherein two R on the same atom or on adjacent atoms can be linked to form a 3 to 8 membered ring, optionally containing one or more N, O or S; and the 3 to 8 membered ring optionally contains one or more carbon-bound deuterium;
         and each R group, and each ring formed by linking two R groups together, is optionally substituted with one or more substituents selected from halo, ═O, ═N—CN, ═N—OR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
            wherein each R′ is independently H, deuterium, C1-C6 alkyl, C2-C6 heteroalkyl, C1-C6 acyl, C2-C6 heteroacyl, C6-C10 aryl, C5-C10 heteroaryl, C7-12 arylalkyl, C6-12 heteroarylalkyl, deuterated-C1-C6 alkyl, deuterated-C2-C6 heteroalkyl, deuterated-C1-C6 acyl, deuterated-C2-C6 heteroacyl, deuterated-C6-C10 aryl, deuterated-C5-C10 heteroaryl, C7-12 arylalkyl, or deuterated-C6-12 heteroarylalkyl each of which is optionally substituted with one or more groups selected from halo, C1-C4 alkyl, C1-C4 heteroalkyl, C1-C6 acyl, C1-C6 heteroacyl, hydroxy, amino, deuterated-C1-C4 alkyl, deuterated-C1-C4 heteroalkyl, deuterated-C1-C6 acyl, deuterated-C1-C6 heteroacyl, deuterated-hydroxy, deuterated-amino, and ═O;
         and wherein two R′ can be linked to form a 3 to 7 membered ring optionally containing up to three heteroatoms selected from N, O and S; and the 3 to 7 membered ring optionally contains one or more carbon-bound deuterium;
R⁴ is H or an optionally substituted member selected from the group consisting of C1-C6 alkyl, C2-C6 heteroalkyl, C1-C6 acyl, deuterated-C1-C6 alkyl, deuterated-C2-C6 heteroalkyl, and deuterated-C1-C6 acyl;
each R⁵ is independently H or an optionally substituted member selected from the group consisting of C₁₋₁₀ alkyl, C₂₋₁₀ alkenyl, C₂₋₁₀ heteroalkyl, C₃₋₈ carbocyclic ring, C₃₋₈ heterocyclic ring, deuterated-C₁₋₁₀ alkyl, deuterated-C₂₋₁₀ alkenyl, deuterated-C₂₋₁₀ heteroalkyl, deuterated-C₃₋₈ carbocyclic ring, and deuterated-C₃₋₈ heterocyclic ring optionally fused to an additional optionally substituted carbocyclic, heterocyclic, deuterated-carbocyclic, or deuterated-heterocyclic ring; or R⁵ is a C₁₋₁₀ alkyl, C₂₋₁₀ alkenyl, C₂₋₁₀ heteroalkyl, deuterated-C₁₋₁₀ alkyl, deuterated-C₂₋₁₀ alkenyl, or deuterated-C₂₋₁₀ heteroalkyl substituted with an optionally substituted C₃₋₈ carbocyclic ring, deuterated-C₃₋₈ carbocyclic ring, C₃₋₈ heterocyclic ring, or deuterated-C₃₋₈ heterocyclic ring; and
   in each —NR⁴R⁵, R⁴ and R⁵ together with N may form an optionally substituted 3 to 8 membered ring, which may optionally contain an additional heteroatom selected from N, O and S as a ring member; and the 3 to 8 membered ring optionally contains one or more carbon-bound deuterium;
with the following provisos:
   (a) the compound of Formula I, II, III, or IV comprises at least one carbon-bound deuterium; and
   (b) when —NR⁴R⁵ in Formula (I) is —NHΦ, where Φ is optionally substituted phenyl:
      if all of Z⁵ to Z⁸ are CH or one of Z⁵ to Z⁸ is N, at least one of Z¹ to Z⁴ is CR³ and at least one R³ must be a non-hydrogen substituent; or
      if each R³ is H, then Φ must be substituted; or
      if all of Z⁵ to Z⁸ are CH or one of Z⁵ to Z⁸ is N, then Z² is not C—OR″, and Z³ is not NH₂, NO₂, NHC(═O)R″ or NHC(═O)—OR″, where R″ is C1-C4 alkyl.

In yet another embodiment, the invention is the compound having a structural Formula I, II, III, or IV as described above, wherein at least one of R³ or R⁶ is a polar substituent, wherein said polar substituent is a carboxylic acid, carboxylate salt, carboxylate ester, carboxamide, tetrazole, carboxy bioisostere, deuterated-carboxylic acid, deuterated-carboxylate salt, deuterated-carboxylate ester, deuterated-carboxamide, deuterated-tetrazole, or deuterated-carboxy bioisostere.

In a further embodiment the invention is the compound having a structural Formula I, II, III, or IV as described above,wherein at least one R³ is a polar substituent.

In yet another embodiment, the invention is the compound having a structural Formula A, I, II, III, or IV as described above, wherein the ring containing Z¹ to Z⁴ is selected from one of the following structures

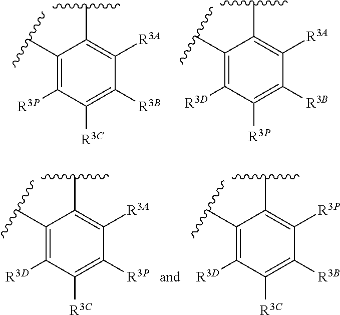

wherein R^{3P} is a polar substituent; and each R^{3A}, R^{3B}, R^{3C} and R^{3D} independently is selected from R³ substituents.

In a further embodiment, the invention is the above compound wherein each R^{3A}, R^{3C} and R^{3D} is H or deuterium; and R^{3B} is a polar substituent.

Another embodiment of the invention is any of the above compounds wherein at least one of Z¹ to Z⁴ and Z⁵ to Z⁸ is a nitrogen atom.

Another embodiment of the invention is any of the above compounds wherein R⁴ is H or deuterium.

Yet another embodiment of the invention is any of the compounds described above, wherein R⁵ is an optionally substituted 3 to 8 membered ring, and the 3 to 8 membered ring optionally contains one or more carbon-bound deuterium.

A further embodiment of the invention is any of the above compounds, wherein R⁵ is a C₁₋₁₀ alkyl or deuterated-C₁₋₁₀ alkyl group substituted with (1) an optionally substituted 3-8 membered ring, and the 3 to 8 membered ring optionally contains one or more carbon-bound deuterium; or (2) —NR⁴R⁵.

Another embodiment of the invention is the above compound, wherein R⁵ is a C₁₋₃ alkyl or deuterated-C₁₋₃ alkyl group substituted with (1) an optionally substituted phenyl, pyridyl, morpholino, deuterated-phenyl, deuterated-pyridyl or deuterated-morpholino ring substituent; or (2) substituted with —NR⁴R⁵.

Yet another embodiment of the invention is any of the compounds described above, wherein R⁵ is an optionally substituted six-membered carbocyclic, heterocyclic, deuterated-carbocyclic, or deuterated-heterocyclic ring.

Another embodiment of the invention is the above compound, wherein R⁵ is an optionally substituted phenyl or deuterated-phenyl ring.

A further embodiment of the invention is the above compound, wherein the compound has a structure of Formula I, R⁴ is H, deuterium, CD₃, CHD₂, CH_{I}D, or CH₃; and R⁵ is a phenyl or deuterated-phenyl substituted with one or more halogen or acetylene substituents.

In yet another embodiment of the invention is the above compound, wherein the one or more halogen or acetylene substituents are on the phenyl or deuterated-phenyl ring at the 3-position, 4-position or 5-position, or combinations thereof.

A further embodiment of the invention is any of the above compounds, wherein the R⁶ substituent is a —NR⁴R⁵ substituent.

Another embodiment of the invention is the above compound, wherein the R⁶ substituent is a —NH—(C1-C6 alkyl), —ND-(C1-C6 alkyl), —NH-(deuterated-C1-C6 alkyl), —ND-(deuterated-C1-C6 alkyl), —NH—(C3-C8 cycloalkyl), —ND-(C3-C8 cycloalkyl), —NH-(deuterated-C3-C8 cycloalkyl), —ND-(deuterated-C3-C8 cycloalkyl) moiety.

Yet another embodiment of the invention is the compound having a structural Formulae Ia, Ib, Ic, or Id:

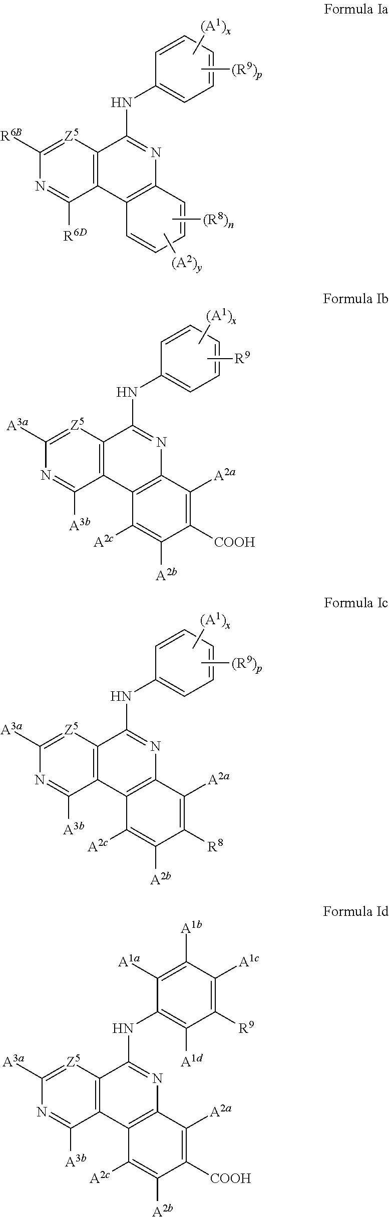

or a pharmaceutically acceptable salt, solvate, and/or prodrug thereof; wherein: Z⁵ is N or CR^{6A};
each R^{6A}, R^{6B}, R^{6C} and R⁸ independently is H, deuterium, or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C12 heteroaryl, deuterated-C7-C12 arylalkyl, or deuterated-C6-C12 heteroarylalkyl group,
or each R^{6A}, R^{6B}, R^{6C} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,
each A¹, A^{1a}, A^{1b}, A^{1c}, A^{1d}, A², A^{2a}, A^{2b}, A^{2c}, A^{3a}, and A^{3b} is independently H or deuterium;
R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C12 heteroaryl, deuterated-C7-C12 arylalkyl, or deuterated-C6-C12 heteroarylalkyl group, or
R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H, deuterium, C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C10 heteroaryl, deuterated-C7-C12 arylalkyl, or deuterated-C6-C12 heteroarylalkyl;
and wherein two R on the same atom or on adjacent atoms can be linked to form a 3 to 8 membered ring, optionally containing one or more N, O or S; and the 3 to 8 membered ring contains one or more carbon-bound deuterium;
and each R group, and each ring formed by linking two R groups together, is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, deuterium, C1-C6 alkyl, C2-C6 heteroalkyl, C1-C6 acyl, C2-C6 heteroacyl, C6-C10 aryl, C5-C10 heteroaryl, C7-12 arylalkyl, C6-12 heteroarylalkyl, deuterated-C1-C6 alkyl, deuterated-C2-C6 heteroalkyl, deuterated-C1-C6 acyl, deuterated-C2-C6 heteroacyl, deuterated-C6-C10 aryl, deuterated-C5-C10 heteroaryl, deuterated-C7-12 arylalkyl, or deuterated-C6-12 heteroarylalkyl each of which is optionally substituted with one or more groups selected from halo, C1-C4 alkyl, C1-C4 heteroalkyl, C1-C6 acyl, C1-C6 heteroacyl, hydroxy, amino, deuterated-C1-C4 alkyl, deuterated-C1-C4 heteroalkyl, deuterated-C1-C6 acyl, deuterated-C1-C6 heteroacyl, deuterated-hydroxy, deuterated-amino, and ═O;

and wherein two R′ can be linked to form a 3 to 7 membered ring optionally containing up to three heteroatoms selected from N, O and S; and the 3 to 7 membered ring contains one or more carbon-bound deuterium;
x is 1 to 5;
y is 0 to 4;
n is 0 to 4; and
p is 0 to 4; and
with the following provisos:
   (a) the compound of Formula Ia, Ib, Ic, or Id comprises at least one carbon-bound deuterium; and
   (b) x plus p is 5, and y plus n is 4.

Another embodiment of the invention is the compound having a structural Formulae Ia, Ib, Ic, or Id as described above, wherein Z⁵ is N.

A further embodiment of the invention is the compound having a structural Formulae Ia, Ib, Ic, or Id as described above, wherein R⁸ is a caboxy moiety, deuterated-caboxy moiety, carboxy bioisostere, or deuterated-carboxy bioisostere.

Another embodiment of the invention is the above compound wherein the carboxy or deuterated-carboxy moiety is a carboxylate, deuterated-carboxylate, carboxylic acid, or deuterated-carboxylic acid.

Another embodiment of the invention is the compound having a structural Formulae Ia, Ib, Ic, or Id as described above, wherein R⁹ is selected from —C≡CR, —C≡CH, —C≡CD, methyl, deuterated-methyl, ethyl, deuterated-ethyl, —CF₃, —C≡N, —OR and halogen.

In certain specific embodiments, the compound of the invention has one of the following structures in a deuterated-form:

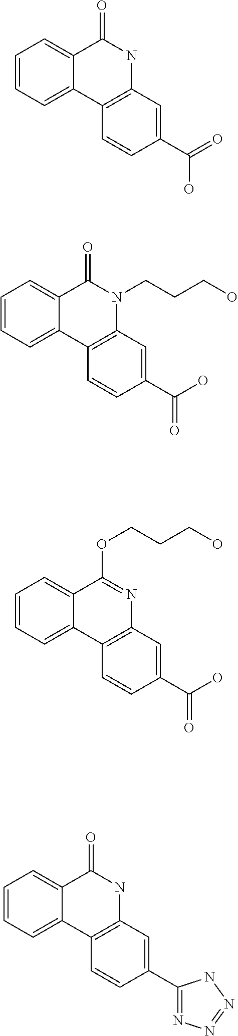

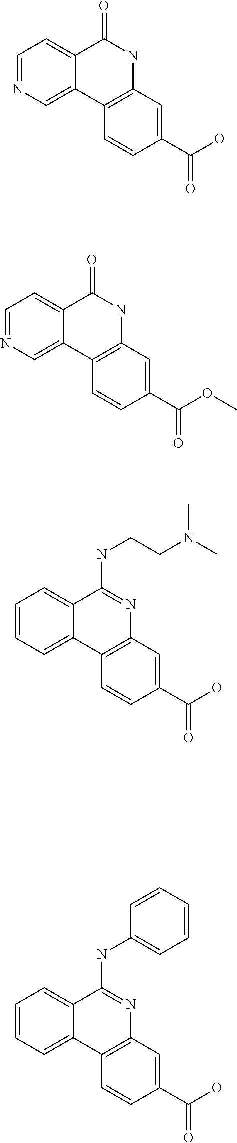

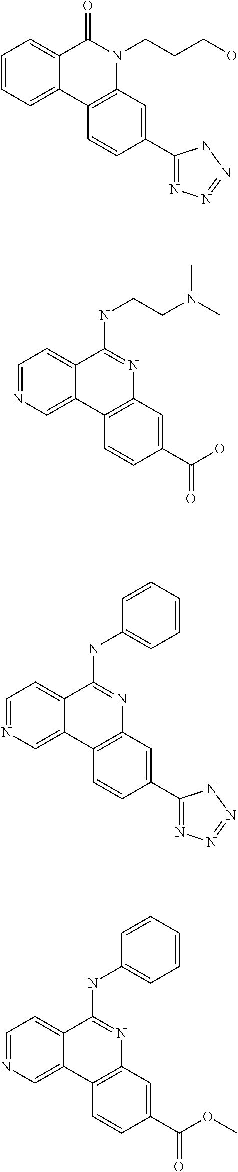

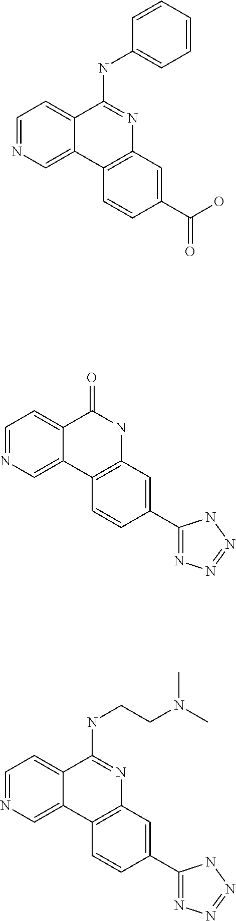

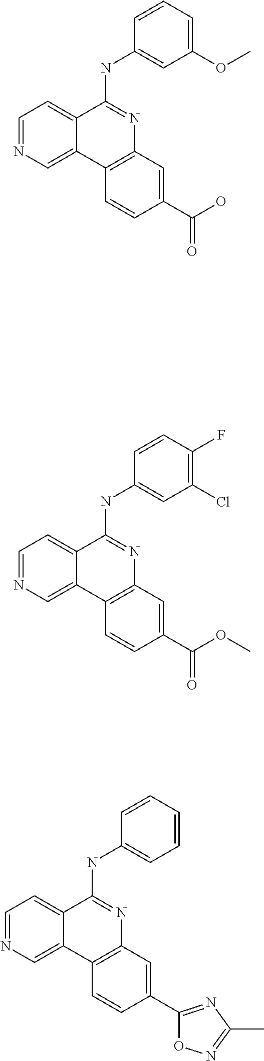

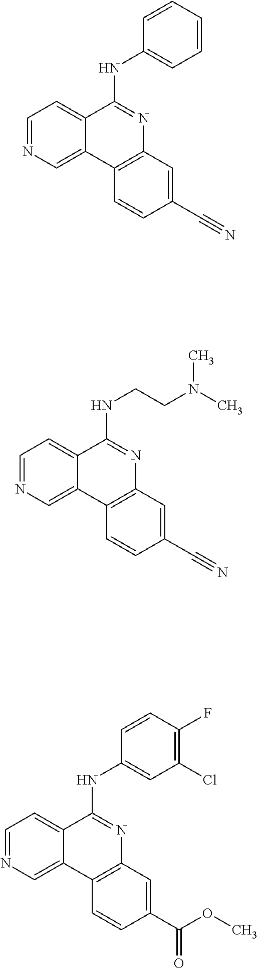

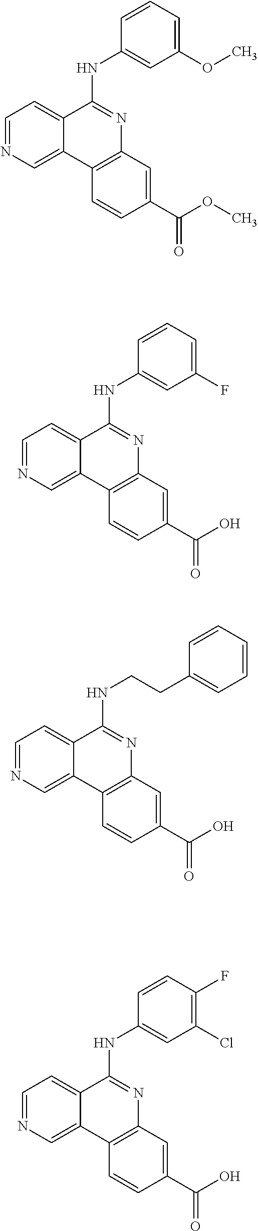

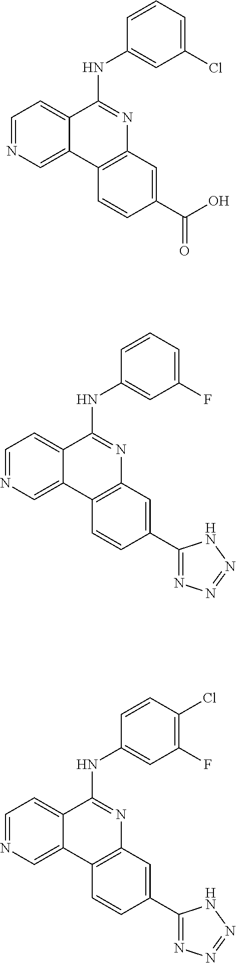

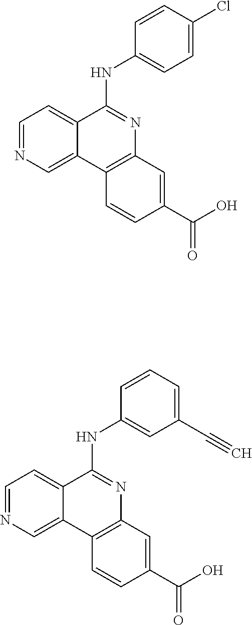

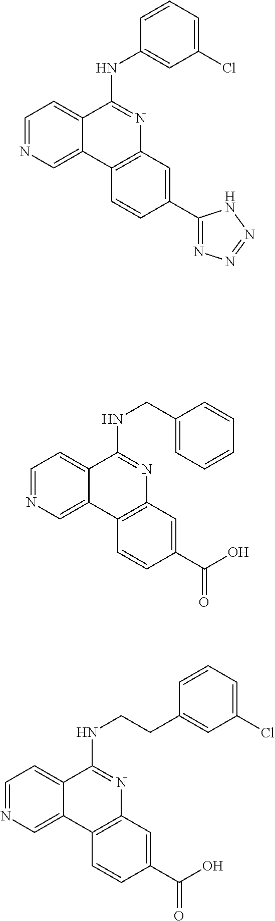

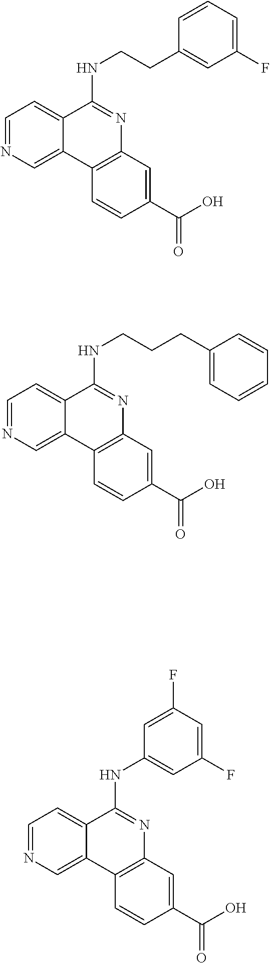

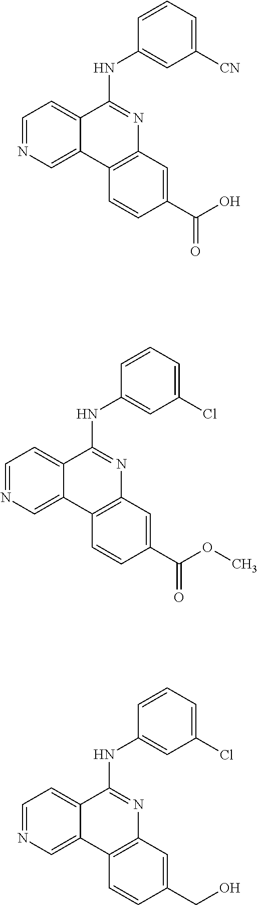

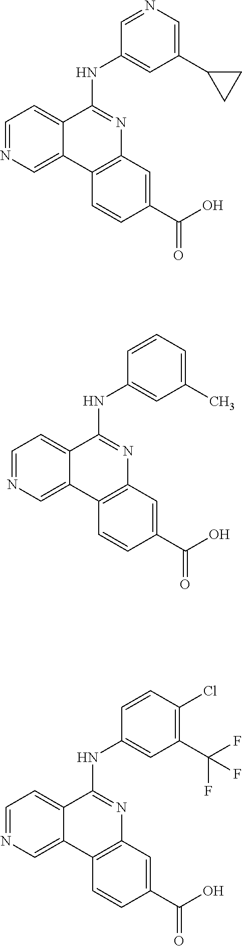

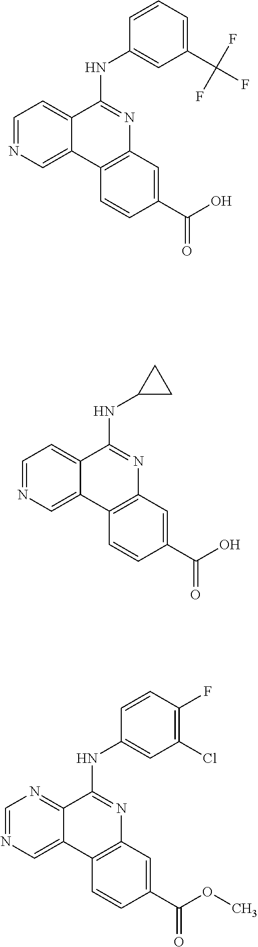

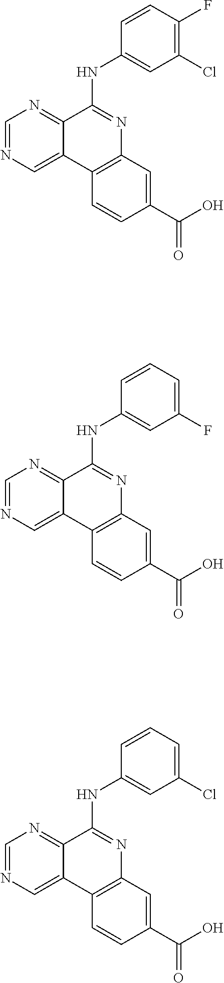

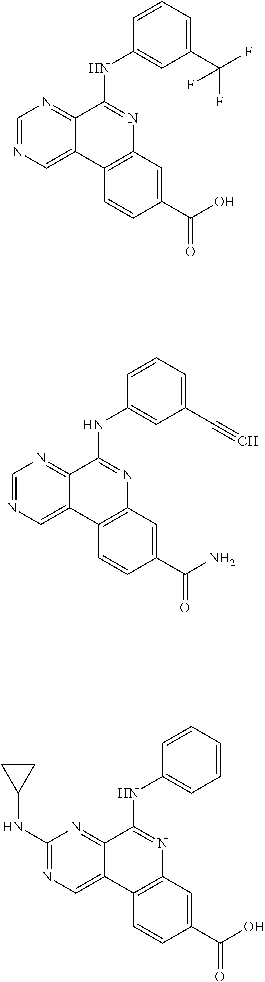

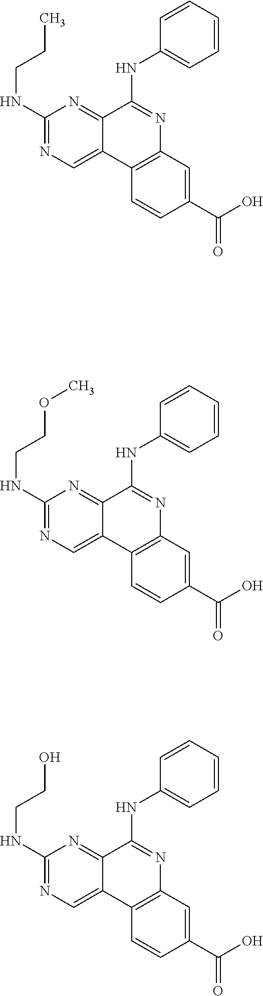

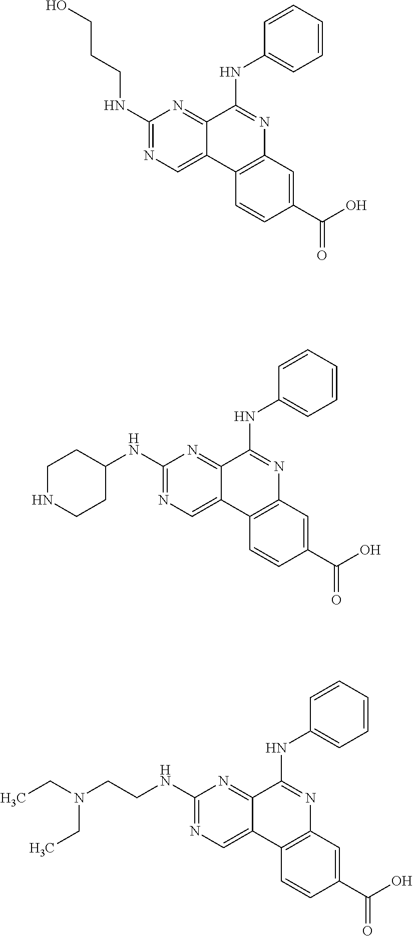

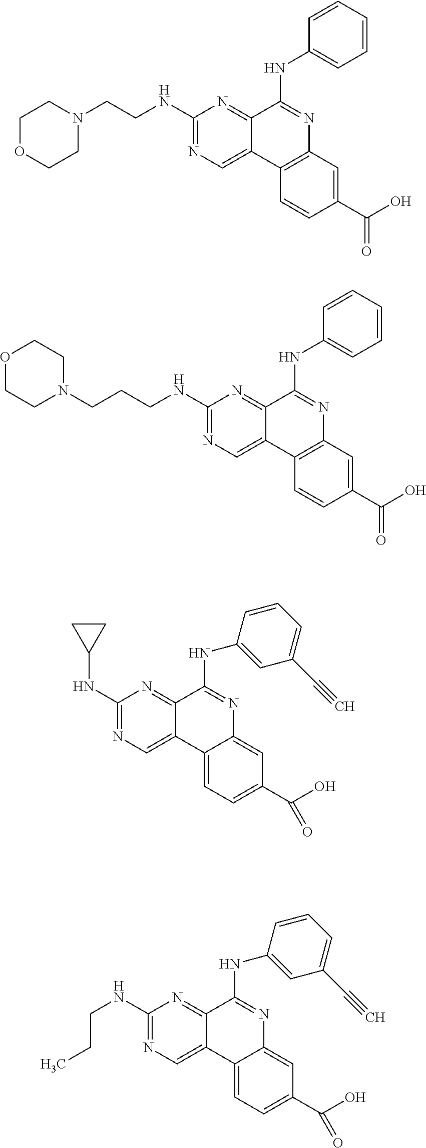

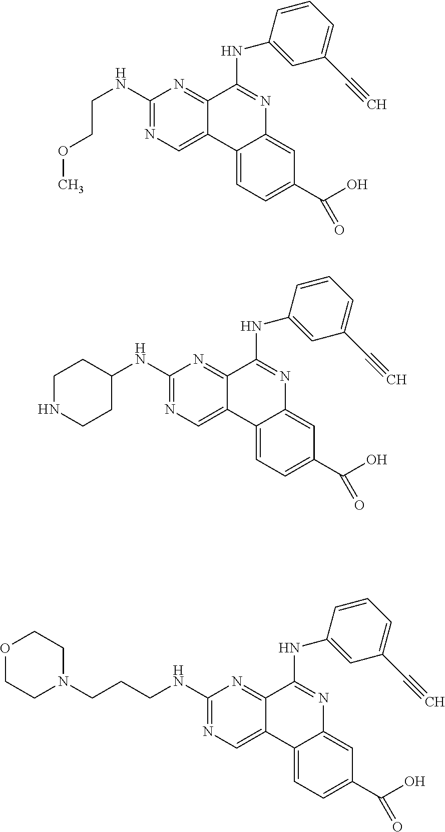

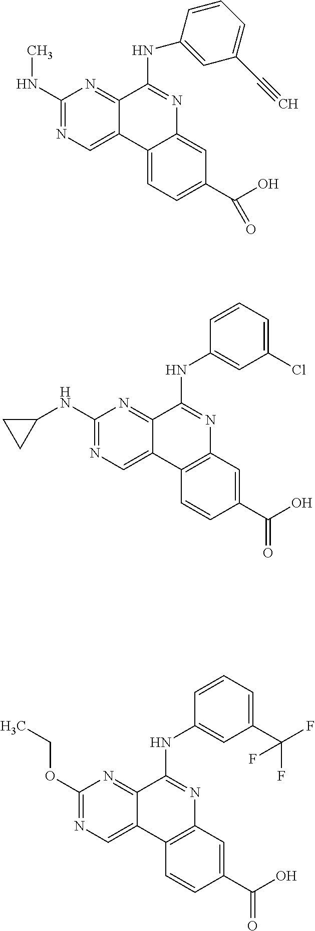

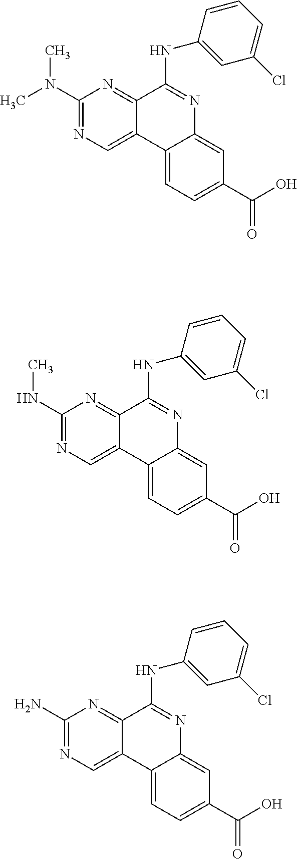

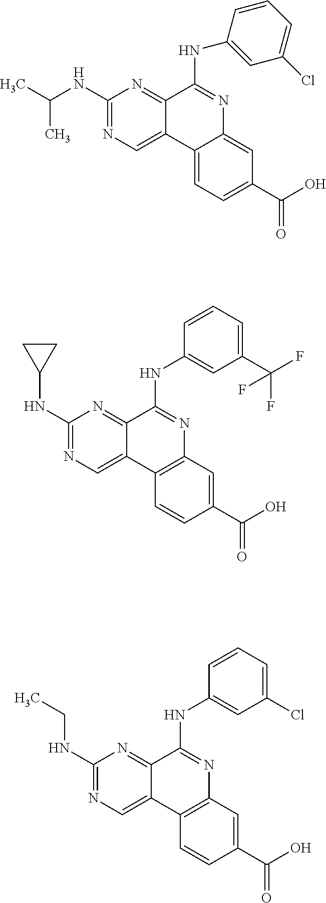

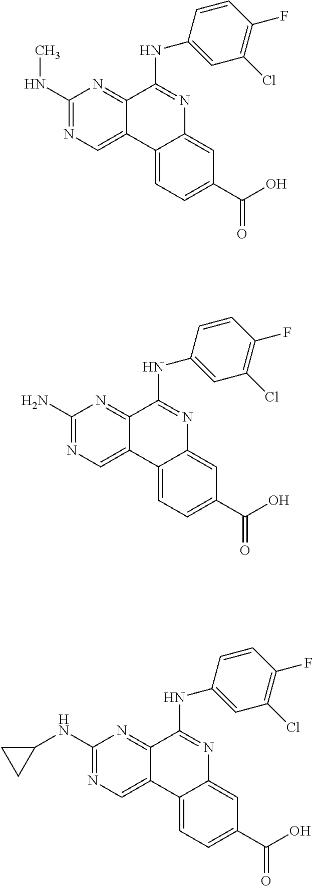

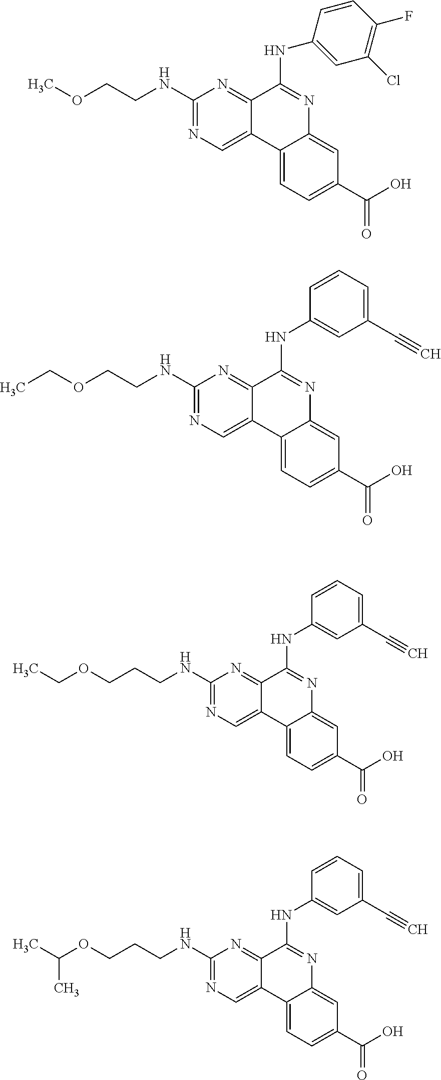

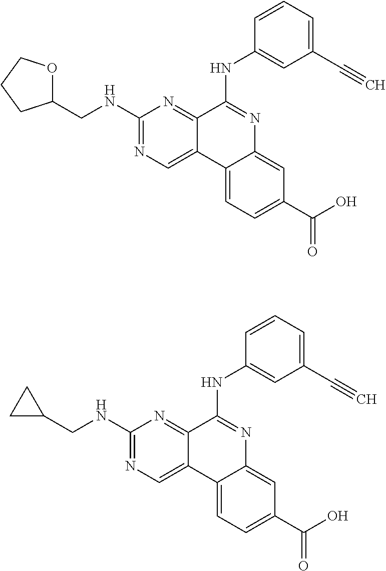

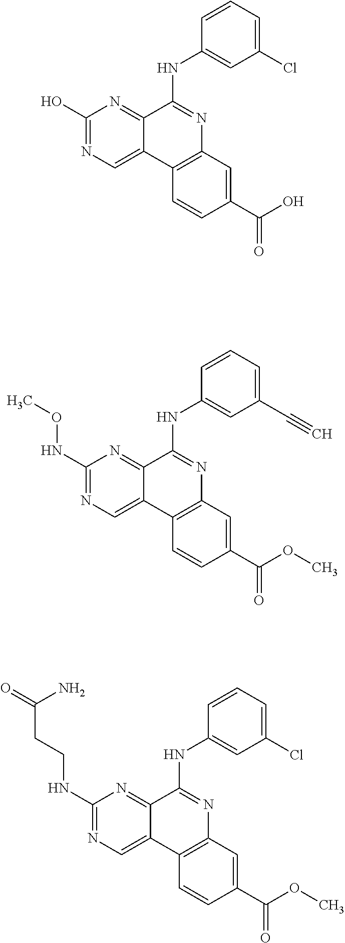

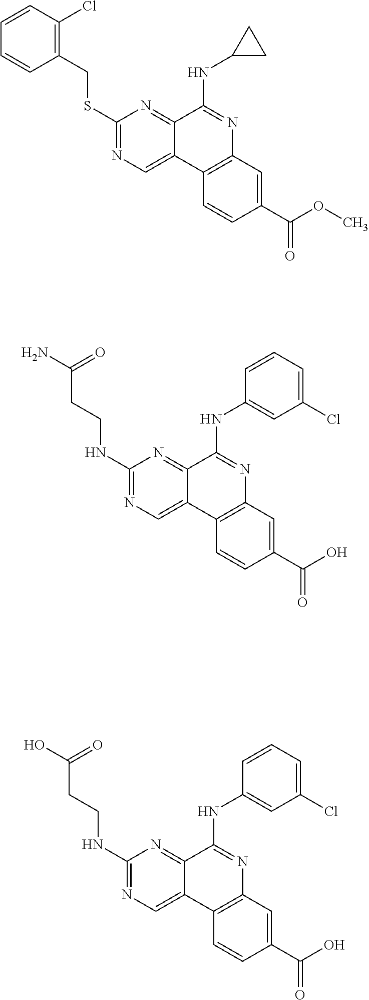

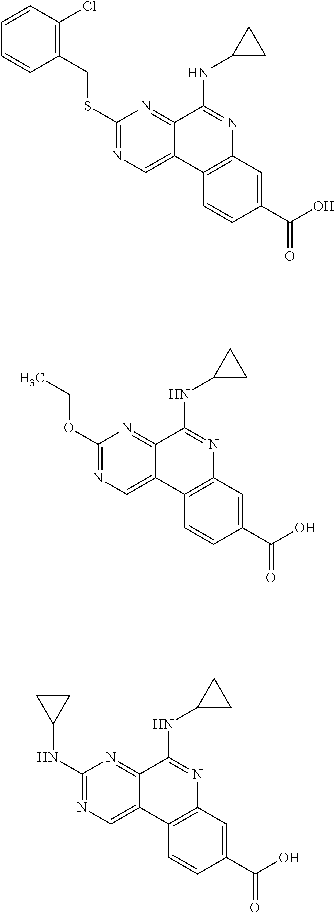

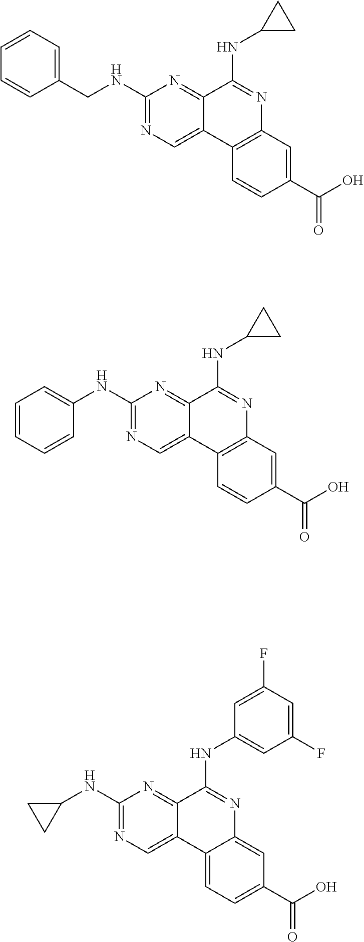

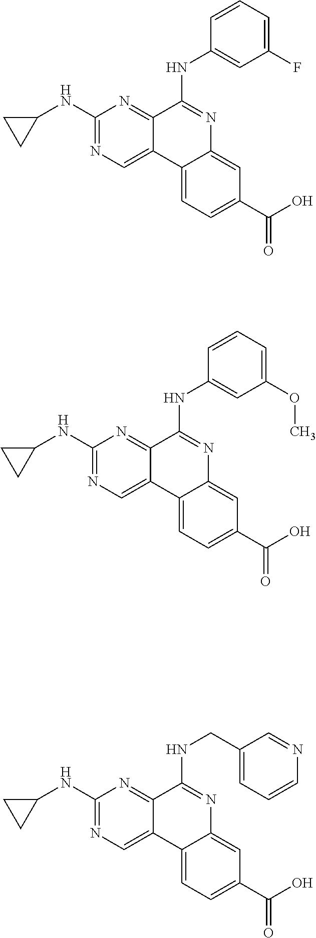

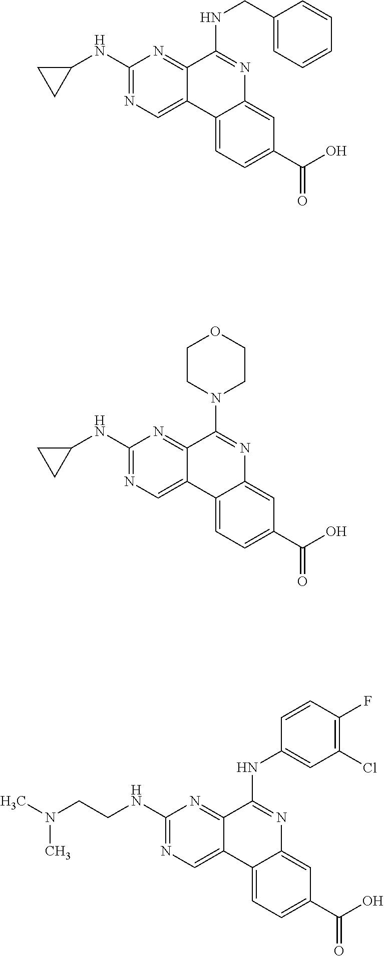

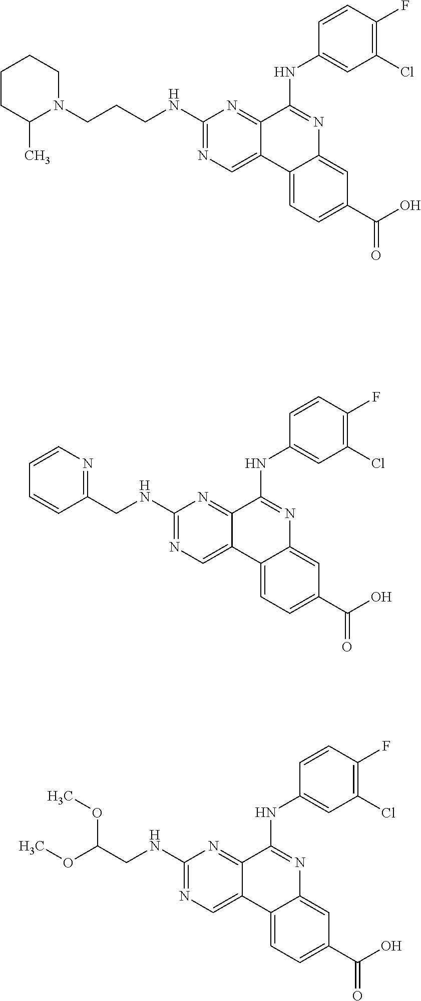

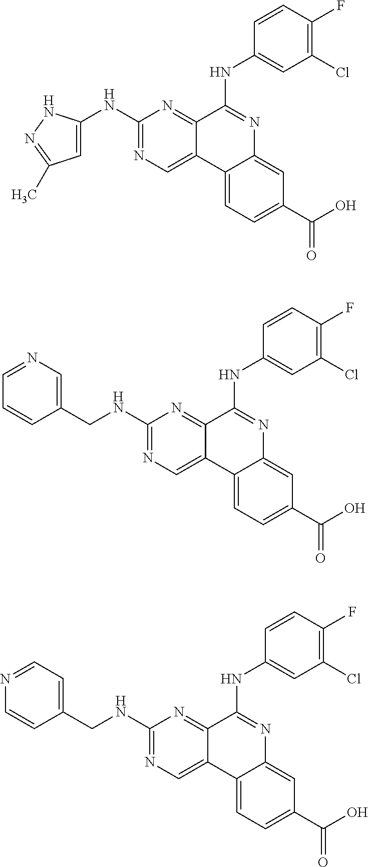

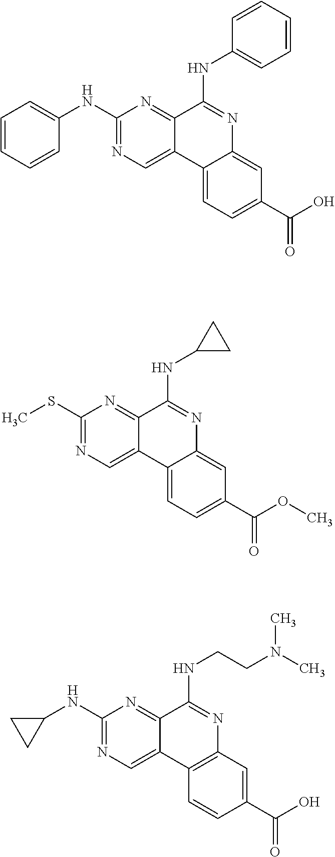

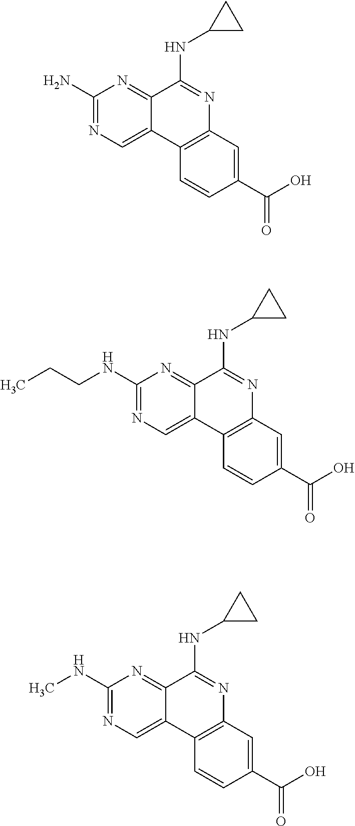

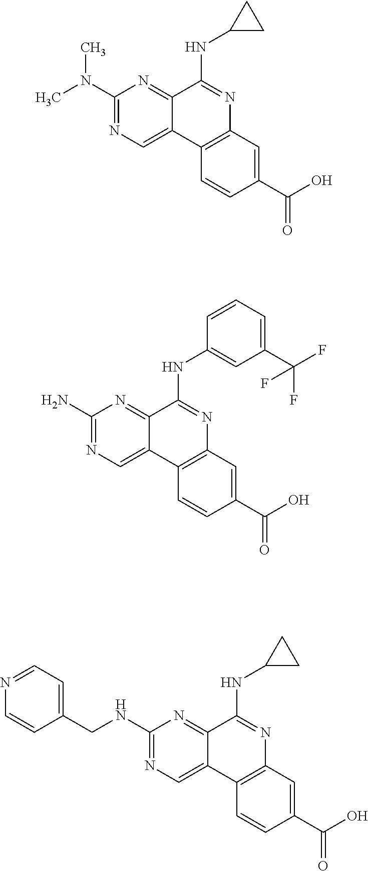

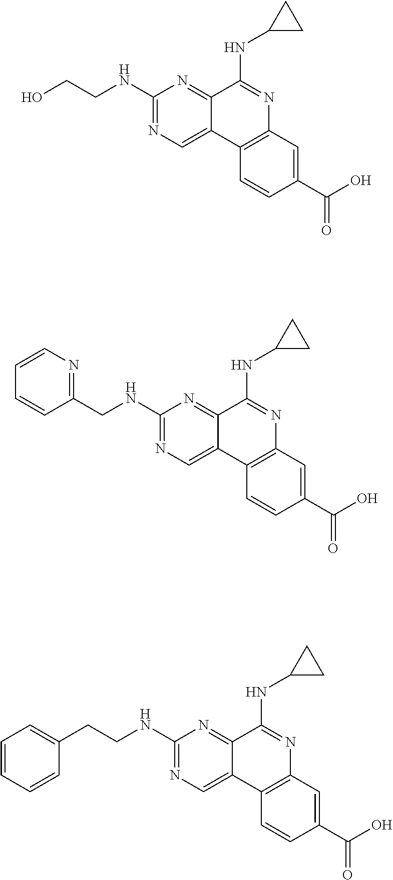

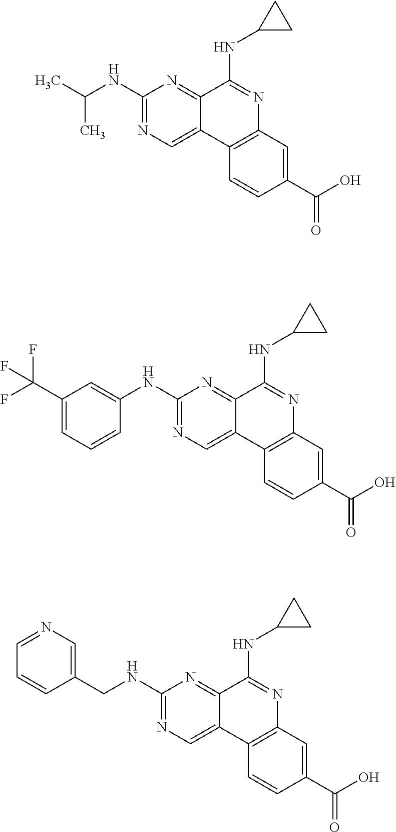

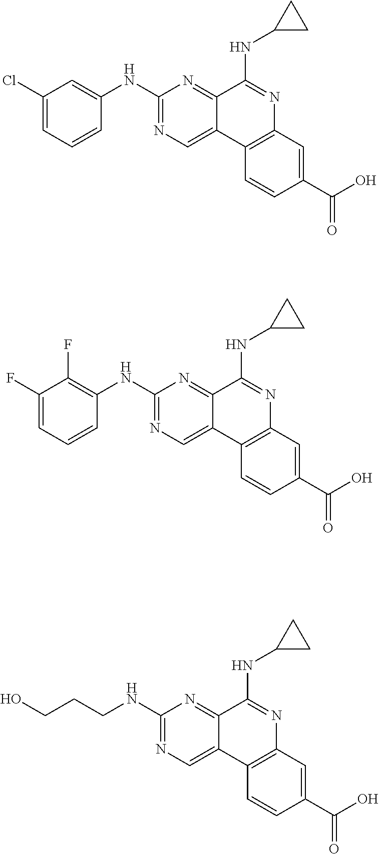

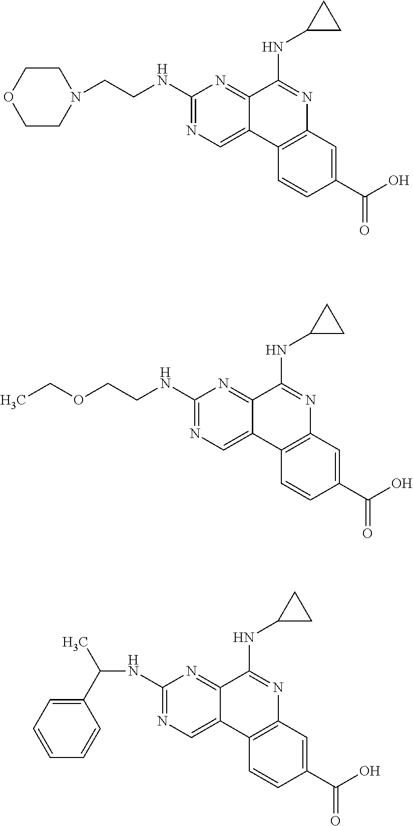

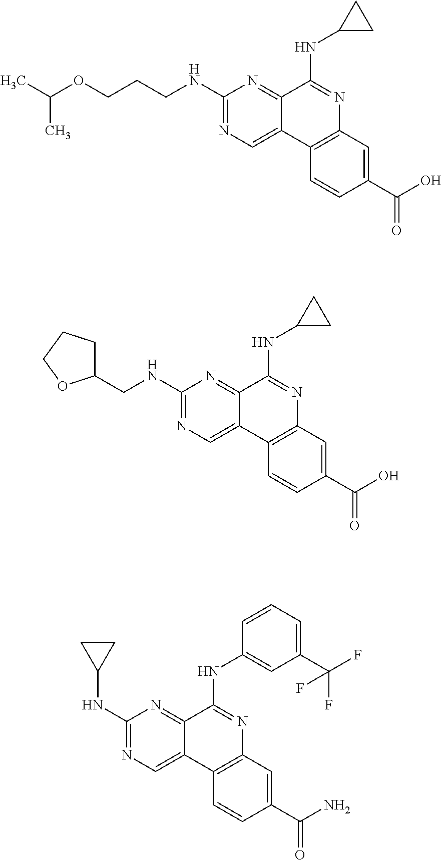

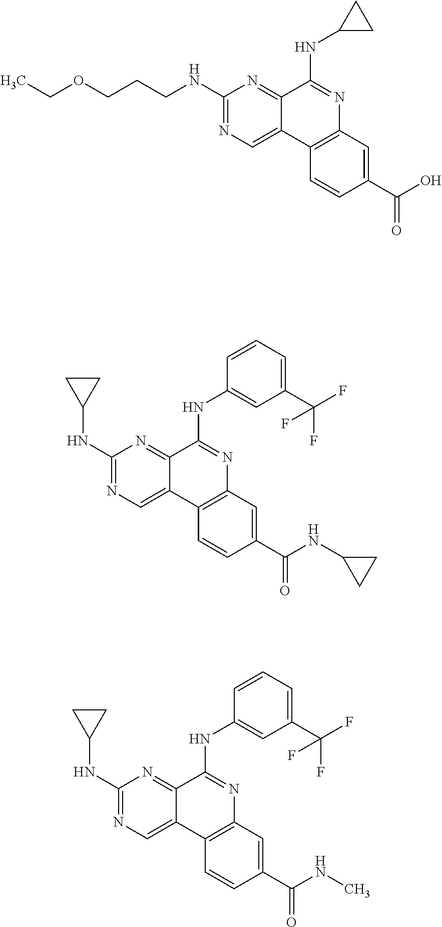

In another embodiment of the invention, the compound has a structural Formula (B1), (B2), or (B3):

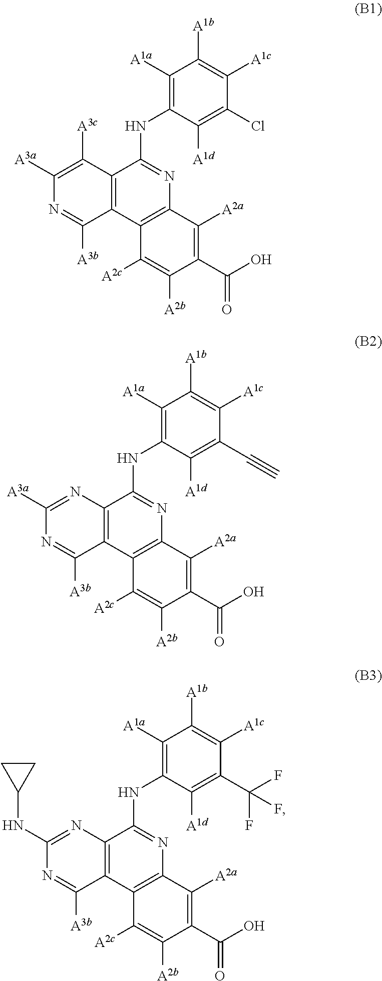

or or a pharmaceutically acceptable salt, solvate, and/or prodrug thereof;
   wherein:
each A^{1a}, A^{1b}, A^{c}, A^{1d}, A^{2a}, A^{2b}, A^{2c}, A^{3a}, A^{3b}, and A^{3c} is independently H or deuterium; and;
with the following provisos:
(a) at least one of A^{1a}, A^{1b}, A^{1c}, A^{1d}, A^{2a}, A^{2b}, A^{2c}, A^{3a}, A^{3b}, and A^{3c} in Formula (B1) is deuterium;
(b) at least one of A^{1a}, A^{1b}, A^{1c}, A^{1d}, A^{2a}, A^{2b}, A^{2c}, A^{3a}, and A^{3b} in Formula (B2) is deuterium; and
(c) at least one of A^{1a}, A^{1b}, A^{1c}, A^{1d}, A^{2a}, A^{2b}, A^{2c}, and A^{3b} in Formula (B3) is deuterium.

In yet another embodiment of the invention, the compound has a structural Formula (B1), (B2), or (B3) as described above, wherein
each A^{1a}, A^{1b}, A^{1c} and A^{1d}, is independently H or deuterium;
A^{2a}, A^{2b}, A^{2c}, A^{3a}, A^{3b}, and A^{3c} are H; and
with the proviso that at least one of A^{1a}, A^{1b}, A^{1c}, and A^{1d} is deuterium.

In yet another embodiment of the invention, the compound has a structural Formula (B1), (B2), or (B3) as described above, wherein
each A^{1a}, A^{1b}, A^{1c}, and A^{1d}, is independently H or deuterium;
each A^{2a}, A^{2b}, and A^{2c} is independently H or deuterium;
A^{3a}, A^{3b}, and A^{3c} are H; and
with the provisos that
(a) at least one of A^{1a}, A^{1b}, A^{1c}, and A^{1d} is deuterium; and
(b) at least one of A^{2a}, A^{2b}, and A^{2c} is deuterium.

In yet another embodiment of the invention, the compound has a structural Formula (B1), (B2), or (B3) as described above, wherein
each A^{2a}, A^{2b}, and A^{2c} is independently H or deuterium;
A^{1a}, A^{1b}, A^{1c}, A^{1d}, A^{3a}, A^{3b}, and A^{3c} are H; and
with the proviso that at least one of A^{2a}, A^{2b}, and A^{2c} is deuterium.

In yet another embodiment of the invention, the compound has a structural Formula (B1), (B2), or (B3) as described above, wherein
each A^{3a}, A^{3b}, and A^{3c} is independently H or deuterium;
A^{1a}, A^{1b}, A^{1c}, A^{1d}, A^{2a}, A^{2b}, and A^{2c} are H ; and
with the proviso that at least one of A^{3a}, A^{3b}, and A^{3c} is deuterium.

A further embodiment of the invention is a pharmaceutical composition comprising and compound as described above, or a pharmaceutically acceptable salt, solvate, and/or prodrug thereof; and a pharmaceutically acceptable carrier.

Another embodiment of the invention is a method of modulating a serine-threonine protein kinase activity in a cell, comprising contacting the cell with any one of the compounds as described above, or a pharmaceutically acceptable salt, solvate, and/or prodrug thereof in an amount effective to modulate a serine-threonine protein kinase activity.

Yet another embodiment of the invention is a method of inhibiting cell proliferation, comprising contacting cells with any one of the compounds as described above, or a pharmaceutically acceptable salt, solvate, and/or prodrug thereof in an amount effective to inhibit proliferation of the cells.

Another embodiment is either of the methods as described above, wherein the cells are in a cancer cell line or in a tumor in a subject.

A further embodiment of the invention is the method of treating a condition or disease related to aberrant cell proliferation, comprising administering a therapeutically effective amount of any one of the compounds as described above, or a pharmaceutically acceptable salt, solvate, and/or prodrug thereof, to a subject in need thereof.

Another embodiment of the invention is the method as described above, wherein the condition or disease is a tumor-associated cancer, a non-tumor cancer, or macular degeneration.

Another embodiment of the invention is the method as described above, wherein the non-tumor cancer is a hematopoietic cancer.

Yet another embodiment of the invention is a method of treating a condition or disease associated with a serine-threonine protein kinase activity, comprising administering a therapeutically effective amount of any one of the compounds as described above, or a pharmaceutically acceptable salt, solvate, and/or prodrug thereof, to a subject in need thereof.

In a further embodiment of the invention, the serine-threonine protein kinase is casein kinase 2.

Another embodiment of the invention is a method of treating a condition or disease associated with a serine-threonine protein kinase activity, wherein the condition or disease is selected from the group consisting of a cancer, an immunological disorder, a pathogenic infection, an inflammation, a pain, an angiogenesis-related disorder, and combination thereof.

Yet another embodiment of the invention is the method described above wherein the condition or disease is a cancer of colorectum, breast, lung, liver, pancreas, lymph node, colon, prostate, brain, head and neck, skin, liver, kidney, or blood and heart.

A further embodiment of the invention is a pharmaceutical composition comprising any one of the compounds as described above, or a pharmaceutically acceptable salt, solvate, and/or prodrug thereof; and at least one additional therapeutic agent.

Yet another embodiment of the invention is a method to treat a condition related to aberrant cell proliferation, which comprises co-administering to a subject in need of treatment for such condition any one of the compounds as described above, or a pharmaceutically acceptable salt, solvate, and/or prodrug thereof; and at least one additional therapeutic agent.

### Definitions:

It is to be understood that the terminology used herein is for the purpose of describing particular embodiments only and is not intended to be limiting. Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

The terms “a” and “an” do not denote a limitation of quantity, but rather denote the presence of at least one of the referenced item. The terms “a” and “an” are used interchangeable with “one or more” or “at least one”. The term “or” or “and/or” is used as a function word to indicate that two words or expressions are to be taken together or individually. The terms “comprising”, “having”, “including”, and “containing” are to be construed as open-ended terms (i.e., meaning “including, but not limited to”). The endpoints of all ranges directed to the same component or property are inclusive and independently combinable.

The terms “compound(s) of the invention”, “these compounds”, “such compound(s)”, “the compound(s)”, and “the present compound(s)” refer to compounds encompassed by structural formulae disclosed herein, e.g., Formula (A), (I), (II), (III), (IV), (Ia), (Ib), (Ic), (Id), (B 1), (B2), and (B3), including any specific compounds within these formulae whose structure is disclosed herein. Compounds may be identified either by their chemical structure and/or chemical name. When the chemical structure and chemical name conflict, the chemical structure is determinative of the identity of the compound. Furthermore, the present compounds can inhibit the biological activity of a CK2 protein, and thereby is also referred to herein as an “inhibitor(s)” or “CK2 inhibitor(s)”. Compound's of Formula (A), (I), (II), (III), (IV), (Ia), (Ib), (Ic), (Id), (B1), (B2), and (B3), including any specific compounds described herein are exemplary “inhibitors”. The descriptions of compounds of the invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The present compounds may contain one or more chiral centers and/or double bonds and therefore, may exist as stereoisomers, such as double-bond isomers (i.e., geometric isomers such as E and Z), enantiomers or diastereomers. The invention includes each of the isolated stereoisomeric forms as well as mixtures of stereoisomers in varying degrees of chiral purity, including racemic mixtures and mixtures of diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The invention includes each of the isolated stereoisomeric forms as well as mixtures of stereoisomers in varying degrees of chiral purity, including racemic mixtures. It also encompasses the various diastereomers. Other structures may appear to depict a specific isomer, but that is merely for convenience, and is not intended to limit the invention to the depicted olefin isomer.

The present compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein refers to isomers that change into one another with great ease so that they can exist together in equilibrium. For example, ketone and enol are two tautomeric forms of one compound. In another example, a substituted 1,2,4-triazole derivative may exist in at least three tautomeric forms as shown below:

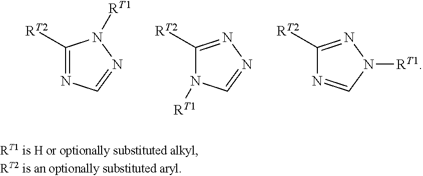

The term “deuterium” refers to an isotope of hydrogen that has one proton and one neutron in its nucleus and that has twice the mass of ordinary hydrogen. Deuterium can be represented by symbols such as “²H” or “D”. The term “deuterated” herein, by itself or used to modify a compound or group, refers to replacement of one or more hydrogen atom(s), which is attached to carbon(s), with a deuterium atom. For example, the term “deuterated compound” refers to a compound wherein one or more carbon-bound hydrogen(s) are replaced by one or more deuterium(s). Similarly, the term “deuterated” is be used herein to modify a chemical structure in phrases like “a deuterated form of the following structure” or “the following structure(s) in a deuterated form”; a chemical name, such as “deuterated-(2S,3S)-2-amino-3-methyl-N-(2-morpholinoethyl)-pentanamide”; or an organic group or radical, such as “deuterated-alkyl”, “deuterated-cycloalkyl”, “deuterated-heterocycloalkyl”, “deuterated-aryl”, “deuterated-morpholinyl”, and the like.

The phrase “deuterated-alkyl” refers to an alkyl group as defined herein, wherein at least one hydrogen atom bound to carbon is replaced by a deuterium. That is, in a deuterated alkyl group, at least one carbon atom is bound to a deuterium. In a deuterated alkyl group, it is possible for a carbon atom to be bound to more than one deuterium; it is also possible that more than one carbon atom in the alkyl group is bound to a deuterium. Analogously, the term “deuterated” and the phrases “deuterated-heterocycloalkyl,” deuterated-heteroaryl,” “deuterated-cycloalkyl,” “deuterated-heterocycloalkyl,” “deuterated-aryl,” “deuteratedy-acyl,” “deuterated-alkoxyl” each refer to the chemical moiety wherein one carbon chain is bound to a deuterium.

The phrase “corresponding undeuterated compound” or “protonated analog” refers to a compound having identical chemical structure as a deuterated compound except that all hydrogen are present at their natural isotopic abundance percentages.

It will be recognized that some variation of natural isotopic abundance occurs in a synthesized compound depending upon the origin of chemical materials used in the synthesis. Thus, a preparation of a compound will inherently contain small amounts of deuterated isotopologues. The concentration of naturally abundant stable hydrogen and carbon isotopes, notwithstanding this variation, is small and immaterial as compared to the degree of stable isotopic substitution of deuterated compounds of this disclosure. In a deuterated compound of this disclosure, when a particular position is designated as having deuterium, it is understood that the abundance of deuterium at that position is substantially greater than the natural abundance of deuterium, which is 0.015%. A position designated as having deuterium typically has a minimum isotopic enrichment factor of at least 3000 (45% deuterium incorporation) at each atom designated as deuterium in the compound.

The term “isotopic enrichment factor” as used herein means the ratio between the isotopic abundance and the natural abundance of a specified isotope.

In other embodiments, a compound of this disclosure has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition.

The term “isotopologue” refers to a species that has the same chemical structure and formula as a specific compound of this invention, with the exception of the isotopic composition at one or more positions, e.g., H vs. D. Thus an isotopologue differs from a specific compound of this invention in the isotopic composition thereof.

The compounds of the invention often have ionizable groups so as to be capable of preparation as salts. In that case, wherever reference is made to the compound, it is understood in the art that a pharmaceutically acceptable salt may also be used. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art. In some cases, the compounds may contain both an acidic and a basic functional group, in which case they may have two ionized groups and yet have no net charge. Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa.

“Solvate”, as used herein, means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate”. Examples of hydrate include, but are not limited to, hemihydrate, monohydrate, dihydrate, trihydrate, hexahydrate, etc. It should be understood by one of ordinary skill in the art that the pharmaceutically acceptable salt, and/or prodrug of the present compound may also exist in a solvate form. The solvate is typically formed via hydration which is either part of the preparation of the present compound or through natural absorption of moisture by the anhydrous compound of the present invention.

The term “ester” means any ester of a present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolysable esters of the present compounds are the compounds whose carboxyls are present in the form of hydrolysable ester groups. That is, these esters are pharmaceutically acceptable and can be hydrolyzed to the corresponding carboxyl acid in vivo. These esters may be conventional ones, including lower alkanoyloxyalkyl esters, e.g. pivaloyloxymethyl and 1-pivaloyloxyethyl esters; lower alkoxycarbonylalkyl esters, e.g., methoxycarbonyloxymethyl, 1-ethoxycarbonyloxyethyl, and 1-isopropylcarbonyloxyethyl esters; lower alkoxymethyl esters, e.g., methoxymethyl esters, lactonyl esters, benzofuran keto esters, thiobenzofuran keto esters; lower alkanoylaminomethyl esters, e.g., acetylaminomethyl esters. Other esters can also be used, such as benzyl esters and cyano methyl esters. Other examples of these esters include: (2,2-dimethyl-1-oxypropyloxy)methyl esters; (1RS)-1-acetoxyethyl esters, 2-[(2-methylpropyloxy)carbonyl]-2-pentenyl esters, 1-[[(1-methylethoxy)carbonyl]-oxy]ethyl esters; isopropyloxycarbonyloxyethyl esters, (5-methyl-2-oxo-1,3-dioxole-4-yl) methyl esters, 1-[[(cyclohexyloxy)carbonyl]oxy]ethyl esters; 3,3-dimethyl-2-oxobutyl esters. It is obvious to those skilled in the art that hydrolysable esters of the compounds of the present invention can be formed at free carboxyls of said compounds by using conventional methods. Representative esters include pivaloyloxymethyl esters, isopropyloxycarbonyloxyethyl esters and (5-methyl-2-oxo-1,3-dioxole-4-yl)methyl esters.

The term “solubility” as used herein, describes the maximum amount of solute, i.e., the present compound, that will dissolve in a given amount of solvent at a specified temperature.

The term “bioavailability” as used herein refers to the systemic availability (i.e., blood/plasma levels) of a given amount of a compound administered to a subject. The term further encompasses the rate and extent of absorption of a compound that reaches the site of action.

The term “prodrug” refers to a precursor of a pharmaceutically active compound wherein the precursor itself may or may not be pharmaceutically active but, upon administration, will be converted, either metabolically or otherwise, into the pharmaceutically active compound or drug of interest. For example, prodrug can be an ester, ether, or amide form of a pharmaceutically active compound. Various types of prodrug have been prepared and disclosed for a variety of pharmaceuticals. See, for example, Bundgaard, H. and Moss, J., J. Pharm. Sci. 78: 122-126 (1989). Thus, one of ordinary skill in the art knows how to prepare these prodrugs with commonly employed techniques of organic synthesis.

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

“Excipient” refers to a diluent, adjuvant, vehicle, or carrier with which a compound is administered.

An “effective amount” or “therapeutically effective amount” is the quantity of the present compound in which a beneficial outcome is achieved when the compound is administered to a patient or alternatively, the quantity of compound that possesses a desired activity in vivo or in vitro. In the case of proliferative disorders, a beneficial clinical outcome includes reduction in the extent or severity of the symptoms associated with the disease or disorder and/or an increase in the longevity and/or quality of life of the patient compared with the absence of the treatment. For example, for a subject with cancer, a “beneficial clinical outcome” includes a reduction in tumor mass, a reduction in the rate of tumor growth, a reduction in metastasis, a reduction in the severity of the symptoms associated with the cancer and/or an increase in the longevity of the subject compared with the absence of the treatment. The precise amount of compound administered to a subject will depend on the type and severity of the disease or condition and on the characteristics of the patient, such as general health, age, sex, body weight and tolerance to drugs. It will also depend on the degree, severity and type of proliferative disorder. The skilled artisan will be able to determine appropriate dosages depending on these and other factors.

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10 C or as C1-C10 or C1-10. When heteroatoms (N, O and S typically) are allowed to replace carbon atoms as in heteroalkyl groups, for example, the numbers describing the group, though still written as e.g. C1-C6, represent the sum of the number of carbon atoms in the group plus the number of such heteroatoms that are included as replacements for carbon atoms in the backbone of the ring or chain being described.

Typically, the alkyl, alkenyl and alkynyl substituents of the invention contain 1-10 C (alkyl) or 2-10 C (alkenyl or alkynyl). Preferably they contain 1-8 C (alkyl) or 2-8 C (alkenyl or alkynyl). Sometimes they contain 1-4 C (alkyl) or 2-4 C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often optionally substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCSNR₂, NRC(═NR)NR₂, NRCOOR, NRCOR, CN, C≡CR, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group. Where two R or R′ are present on the same atom (e.g., NR₂), or on adjacent atoms that are bonded together (e.g., —NR—C(O)R), the two R or R; groups can be taken together with the atoms they are connected to to form a 5-8 membered ring, which can be substituted with C1-C4 alkyl, C1-C4 acyl, halo, C1-C4 alkoxy, and the like, and can contain an additional heteroatom selected from N, O and S as a ring member.

“Optionally substituted” as used herein indicates that the particular group or groups being described may have no non-hydrogen substituents, or the group or groups may have one or more non-hydrogen substituents. If not otherwise specified, the total number of such substituents that may be present is equal to the number of H atoms present on the unsubstituted form of the group being described. Where an optional substituent is attached via a double bond, such as a carbonyl oxygen (═O), the group takes up two available valences, so the total number of substituents that may be included is reduced according to the number of available valences.

“Substituted,” when used to modify a specified group or radical, means that one or more hydrogen atoms of the specified group or radical are each, independently of one another, replaced with the same or different substituent(s).

Substituent groups useful for substituting saturated carbon atoms in the specified group or radical include, but are not limited to —R^{a}, halo, —O⁻, ═O, —OR^{b}, 'SR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O )O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR″, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}s may be taken together with the nitrogen atom to which they are bonded form a 4-, 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl. As another specific example, a substituted alkyl is meant to include -alkylene-O-alkyl, -alkylene-heteroaryl, -alkylene-cycloheteroalkyl, -alkylene-C(O)OR^{b}, -alkylene-C(O)NR^{b}R^{b}, and —CH₂—CH₂—C(O)—CH₃. The one or more substituent groups, taken together with the atoms to which they are bonded, may form a cyclic ring including cycloalkyl and cycloheteroalkyl.

Similarly, substituent groups useful for substituting unsaturated carbon atoms in the specified group or radical include, but are not limited to, —R^{a}, halo, —O⁻, —OR^{b}, —SR^{b}, —S⁻, —NR^{c}R^{c}, trihalomethyl, —CF₃, —CN, —OCN, —SCN, —NO, —NO₂, —N₃, —S(O)₂R^{b}, —S(O)₂O⁻, —S(O)₂O R^{b}, —OS(O)₂R^{b}, —OS(O)₂O⁻, —OS(O)₂OR^{b}, —P(O)(O)₂, —P(O)(OR^{b})(O⁻), —P(O)(OR^{b})(OR^{b}), —C(O)R^{b}, 'C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, _{—NR}^{b}C(NR^{b})R^{b} and —NR^{b}C(NR^{b})NR^{c}R^{c}, where R^{a}, R^{b} and R^{c} are as previously defined.

Substituent groups useful for substituting nitrogen atoms in heteroalkyl and cycloheteroalkyl groups include, but are not limited to, —R^{a}, —O⁻, —OR^{b}, —SR^{b}, —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O)₂, —P(O)(OR^{b})(O), —P(O )(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)O R^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

“Acetylene” substituents are 2-10 C alkynyl groups that are optionally substituted, and are of the formula —C≡C—R^{a}, wherein R^{a} is H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

and each R^{a} group is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂, wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O; and wherein two R′ can be linked to form a 3-7 membered ring optionally containing up to three heteroatoms selected from N, O and S. In some embodiments, R^{a} of —C≡C—R^{a} is H or Me. Where two R or R′ are present on the same atom (e.g., NR₂), or on adjacent atoms that are bonded together (e.g., —NR—C(O)R), the two R or R; groups can be taken together with the atoms they are connected to to form a 5-8 membered ring, which can be substituted with C1-C4 alkyl, C1-C4 acyl, halo, C1-C4 alkoxy, and the like, and can contain an additional heteroatom selected from N, O and S as a ring member.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical and preferred sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably the monocyclic heteroaryls contain 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCSNR₂, NRC(═NR)NR₂, NRCOOR, NRCOR, CN, C≡CR, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. Where two R or R′ are present on the same atom (e.g., NR₂), or on adjacent atoms that are bonded together (e.g., —NR—C(O)R), the two R or R; groups can be taken together with the atoms they are connected to to form a 5-8 membered ring, which can be substituted with C1-C4 alkyl, C1-C4 acyl, halo, C1-C4 alkoxy, and the like, and can contain an additional heteroatom selected from N, O and S as a ring member.

The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C1-C4 alkylene that is unsubstituted or is substituted with one or two C1-C4 alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. Thus —CH(Me)— and —C(Me)₂— may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Heteroform” as used herein refers to a derivative of a group such as an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Halo”, as used herein includes fluoro, chloro, bromo and iodo. Fluoro and chloro are often preferred.

“Amino” as used herein refers to NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle” refers to a cyclic compound containing only carbon atoms in the ring, whereas a “heterocycle” refers to a cyclic compound comprising a heteroatom. The carbocyclic and heterocyclic structures encompass compounds having monocyclic, bicyclic or multiple ring systems. As used herein, these terms also include rings that contain a double bond or two, as long as the ring is not aromatic.

As used herein, the term “heteroatom” refers to any atom that is not carbon or hydrogen, such as nitrogen, oxygen or sulfur.

Illustrative examples of heterocycles include but are not limited to tetrahydrofuran, 1,3-dioxolane, 2,3-dihydrofuran, pyran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4b]pyridine, piperazine, pyrazine, morpholine, thiomorpholine, imidazole, imidazolidine 2,4-dione, 1,3-dihydrobenzimidazol-2-one, indole, thiazole, benzothiazole, thiadiazole, thiophene, tetrahydro thiophene 1,1-dioxide, diazepine, triazole, guanidine, diazabicyclo[2.2.1]heptane, 2,5-diazabicyclo[2.2.1]heptane, 2,3,4,4a,9,9a-hexahydro-1H-β-carboline, oxirane, oxetane, tetrahydropyran, dioxane, lactones, aziridine, azetidine, piperidine, lactams, and may also encompass heteroaryls. Other illustrative examples of heteroaryls include but are not limited to furan, pyrrole, pyridine, pyrimidine, imidazole, benzimidazole and triazole.

As used herein, the term “inorganic substituent” refers to substituents that do not contain carbon or contain carbon bound to elements other than hydrogen (e.g., elemental carbon, carbon monoxide, carbon dioxide, and carbonate). Examples of inorganic substituents include but are not limited to nitro, halogen, azido, cyano, sulfonyls, sulfinyls, sulfonates, phosphates, etc.

The term “polar substituent” as used herein refers to any substituent having an electric dipole, and optionally a dipole moment (e.g., an asymmetrical polar substituent has a dipole moment and a symmetrical polar substituent does not have a dipole moment). Polar substituents include substituents that accept or donate a hydrogen bond, and groups that would carry at least a partial positive or negative charge in aqueous solution at physiological pH levels. In certain embodiments, a polar substituent is one that can accept or donate electrons in a non-covalent hydrogen bond with another chemical moiety.

In certain embodiments, a polar substituent is selected from a carboxy, a carboxy bioisostere or other acid-derived moiety that exists predominately as an anion at a pH of about 7 to 8 or higher. Other polar substituents include, but are not limited to, groups containing an OH or NH, an ether oxygen, an amine nitrogen, an oxidized sulfur or nitrogen, a carbonyl, a nitrile, and a nitrogen-containing or oxygen-containing heterocyclic ring whether aromatic or non-aromatic. In some embodiments, the polar substituent (represented by X) is a carboxylate or a carboxylate bioisostere.

“Carboxylate bioisostere” or “carboxy bioisostere” as used herein refers to a moiety that is expected to be negatively charged to a substantial degree at physiological pH. In certain embodiments, the carboxylate bioisostere is a moiety selected from the group consisting of:

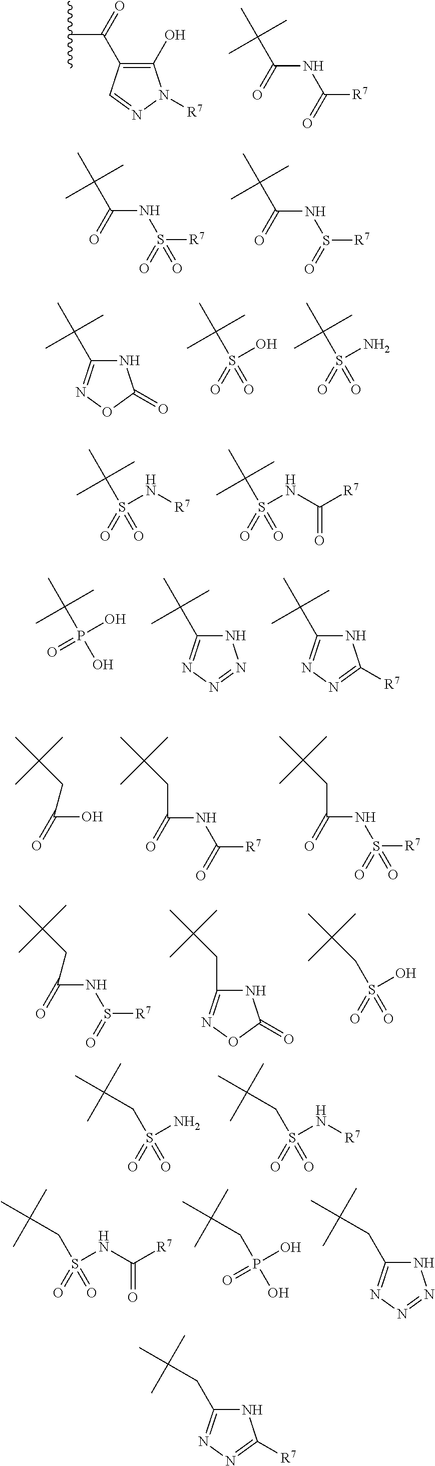

and salts of the foregoing, wherein each R⁷ is independently H or an optionally substituted member selected from the group consisting of C₁₋₁₀ alkyl, C₂₋₁₀ alkenyl, C₂₋₁₀ heteroalkyl, C₃₋₈ carbocyclic ring, and C₃₋₈ heterocyclic ring optionally fused to an additional optionally substituted carbocyclic or heterocyclic ring; or R⁷ is a C₁₋₁₀ alkyl, C₂₋₁₀ alkenyl, or C₂₋₁₀ heteroalkyl substituted with an optionally substituted C₃₋₈ carbocyclic ring or C₃₋₈ heterocyclic ring.

In certain embodiments, the polar substituent is selected from the group consisting of carboxylic acid, carboxylic ester, carboxamide, tetrazole, triazole, oxadiazole, oxothiadiazole, thiazole, aminothiazole, hydroxythiazole, and carboxymethanesulfonamide,. In some embodiments of the compounds described herein, at least one polar substituent present is a carboxylic acid or a salt, or ester or a bioisostere thereof. In certain embodiments, at least one polar substituent present is a carboxylic acid-containing substituent or a salt, ester or bioisostere thereof. In the latter embodiments, the polar substituent may be a C1-C10 alkyl or C1-C10 alkenyl linked to a carboxylic acid (or salt, ester or bioisostere thereof), for example.

The term ‘solgroup’ or ‘solubility-enhancing group’ as used herein refers to a molecular fragment selected for its ability to enhance physiological solubility of a compound that has otherwise relatively low solubility. Any substituent that can facilitate the dissolution of any particular molecule in water or any biological media can serve as a solubility-enhancing group. Examples of solubilizing groups are, but are not limited to: any substituent containing a group succeptible to being ionized in water at a pH range from 0 to 14; any ionizable group succeptible to form a salt; or any highly polar substituent, with a high dipolar moment and capable of forming strong interaction with molecules of water. Examples of solubilizing groups are, but are not limited to: substituted alkyl amines, substituted alkyl alcohols, alkyl ethers, aryl amines, pyridines, phenols, carboxylic acids, tetrazoles, sulfonamides, amides, sulfonylamides, sulfonic acids, sulfinic acids, phosphates, sulfonylureas.

Suitable groups for this purpose include, for example, groups of the formula -A-(CH₂)_{0.4}-G, where A is absent, O, or NR, where R is H or Me; and G can be a carboxy group, a carboxy bioisostere, hydroxy, phosphonate, sulfonate, or a group of the formula —NR^{y}₂ or P(O)(OR^{y})₂, where each R^{y} is independently H or a C1-C4 alkyl that can be substituted with one or more (typically up to three) of these groups: NH₂, OH, NHMe, NMe₂, OMe, halo, or ═O (carbonyl oxygen); and two Ry in one such group can be linked together to form a 5-7 membered ring, optionally containing an additional heteroatom (N, O or S) as a ring member, and optionally substituted with a C1-C4 alkyl, which can itself be substituted with one or more (typically up to three) of these groups: NH₂, OH, NHMe, NMe₂, OMe, halo, or ═O (carbonyl oxygen).

The terms “treat” and “treating” as used herein refer to ameliorating, alleviating, lessening, and removing symptoms of a disease or condition. A candidate molecule or compound described herein may be in a therapeutically effective amount in a formulation or medicament, which is an amount that can lead to a biological effect, such as apoptosis of certain cells (e.g., cancer cells), reduction of proliferation of certain cells, or lead to ameliorating, alleviating, lessening, or removing symptoms of a disease or condition, for example. The terms also can refer to reducing or stopping a cell proliferation rate (e.g., slowing or halting tumor growth) or reducing the number of proliferating cancer cells (e.g., removing part or all of a tumor). These terms also are applicable to reducing a titre of a microorganism in a system (i.e., cell, tissue, or subject) infected with a microorganism, reducing the rate of microbial propagation, reducing the number of symptoms or an effect of a symptom associated with the microbial infection, and/or removing detectable amounts of the microbe from the system. Examples of microorganisms include but are not limited to virus, bacterium and fungus.

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

### Utilities of the Compounds:

In another aspect, the invention provides a method to treat cancer, a vascular disorder, inflammation, or a pathogenic infection, comprising administering to a subject in need of such treatment, an effective amount of any of the above-described compounds.

In another aspect, the invention provides a method to inhibit cell proliferation, which comprises contacting cells with a compound of the invention, in an amount effective to inhibit proliferation of the cells. In certain embodiments, these cells are cells of a cancer cell line. In particular embodiments, the cancer cell line is a breast cancer, prostate cancer, pancreatic cancer, lung cancer, hemopoietic cancer, colorectal cancer, skin cancer, or an ovarian cancer cell line. Often, the cells are in a tumor in a subject, and the compound reduces the growth rate of the tumor, or reduces the size of the tumor, or reduces the aggressiveness of the tumor, or reduces the metastasis of the tumor. In some embodiments, the compound induces apoptosis.

In certain embodiments, the methods include contacting cells, especially tumor cells, with a compound of the invention, which induces apoptosis.

In certain embodiments, the cells are from an eye of a subject having macular degeneration, and the treatment method reduces the severity or symptoms or further development of macular degeneration in the subject.

In another aspect, the invention provides a method to treat a condition related to aberrant cell proliferation, which comprises administering a compound of the invention to a subject in need thereof, where the compound is administered in an amount effective to treat or ameliorate the cell proliferative condition. In certain embodiments, the cell proliferative condition is a tumor-associated cancer. Specific cancers for which the compounds are useful include breast cancer, prostate cancer, pancreatic cancer, lung cancer, hematopoietic cancer, colorectal cancer, skin cancer, and ovarian cancer, colorectum, liver, lymph node, colon, prostate, brain, head and neck, skin, kidney, blood and heart.

In other embodiments, the cell proliferative condition is a non-tumor cancer. Exemplary embodiments include hematopoietic cancers, such as lymphoma and leukemia.

In other embodiments, the cell proliferative condition is macular degeneration.

In another aspect, the invention provides a method for treating pain or inflammation in a subject, which comprises administering a compound of the invention to a subject in need thereof, in an amount effective to treat or reduce the pain or the inflammation.

In another aspect, the invention provides a method for inhibiting angiogenesis in a subject, which comprises administering a compound of the invention to a subject in need thereof in an amount effective to inhibit the angiogenesis.

The methods of treating these disorders comprise administering to a subject in need thereof an effective amount of an inhibitor compound of one of the formulae described herein.

The invention in part provides pharmaceutical compositions comprising at least one compound within the scope of the invention as described herein, and methods of using compounds described herein. For example, the invention in part provides methods for identifying a candidate molecule that interacts with a CK2 protein, which comprises contacting a composition containing a CK2 protein and a molecule described herein with a candidate molecule and determining whether the amount of the molecule described herein that interacts with the protein is modulated, whereby a candidate molecule that modulates the amount of the molecule described herein that interacts with the protein is identified as a candidate molecule that interacts with the protein.

Provided also are methods for modulating a protein kinase activity. Protein kinases catalyze the transfer of a gamma phosphate from adenosine triphosphate to a serine or threonine amino acid (serine/threonine protein kinase), tyrosine amino acid (tyrosine protein kinase), tyrosine, serine or threonine (dual specificity protein kinase) or histidine amino acid (histidine protein kinase) in a peptide or protein substrate. Thus, included herein are methods which comprise contacting a system comprising a protein kinase protein with a compound described herein in an amount effective for modulating (e.g., inhibiting) the activity of the protein kinase. In some embodiments, the activity of the protein kinase is the catalytic activity of the protein (e.g., catalyzing the transfer of a gamma phosphate from adenosine triphosphate to a peptide or protein substrate). In certain embodiments, provided are methods for identifying a candidate molecule that interacts with a protein kinase, which comprise: contacting a composition containing a protein kinase and a compound described herein with a candidate molecule under conditions in which the compound and the protein kinase interact, and determining whether the amount of the compound that interacts with the protein kinase is modulated relative to a control interaction between the compound and the protein kinase without the candidate molecule, whereby a candidate molecule that modulates the amount of the compound interacting with the protein kinase relative to the control interaction is identified as a candidate molecule that interacts with the protein kinase. Systems in such embodiments can be a cell-free system or a system comprising cells (e.g., in vitro). The protein kinase, the compound or the molecule in some embodiments is in association with a solid phase. In certain embodiments, the interaction between the compound and the protein kinase is detected via a detectable label, where in some embodiments the protein kinase comprises a detectable label and in certain embodiments the compound comprises a detectable label. The interaction between the compound and the protein kinase sometimes is detected without a detectable label.

Provided also are compositions of matter comprising a protein kinase and a compound described herein. In some embodiments, the protein kinase in the composition is a serine-threonine protein kinase or a tyrosine protein kinase. In certain embodiments, the protein kinase is a protein kinase fragment having compound-binding activity. In some embodiments, the protein kinase in the composition is, or contains a subunit (e.g., catalytic subunit, SH2 domain, SH3 domain) of CK2. In certain embodiments the composition is cell free and sometimes the protein kinase is a recombinant protein.

The protein kinase can be from any source, such as cells from a mammal, ape or human, for example. Examples of serine-threonine protein kinases that can be inhibited, or may potentially be inhibited, by compounds disclosed herein include without limitation human versions of CK2, CK2α2, Pim subfamily kinases (e.g., PIM1, PIM2, PIM3), CDK1/cyclinB, c-RAF, Mer, MELK, HIPK3, HIPK2 and ZIPK. A serine-threonine protein kinase sometimes is a member of a sub-family containing one or more of the following amino acids at positions corresponding to those listed in human CK2: leucine at position 45, methionine at position 163 and isoleucine at position 174. Examples of such protein kinases include without limitation human versions of CK2, STK10, HIPK2, HIPK3, DAPK3, DYK2 and PIM-1. Examples of tyrosine protein kinases that can be inhibited, or may potentially be inhibited, by compounds disclosed herein include without limitation human versions of Flt subfamily members (e.g., FLT1, FLT2, FLT3, FLT3 (D835Y), FLT4). An example of a dual specificity protein kinase that can be inhibited, or may potentially be inhibited, by compounds disclosed herein includes without limitation DYRK2. Nucleotide and amino acid sequences for protein kinases and reagents are publicly available (e.g., World Wide Web URLs ncbi.nlm.nih.gov/sites/entrez/ and Invitrogen.com). For example, various nucleotide sequences can be accessed using the following accession numbers: NM_{—}002648.2 and NP_{—}002639.1 for PIM1; NM_{—}006875.2 and NP_{—}006866.2 for PIM2; XM_{—}938171.2 and XP_{—}943264.2 for PIM3; NM_{—}004119.2 and NP_{—}004110.2 for FLT3; NM_{—}002020.3 and NP_{—}002011.2 for FLT4; and NM_{—}002019.3 and NP_{—}002010.2 for FLT1.

The invention also in part provides methods for treating a condition related to aberrant cell proliferation. For example, provided are methods of treating a cell proliferative condition in a subject, which comprises administering a compound described herein to a subject in need thereof in an amount effective to treat the cell proliferative condition. The subject may be a research animal (e.g., rodent, dog, cat, monkey), optionally containing a tumor such as a xenograft tumor (e.g., human tumor), for example, or may be a human. A cell proliferative condition sometimes is a tumor or non-tumor cancer, including but not limited to, cancers of the colorectum, breast, lung, liver, pancreas, lymph node, colon, prostate, brain, head and neck, skin, liver, kidney, blood and heart (e.g., leukemia, lymphoma, carcinoma).

Also provided are methods for treating a condition related to inflammation or pain. For example, provided are methods of treating pain in a subject, which comprise administering a compound described herein to a subject in need thereof in an amount effective to treat the pain. Provided also are methods of treating inflammation in a subject, which comprises administering a compound described herein to a subject in need thereof in an amount effective to treat the inflammation. The subject may be a research animal (e.g., rodent, dog, cat, monkey), for example, or may be a human. Conditions associated with inflammation and pain include without limitation acid reflux, heartburn, acne, allergies and sensitivities, Alzheimer's disease, asthma, atherosclerosis, bronchitis, carditis, celiac disease, chronic pain, Crohn's disease, cirrhosis, colitis, dementia, dermatitis, diabetes, dry eyes, edema, emphysema, eczema, fibromyalgia, gastroenteritis, gingivitis, heart disease, hepatitis, high blood pressure, insulin resistance, interstitial cystitis, joint pain/arthritis/rheumatoid arthritis, metabolic syndrome (syndrome X), myositis, nephritis, obesity, osteopenia, glomerulonephritis (GN), juvenile cystic kidney disease, and type I nephronophthisis (NPHP), osteoporosis, Parkinson's disease, Guam-Parkinson dementia, supranuclear palsy, Kuf's disease, and Pick's disease, as well as memory impairment, brain ischemia, and schizophrenia, periodontal disease, polyarteritis, polychondritis, psoriasis, scleroderma, sinusitis, Sjogren's syndrome, spastic colon, systemic candidiasis, tendonitis, urinary track infections, vaginitis, inflammatory cancer (e.g., inflammatory breast cancer) and the like. Methods for determining effects of compounds herein on pain or inflammation are known. For example, formalin-stimulated pain behaviors in research animals can be monitored after administration of a compound described herein to assess treatment of pain (e.g., Li et al., Pain 115(1-2): 182-90 (2005)). Also, modulation of pro-inflammatory molecules (e.g., IL-8, GRO-alpha, MCP-1, TNFalpha and iNOS) can be monitored after administration of a compound described herein to assess treatment of inflammation (e.g., Parhar et al., Int J Colorectal Dis. 22(6): 601-9 (2006)), for example. Thus, also provided are methods for determining whether a compound herein reduces inflammation or pain, which comprise contacting a system with a compound described herein in an amount effective for modulating (e.g., inhibiting) the activity of a pain signal or inflammation signal. Provided also are methods for identifying a compound that reduces inflammation or pain, which comprise: contacting a system with a compound of one of the formulae described herein; and detecting a pain signal or inflammation signal, whereby a compound that modulates the pain signal relative to a control molecule is identified as a compound that reduces inflammation of pain. Non-limiting examples of pain signals are formalin-stimulated pain behaviors and examples of inflammation signals include without limitation a level of a pro-inflammatory molecule. The invention thus in part pertains to methods for modulating angiogenesis in a subject, and methods for treating a condition associated with aberrant angiogenesis in a subject. proliferative diabetic retinopathy.

CK2 has also been shown to play a role in the pathogenesis of atherosclerosis, and may prevent atherogenesis by maintaining laminar shear stress flow. CK2 plays a role in vascularization, and has been shown to mediate the hypoxia-induced activation of histone deacetylases (HDACs). CK2 is also involved in diseases relating to skeletal muscle and bone tissue, including, e.g., cardiomyocyte hypertrophy, heart failure, impaired insulin signaling and insulin resistance, hypophosphatemia and inadequate bone matrix mineralization.

Thus in one aspect, the invention provides methods to treat these conditions, comprising administering to a subject in need of such treatment an effect amount of a CK2 inhibitor, such as a compound of one of the formulae disclosed herein.

Also provided are methods for treating an angiogenesis condition, which comprise administering a compound described herein to a subject in need thereof, in an amount effective to treat the angiogenesis condition. Angiogenesis conditions include without limitation solid tumor cancers, varicose disease, and the like.

Also provided are methods for treating a condition associated with an aberrant immune response in a subject, which comprise administering a compound described herein to a subject in need thereof, in an amount effective to treat the condition. Conditions characterized by an aberrant immune response include without limitation, organ transplant rejection, asthma, autoimmune disorders, including rheumatoid arthritis, multiple sclerosis, myasthenia gravis, systemic lupus erythematosus, scleroderma, polymyositis, mixed connective tissue disease (MCTD),Crohn's disease, and ulcerative colitis. In certain embodiments, an immune response may be modulated by administering a compound herein in combination with a molecule that modulates (e.g., inhibits) the biological activity of an mTOR pathway member or member of a related pathway (e.g., mTOR, PI3 kinase, AKT). In certain embodiments the molecule that modulates the biological activity of an mTOR pathway member or member of a related pathway is rapamycin. In certain embodiments, provided herein is a composition comprising a compound described herein in combination with a molecule that modulates the biological activity of an mTOR pathway member or member of a related pathway, such as rapamycin, for example.

In certain embodiments of the present invention, the compound is a compound of the invention, or a pharmaceutically acceptable salt, solvate, and/or prodrug of one of these compounds.

### Compositions and Modes of Administration:

In another aspect, the invention provides pharmaceutical compositions (i.e., formulations). The pharmaceutical compositions can comprise a compound of the present invention, as described herein which is admixed with at least one pharmaceutically acceptable excipient or carrier. Frequently, the composition comprises at least two pharmaceutically acceptable excipients or carriers.

While the compositions and methods of the present invention will typically be used in therapy for human patients, they may also be used in veterinary medicine to treat similar or identical diseases. The compositions may, for example, be used to treat mammals, including, but not limited to, primates and domesticated mammals. The compositions may, for example be used to treat herbivores. The compositions of the present invention include geometric and optical isomers of one or more of the drugs, wherein each drug is a racemic mixture of isomers or one or more purified isomers.

Pharmaceutical compositions suitable for use in the present invention include compositions wherein the active ingredients are contained in an effective amount to achieve the intended purpose. Determination of the effective amounts is well within the capability of those skilled in the art, especially in light of the detailed disclosure provided herein.

The compounds of the invention may exist as pharmaceutically acceptable salts. The present invention includes such salts. The term “pharmaceutically acceptable salts” is meant to include salts of active compounds which are prepared with relatively nontoxic acids or bases, depending on the particular substituent moieties found on the compounds described herein. When compounds of the present invention contain relatively acidic functionalities, base addition salts can be obtained by contacting the neutral form of such compounds with a sufficient amount of the desired base, either neat or in a suitable inert solvent. Included are base addition salts such as sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids, for example, acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Examples of applicable salt forms include hydrochlorides, hydrobromides, sulfates, methanesulfonates, nitrates, maleates, acetates, citrates, fumarates, tartrates (eg(+)-tartrates, (−)-tartrates or mixtures thereof, including racemic mixtures), succinates, benzoates and salts with amino acids such as glutamic acid. These salts may be prepared by methods known to those skilled in art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

The pharmaceutically acceptable esters in the present invention refer to non-toxic esters, preferably the alkyl esters such as methyl, ethyl, propyl, isopropyl, butyl, isobutyl or pentyl esters, of which the methyl ester is preferred. However, other esters such as phenyl-C₁₋₅ alkyl may be employed if desired. Ester derivatives of certain compounds may act as prodrugs which, when absorbed into the bloodstream of a warm-blooded animal, may cleave in such a manner as to release the drug form and permit the drug to afford improved therapeutic efficacy.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

When used as a therapeutic the compounds described herein often are administered with a physiologically acceptable carrier. A physiologically acceptable carrier is a formulation to which the compound can be added to dissolve it or otherwise facilitate its administration. Examples of physiologically acceptable carriers include, but are not limited to, water, saline, physiologically buffered saline.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention. The compounds of the present invention may also contain unnatural proportions of atomic isotopes at one or more of atoms that constitute such compounds. For example, the compounds may be radiolabeled with radioactive isotopes, such as for example tritium (3H), iodine-125 (¹²⁵I) or carbon-14 (¹⁴C). All isotopic variations of the compounds of the present invention, whether radioactive or not, are encompassed within the scope of the present invention.

Any suitable formulation of a compound described above can be prepared for administration. Any suitable route of administration may be used, including, but not limited to, oral, parenteral, intravenous, intramuscular, transdermal, topical and subcutaneous routes. Depending on the subject to be treated, the mode of administration, and the type of treatment desired—e.g., prevention, prophylaxis, therapy; the compounds are formulated in ways consonant with these parameters. Preparation of suitable formulations for each route of administration are known in the art. A summary of such formulation methods and techniques is found in Remington's Pharmaceutical Sciences, latest edition, Mack Publishing Co., Easton, Pa., which is incorporated herein by reference. Other examples of drug formulations can be found in Liberman, H. A. and Lachman, L., Eds., Pharmaceutical Dosage Forms, Marcel Decker, New York, N.Y., 1980. The formulation of each substance or of the combination of two substances will generally include a diluent as well as, in some cases, adjuvants, buffers, preservatives and the like. The substances to be administered can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, p1-1 buffering agents and the like, such as, for example, sodium acetate, sorbitan monolaurate, and so forth.

Various sustained release systems for drugs have also been devised, and can be applied to compounds of the invention. See, for example, U.S. Pat. No. 5,624,677, the methods of which are incorporated herein by reference.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

For administration to animal or human subjects, the appropriate dosage of the a compound described above often is 0.01 to 15 mg/kg, and sometimes 0.1 to 10 mg/kg. Dosage levels are dependent on the nature of the condition, drug efficacy, the condition of the patient, the judgment of the practitioner, and the frequency and mode of administration; however, optimization of such parameters is within the ordinary level of skill in the art.

### Therapeutic Combinations:

Compounds of the invention may be used alone or in combination with another therapeutic agent. The invention provides methods to treat conditions such as cancer, inflammation and immune disorders by administering to a subject in need of such treatment a therapeutically effective amount of a therapeutic agent useful for treating said disorder and administering to the same subject a therapeutically effective amount of a modulator of the present invention, i.e., a compound of the invention. The therapeutic agent and the modulator may be “co-administered”, i.e, administered together, either as separate pharmaceutical compositions or admixed in a single pharmaceutical composition. By “administered together”, the therapeutic agent and the modulator may also be administered separately, including at different times and with different frequencies. The modulator may be administered by any known route, such as orally, intravenously, intramuscularly, nasally, and the like; and the therapeutic agent may also be administered by any conventional route. In many embodiments, at least one and optionally both of the modulator and the therapeutic agent may be administered orally. Preferably, the modulator is an inhibitor, and it may inhibit either one of CK2 and Pim, or both of them to provide the treatment effects described herein.

In certain embodiments, a “modulator” as described above may be used in combination with a therapeutic agent that can act by binding to regions of DNA that can form certain quadruplex structures. In such embodiments, the therapeutic agents have anticancer activity on their own, but their activity is enhanced when they are used in combination with a modulator. This synergistic effect allows the therapeutic agent to be administered in a lower dosage while achieving equivalent or higher levels of at least one desired effect.

A modulator may be separately active for treating a cancer. For combination therapies described above, when used in combination with a therapeutic agent, the dosage of a modulator will frequently be two-fold to ten-fold lower than the dosage required when the modulator is used alone to treat the same condition or subject. Determination of a suitable amount of the modulator for use in combination with a therapeutic agent is readily determined by methods known in the art.

Compounds and compositions of the invention may be used in combination with anticancer or other agents, such as palliative agents, that are typically administered to a patient being treated for cancer. Such “anticancer agents” include, e.g., classic chemotherapeutic agents, as well as molecular targeted therapeutic agents, biologic therapy agents, and radiotherapeutic agents.

When a compound or composition of the invention is used in combination with an anticancer agent to another agent, the present invention provides, for example, simultaneous, staggered, or alternating treatment. Thus, the compound of the invention may be administered at the same time as an anticancer agent, in the same pharmaceutical composition; the compound of the invention may be administered at the same time as the anticancer agent, in separate pharmaceutical compositions; the compound of the invention may be administered before the anticancer agent, or the anticancer agent may be administered before the compound of the invention, for example, with a time difference of seconds, minutes, hours, days, or weeks.

In examples of a staggered treatment, a course of therapy with the compound of the invention may be administered, followed by a course of therapy with the anticancer agent, or the reverse order of treatment may be used, and more than one series of treatments with each component may also be used. In certain examples of the present invention, one component, for example, the compound of the invention or the anticancer agent, is administered to a mammal while the other component, or its derivative products, remains in the bloodstream of the mammal. For example, the compound of the invention may be administered while the anticancer agent or its derivative products remains in the bloodstream, or the anticancer agent may be administered while the compound of the invention or its derivatives remains in the bloodstream. In other examples, the second component is administered after all, or most of the first component, or its derivatives, have left the bloodstream of the mammal.

The compound of the invention and the anticancer agent may be administered in the same dosage form, e.g., both administered as intravenous solutions, or they may be administered in different dosage forms, e.g., one compound may be administered topically and the other orally. A person of ordinary skill in the art would be able to discern which combinations of agents would be useful based on the particular characteristics of the drugs and the cancer involved.

Anticancer agents useful in combination with the compounds of the present invention may include agents selected from any of the classes known to those of ordinary skill in the art, including, but not limited to, antimicrotubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; nonreceptor tyrosine kinase angiogenesis inhibitors; immunotherapeutic agents; pro-apoptotic agents; and cell cycle signaling inhibitors; and other agents described below.

Anti-microtubule or anti-mitotic agents are phase specific agents that are typically active against the microtubules of tumor cells during M or the mitosis phase of the cell cycle. Examples of anti-microtubule agents include, but are not limited to, diterpenoids and vinca alkaloids.

Plant alkaloid and terpenoid derived agents include mitotic inhibitors such as the vinca alkaloids vinblastine, vincristine, vindesine, and vinorelbine; and microtubule polymer stabilizers such as the taxanes, including, but not limited to paclitaxel, docetaxel, larotaxel, ortataxel, and tesetaxel.

Diterpenoids, which are derived from natural sources, are phase specific anti-cancer agents that are believed to operate at the G2/M phases of the cell cycle. It is believed that the diterpenoids stabilize the p-tubulin subunit of the microtubules, by binding with this protein. Disassembly of the protein appears then to be inhibited with mitosis being arrested and cell death following.

Examples of diterpenoids include, but are not limited to, taxanes such as paclitaxel, docetaxel, larotaxel, ortataxel, and tesetaxel. Paclitaxel is a natural diterpene product isolated from the Pacific yew tree Taxus brevifolia and is commercially available as an injectable solution TAXOL®. Docetaxel is a semisynthetic derivative of paclitaxel q. v., prepared using a natural precursor, 10-deacetyl-baccatin III, extracted from the needle of the European Yew tree. Docetaxel is commercially available as an injectable solution as TAXOTERE®.

Vinca alkaloids are phase specific anti-neoplastic agents derived from the periwinkle plant. Vinca alkaloids that are believed to act at'the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, vindesine, and vinorelbine. Vinblastine, vincaleukoblastine sulfate, is commercially available as VELBAN® as an injectable solution. Vincristine, vincaleukoblastine 22-oxo-sulfate, is commercially available as ONCOVIN® as an injectable solution. Vinorelbine, is commercially available as an injectable solution of vinorelbine tartrate (NAVELBINE®), and is a semisynthetic vinca alkaloid derivative.

Platinum coordination complexes are non-phase specific anti-cancer agents, which are interactive with DNA. The platinum complexes are believed to enter tumor cells, undergo, aquation and form intra- and interstrand crosslinks with DNA causing adverse biological effects to the tumor. Platinum-based coordination complexes include, but are not limited to cisplatin, carboplatin, nedaplatin, oxaliplatin, satraplatin, and (SP-4-3)-(cis)-amminedichloro-[2-methylpyridine]platinum(II). Cisplatin, cis-diamminedichloroplatinum, is commercially available as PLATINOL® as an injectable solution. Carboplatin, platinum, diammine [1,1-cyclobutane-dicarboxylate(2-)-0,0′], is commercially available as PARAPLATIN® as an injectable solution.

Alkylating agents are generally non-phase specific agents and typically are strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, alkyl sulfonates such as busulfan; ethyleneimine and methylmelamine derivatives such as altretamine and thiotepa; nitrogen mustards such as chlorambucil, cyclophosphamide, estramustine, ifosfamide, mechlorethamine, melphalan, and uramustine; nitrosoureas such as carmustine, lomustine, and streptozocin; triazenes and imidazotetrazines such as dacarbazine, procarbazine, temozolamide, and temozolomide. Cyclophosphamide, 2-[bis(2-chloroethyl)-amino]tetrahydro-2H-1,3,2-oxazaphosphorine 2-oxide monohydrate, is commercially available as an injectable solution or tablets as CYTOXAN®. Melphalan, 4-[bis(2-chloroethyl)amino]-L-phenylalanine, is commercially available as an injectable solution or tablets as ALKERAN®. Chlorambucil, 4-[bis(2-chloroethypamino]-benzenebutanoic acid, is commercially available as LEUKERAN® tablets. Busulfan, 1,4-butanediol dimethanesulfonate, is commercially available as MYLERAN® TABLETS. Carmustine, 1,3-[bis(2-chloroethyl)-1-nitrosourea, is commercially available as single vials of lyophilized material as BiCNU®, 5-(3,3-dimethyl-1-triazeno)-imidazole-4-carboxamide, is commercially available as single vials of material as DTIC-Dome®. Furthermore, alkylating agents include (a) alkylating-like platinum-based chemotherapeutic agents such as cisplatin, carboplatin, nedaplatin, oxaliplatin, satraplatin, and (SP-4-3)-(cis)-amminedichloro-[2-methylpyridine]platinum(II); (b) alkyl sulfonates such as busulfan; (c) ethyleneimine and methylmelamine derivatives such as altretamine and thiotepa; (d) nitrogen mustards such as chlorambucil, cyclophosphamide, estramustine, ifosfamide, mechlorethamine, trofosamide, prednimustine, melphalan, and uramustine; (e) nitrosoureas such as carmustine, lomustine, fotemustine, nimustine, ranimustine and streptozocin; (f) triazenes and imidazotetrazines such as dacarbazine, procarbazine, temozolamide, and temozolomide. Anti-tumor antibiotics are non-phase specific agents which are believed to bind or intercalate with DNA. This may result in stable DNA complexes or strand breakage, which disrupts ordinary function of the nucleic acids, leading to cell death. Examples of anti-tumor antibiotic agents include, but are not limited to, anthracyclines such as daunorubicin (including liposomal daunorubicin), doxorubicin (including liposomal doxorubicin), epirubicin, idarubicin, and valrubicin; streptomyces-related agents such as bleomycin, actinomycin, mithramycin, mitomycin, porfiromycin; and mitoxantrone. Dactinomycin, also know as Actinomycin D, is commercially available in injectable form as COSMEGEN®. Daunorubicin, (8S-cis+8-acetyl-10-[(3-amino-2,3,6-trideoxy-α-L-lyxohexopyranosyl)oxy]-7,8,9,10-tetrahydro-6,8,11-trihydroxy-1-methoxy-5,12-naphthacenedione hydrochloride, is commercially available as a liposomal injectable form as DAUNOXOME® or as an injectable as CERUBIDINE®. Doxorubicin, (8S, 10S)-10-[(3-amino-2,3,6-trideoxy-α-L-lyxohexopyranosyl)oxy]-8-glycoloyl, 7,8,9,10-tetrahydro-6,8,11-trihydroxy-1-methoxy-5,12-naphthacenedione hydrochloride, is commercially available in an injectable form as RUBEX® or ADRIAMYCIN RDF®. Bleomycin, a mixture of cytotoxic glycopeptide antibiotics isolated from a strain of Streptomyces verticil/us, is commercially available as BLENOXANE®.

Topoisomerase inhibitors include topoisomerase I inhibitors such as camptothecin, topotecan, irinotecan, rubitecan, and belotecan; and topoisomerase II inhibitors such as etoposide, teniposide, and amsacrine.

Topoisomerase II inhibitors include, but are not limited to, epipodophyllotoxins, which are phase specific anti-neoplastic agents derived from the mandrake plant. Epipodophyllotoxins typically affect cells in the S and G2 phases of the cell cycle by forming a ternary complex with topoisomerase II and DNA causing DNA strand breaks. The strand breaks accumulate and cell death follows. Examples of epipodophyllotoxins include, but are not limited to, etoposide, teniposide, and amsacrine. Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Teniposide, 4′-demethyl-epipodophyllotoxin 9[4,6-0-(R)-thenylidene-β-D-glucopyranoside], is commercially available as an injectable solution as VUMON® and is commonly known as VM-26.

Topoisomerase I inhibitors including, camptothecin and camptothecin derivatives. Examples of topoisomerase I inhibitors include, but are not limited to camptothecin, topotecan, irinotecan, rubitecan, belotecan and the various optical forms (i.e., (R), (S) or (R,S)) of 7-(4-methylpiperazino-methylene)-10,11-ethylenedioxy-camptothecin, as described in U.S. Pat. Nos. 6,063,923; 5,342,947; 5,559,235; 5,491,237 and pending U.S. patent application Ser. No. 08/977,217 filed Nov. 24, 1997. Irinotecan HCl, (4S)-4,11-diethyl-4-hydroxy-9-[(4-piperidinopiperidino)-carbonyloxy]-1H-yrano[3′,4′,6,7]indolizino[1,2-b]quinoline-3,14(4H, 12H)-dione hydrochloride, is commercially available as the injectable solution CAMPTOSAR®. Irinotecan is a derivative of camptothecin which binds, along with its active metabolite 8N-38, to the topoisomerase I-DNA complex. Topotecan HCl, (S)-10-[(dimethylamino)methyl]-4-ethyl-4,9-dihydroxy-1H-pyrano[3′,4′,6,7]indolizino[1,2-b]quinoline-3,14-(4H, 12H)-dione monohydrochloride, is commercially available as the injectable solution HYCAMTIN®.

Anti-metabolites include (a) purine analogs such as fludarabine, cladribine, chlorodeoxyadenosine, clofarabine, mercaptopurine, pentostatin, and thioguanine; (b) pyrimidine analogs such as fluorouracil, gemcitabine, capecitabine, cytarabine, azacitidine, edatrexate, floxuridine, and troxacitabine; (c) antifolates, such as methotrexate, pemetrexed, raltitrexed, and trimetrexate. Anti-metabolites also include thyniidylate synthase inhibitors, such as fluorouracil, raltitrexed, capecitabine, floxuridine and pemetrexed; and ribonucleotide reductase inhibitors such as claribine, clofarabine and fludarabine. Antimetabolite neoplastic agents are phase specific anti-neoplastic agents that typically act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Anti-metabolites, include purine analogs, such as fludarabine, cladribine, chlorodeoxyadenosine, clofarabine, mercaptopurine, pentostatin, erythrohydroxynonyladenine, fludarabine phosphate and thioguanine; pyrimidine analogs such as fluorouracil, gemcitabine, capecitabine, cytarabine, azacitidine, edatrexate, floxuridine, and troxacitabine; antifolates, such as methotrexate, pemetrexed, raltitrexed, and trimetrexate. Cytarabine, 4-amino-1-p-D-arabinofuranosyl-2 (1H)-pyrimidinone, is commercially available as CYTOSAR-U® and is commonly known as Ara-C. Mercaptopurine, 1,7-dihydro-6H-purine-6-thione monohydrate, is commercially available as PURINETHOL®. Thioguanine, 2-amino-1,7-dihydro-6H-purine-6-thione, is commercially available as TABLOID®. Gemcitabine, 2′-deoxy-2′,2′-difluorocytidine monohydrochioride (p-isomer), is commercially available as GEMZAR®.

Hormonal therapies include (a) androgens such as fluoxymesterone and testolactone; (b) antiandrogens such as bicalutamide, cyproterone, flutamide, and nilutamide; (c) aromatase inhibitors such as aminoglutethimide, anastrozole, exemestane, formestane, and letrozole; (d) corticosteroids such as dexamethasone and prednisone; (e) estrogens such as diethylstilbestrol; (f) antiestrogens such as fulvestrant, raloxifene, tamoxifen, and toremifine; (g) LHRH agonists and antagonists such as buserelin, goserelin, leuprolide, and triptorelin; (h) progestins such as medroxyprogesterone acetate and megestrol acetate; and (i) thyroid hormones such as levothyroxine and liothyronine. Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, androgens such as fluoxymesterone and testolactone; antiandrogens such as bicalutamide, cyproterone, flutamide, and nilutamide; aromatase inhibitors such as aminoglutethimide, anastrozole, exemestane, formestane, vorazole, and letrozole; corticosteroids such as dexamethasone, prednisone and prednisolone; estrogens such as diethylstilbestrol; antiestrogens such as fulvestrant, raloxifene, tamoxifen, toremifine, droloxifene, and iodoxyfene, as well as selective estrogen receptor modulators (SERMS) such those described in U.S. Pat. Nos. 5,681,835, 5,877,219, and 6,207,716; 5α-reductases such as finasteride and dutasteride; gonadotropin-releasing hormone (GnRH) and analogues thereof which stimulate the release of leutinizing hormone (LH) and/or follicle stimulating hormone (FSH), for example LHRH agonists and antagonists such as buserelin, goserelin, leuprolide, and triptorelin; progestins such as medroxyprogesterone acetate and megestrol acetate; and thyroid hormones such as levothyroxine and liothyronine.

Signal transduction pathway inhibitors are those inhibitors, which block or inhibit a chemical process which evokes an intracellular change, such as cell proliferation or differentiation. Signal tranduction inhibitors useful in the present invention include, e.g., inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Molecular targeted agents include (a) receptor tyrosine kinase (‘RTK’) inhibitors, such as inhibitors of EGFR, including erlotinib, gefitinib, and neratinib; inhibitors of VEGFR including vandetanib, semaxinib, and cediranib; and inhibitors of PDGFR; further included are RTK inhibitors that act at multiple receptor sites such as lapatinib, which inhibits both EGFR and HER2, as well as those inhibitors that act at each of C-kit, PDGFR and VEGFR, including but not limited to axitinib, sunitinib, sorafenib and toceranib; also included are inhibitors of BCR-ABL, c-kit and PDGFR, such as imatinib; (b) FKBP binding agents, such as an immunosuppressive macrolide antibiotic, including bafilomycin, rapamycin (sirolimus) and everolimus; (c) gene therapy agents, antisense therapy agents, and gene expression modulators such as the retinoids and rexinoids, e.g. adapalene, bexarotene, trans-retinoic acid, 9-cis-retinoic acid, and N-(4-hydroxyphenyl)retinamide; (d) phenotype-directed therapy agents, including monoclonal antibodies such as alemtuzumab, bevacizumab, cetuximab, ibritumomab tiuxetan, rituximab, and trastuzumab; (e) immunotoxins such as gemtuzumab ozogamicin; (f) radioimmunoconjugates such as 131I-tositumomab; and (g) cancer vaccines.

Several protein tyrosine kinases catalyse the phosphorylation of specific tyrosyl residues in various proteins involved in the regulation of cell growth. Such protein tyrosine kinases can be broadly classified as receptor or non-receptor kinases. Receptor tyrosine kinases are transmembrane proteins having an extracellular ligand binding domain, a transmembrane domain, and a tyrosine kinase domain. Receptor tyrosine kinases are involved in the regulation of cell growth and are sometimes termed growth factor receptors.

Inappropriate or uncontrolled activation of many of these kinases, for example by over-expression or mutation, has been shown to result in uncontrolled cell growth. Accordingly, the aberrant activity of such kinases has been linked to malignant tissue growth. Consequently, inhibitors of such kinases could provide cancer treatment methods.

Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene.

Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al., Drug Discov. Today (1997), 2(2):50-63; and Lofts, F. J. et al., “Growth factor receptors as targets”, New

Molecular Targets for Cancer Chemotherapy, ed. Workman, Paul and Kerr, David, CRC press 1994, London. Specific examples of receptor tyrosine kinase inhibitors include, but are not limited to, sunitinib, erlotinib, gefitinib, and imatinib.

Tyrosine kinases which are not growth factor receptor kinases are termed non-receptor tyrosine kinases. Non-receptor tyrosine kinases useful in the present invention, which are targets or potential targets of anti-cancer drugs, include cSrc, Lek, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., J. Hematotherapy & Stem Cell Res. (1999) 8(5): 465-80; and Bolen, J. B., Brugge, J .S., Annual Review of Immunology. (1997) 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E., J. Pharmacol. Toxicol. Methods. (1995), 34(3): 125-32. Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., J Biochemistry. (1999) 126 (5): 799-803; Brodt, P, Samani, A, & Navab, R, Biochem. Pharmacol. (2000) 60:1101-1107; Massague, J., Weis-Garcia, F., Cancer Surv. (1996) 27:41-64; Philip, P. A, and Harris, A L, Cancer Treat. Res. (1995) 78: 3-27; Lackey, K. et al. Bioorg. Med. Chem. Letters, (2000) 10(3): 223-226; U.S. Pat. No. 6,268,391; and Martinez-Lacaci, I., et al., Int J. Cancer (2000), 88(1): 44-52. Inhibitors of Phosphotidyl inositol-3 Kinase family members including blockers of P13-kinase, ATM, DNA-PK, and Ku are also useful in the present invention. Such kinases are discussed in Abraham, R T. Current Opin. Immunol. (1996), 8(3): 412-8; Canman, C E., Lim, D. S., Oncogene (1998) 17(25): 3301-8; Jackson, S. P., Int. J. Biochem. Cell Biol. (1997) 29(7):935-8; and Zhong, H. et al., Cancer Res. (2000) 60(6):1541-5. Also useful in the present invention are Myo-inositol signaling inhibitors such as phospholipase C blockers and Myoinositol analogues. Such signal inhibitors are described in Powis, G., and Kozikowski A, (1994) New Molecular Targets for Cancer Chemotherapy, ed., Paul Workman and David Kerr, CRC Press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R, Gervasoni, S I, Matar, P., J. Biomed. Sci. (2000) 7(4): 292-8; Ashby, M. N., Curr. Opin. Lipidol. (1998) 9(2): 99-102; and Oliff, A., Biochim. Biophys. Acta, (1999) 1423(3):C19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al., Cancer Treat. Rev., (2000) 26(4): 269-286); Herceptin® erbB2 antibody (see Stern, D F, Breast Cancer Res. (2000) 2(3):176-183); and 2CB VEGFR2 specific antibody (see Brekken, R. A. et al., Cancer Res. (2000) 60(18):5117-24).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphav beta3) that will inhibit angiogenesis; endostatin and angiostatin (non-RTK) may also prove useful in combination with the disclosed erb family inhibitors. (See Bruns, C J et al., Cancer Res. (2000), 60(11): 2926-2935; Schreiber A B, Winkler M E, & Derynck R., Science (1986) 232(4755):1250-53; Yen L. et al., Oncogene (2000) 19(31): 3460-9).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T, et al., Cancer Res. (2000) 60(13):3569-76; and Chen Y, et al., Cancer Res. (1998) 58(9):1965-71.

Agents used in pro-apoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family. Therefore, strategies designed to downregulate the expression of bcl-2 in tumors have demonstrated clinical benefit and are now in Phase II/III trials, namely Genta's G3139 bcl-2 antisense oligonucleotide. Such pro-apoptotic strategies using the antisense oligonucleotide strategy for bcl-2 are discussed in Waters J S, et al., J. Clin. Oncol. (2000) 18(9): 1812-23; and Kitada S, et al. Antisense Res. Dev. (1994) 4(2): 71-9.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania G R & Chang Y-T., Exp. Opin. Thee. Patents (2000) 10(2):215-30.

Other molecular targeted agents include FKBP binding agents, such as the immunosuppressive macrolide antibiotic, rapamycin; gene therapy agents, antisense therapy agents, and gene expression modulators such as the retinoids and rexinoids, e.g. adapalene, bexarotene, trans-retinoic acid, 9-cisretinoic acid, and N-(4 hydroxyphenyl)retinamide; phenotype-directed therapy agents, including: monoclonal antibodies such as alemtuzumab, bevacizumab, cetuximab, ibritumomab tiuxetan, rituximab, and trastuzumab; immunotoxins such as gemtuzumab ozogamicin, radioimmunoconjugates such as 131-tositumomab; and cancer vaccines.

Anti-tumor antibiotics include (a) anthracyclines such as daunorubicin (including liposomal daunorubicin), doxorubicin (including liposomal doxorubicin), epirubicin, idarubicin, and valrubicin; (b) streptomyces-related agents such as bleomycin, actinomycin, mithramycin, mitomycin, porfiromycin; and (c) anthracenediones, such as mitoxantrone and pixantrone. Anthracyclines have three mechanisms of action: intercalating between base pairs of the DNA/RNA strand; inhibiting topoiosomerase II enzyme; and creating iron-mediated free oxygen radicals that damage the DNA and cell membranes. Anthracyclines are generally characterized as topoisomerase II inhibitors.

Monoclonal antibodies include, but are not limited to, murine, chimeric, or partial or fully humanized monoclonal antibodies. Such therapeutic antibodies include, but are not limited to antibodies directed to tumor or cancer antigens either on the cell surface or inside the cell. Such therapeutic antibodies also include, but are not limited to antibodies directed to targets or pathways directly or indirectly associated with CK2. Therapeutic antibodies may further include, but are not limited to antibodies directed to targets or pathways that directly interact with targets or pathways associated with the compounds of the present invention. In one variation, therapeutic antibodies include, but are not limited to anticancer agents such as Abagovomab, Adecatumumab, Afutuzumab, Alacizumab pegol, Alemtuzumab, Altumomab pentetate, Anatumomab mafenatox, Apolizumab, Bavituximab, Belimumab, Bevacizumab, Bivatuzumab mertansine, Blinatumomab, Brentuximab vedotin, Cantuzumab mertansine, Catumaxomab, Cetuximab, Citatuzumab bogatox, Cixutumumab, Clivatuzumab tetraxetan, Conatumumab, Dacetuzumab, Detumomab, Ecromeximab, Edrecolomab, Elotuzumab, Epratuzumab, Ertumaxomab, Etaracizumab, Farletuzumab, Figitumumab, Fresolimumab, Galiximab, Glembatumumab vedotin, Ibritumomab tiuxetan, Intetumumab, Inotuzumab ozogamicin, Ipilimumab, Iratumumab, Labetuzumab, Lexatumumab, Lintuzumab, Lucatumumab, Lumiliximab, Mapatumumab, Matuzumab, Milatuzumab, Mitumomab, Nacolomab tafenatox, Naptumomab estafenatox, Necitumumab, Nimotuzumab, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Sibrotuzumab, Tacatuzumab tetraxetan, Taplitumomab paptox, Tenatumomab, Ticilimumab, Tigatuzumab, Tositumomab, Trastuzumab, Tremelimumab, Tucotuzumab celmoleukin, Veltuzumab, Volociximab, Votumumab, Zalutumumab, and Zanolimumab. In some embodiments, such therapeutic antibodies include, alemtuzumab, bevacizumab, cetuximab, daclizumab, gemtuzumab, ibritumomab tiuxetan, pantitumumab, rituximab, tositumomab, and trastuzumab; in other embodiments, such monoclonal antibodies include alemtuzumab, bevacizumab, cetuximab, ibritumomab tiuxetan, rituximab, and trastuzumab; alternately, such antibodies include daclizumab, gemtuzumab, and pantitumumab. In yet another embodiment, therapeutic antibodies useful in the treatment of infections include but are not limited to Afelimomab, Efungumab, Exbivirumab, Felvizumab, Foravirumab, Ibalizumab, Libivirumab, Motavizumab, Nebacumab, Pagibaximab, Palivizumab, Panobacumab, Rafivirumab, Raxibacumab, Regavirumab, Sevirumab, Tefibazumab, Tuvirumab, and Urtoxazumab. In a further embodiment, therapeutic antibodies can be useful in the treatment of inflammation and/or autoimmune disorders, including, but are not limited to, Adalimumab, Atlizumab, Atorolimumab, Aselizumab, Bapineuzumab, Basiliximab, Benralizumab, Bertilimumab, Besilesomab, Briakinumab, Canakinumab, Cedelizumab, Certolizumab pegol, Clenoliximab, Daclizumab, Denosumab, Eculizumab, Edobacomab, Efalizumab, Erlizumab, Fezakinumab, Fontolizumab, Fresolimumab, Gantenerumab, Gavilimomab, Golimumab, Gomiliximab, Infliximab, Inolimomab, Keliximab, Lebrikizumab, Lerdelimumab, Mepolizumab, Metelimumab, Muromonab-CD3, Natalizumab, Ocrelizumab, Odulimomab, Omalizumab, Otelixizumab, Pascolizumab, Priliximab, Reslizumab, Rituximab, Rontalizumab, Rovelizumab, Ruplizumab, Sifalimumab, Siplizumab, Solanezumab, Stamulumab, Talizumab, Tanezumab, Teplizumab, Tocilizumab, Toralizumab, Ustekinumab, Vedolizumab, Vepalimomab, Visilizumab, Zanolimumab, and Zolimomab aritox. In yet another embodiment, such therapeutic antibodies include, but are not limited to adalimumab, basiliximab, certolizumab pegol, eculizumab, efalizumab, infliximab, muromonab-CD3, natalizumab, and omalizumab. Alternately the therapeutic antibody can include abciximab or ranibizumab. Generally a therapeutic antibody is non-conjugated, or is conjugated with a radionuclide, cytokine, toxin, drug-activating enzyme or a drug-filled liposome.

Akt inhibitors include 1L6-Hydroxymethyl-chiro-inositol-2-(R)-2-O-methyl-3-O-octadecyl-sn-glycerocarbonate, SH-5 (Calbiochem Cat. No. 124008), SH-6 (Calbiochem Cat. No. Cat. No. 124009), Calbiochem Cat. No. 124011, Triciribine (NSC154020, Calbiochem Cat. No. 124012), 10-(4′-(N-diethylamino)butyl)-2-chlorophenoxazine, Cu(II)Cl₂(3-Formylchromone thiosemicarbazone), 1,3-dihydro-1-(1-(4-(6-phenyl-1H-imidazo[4,5-g]quinoxalin-7-yl)phenyl)methyl)-4-piperidinyl)-2H-benzimidazol-2-one, GSK690693 (4-(2-(4-amino-1,2,5-oxadiazol-3-yl)-1-ethyl-7-{[(3S)-3-piperidinylmethyl]oxy}-1H-imidazo[4,5-c]pyridin-4-yl)-2-methyl-3-butyn-2-ol), SR 13668 ((2,10-dicarbethoxy-6-methoxy-5,7-dihydro-indolo[2,3-b]carbazole), GSK2141795, Perifosine, GSK21110183, XL418, XL147, PF-04691502, BEZ-235 [2-Methyl-2-[4-(3-methyl-2-oxo-8-quinolin-3-yl-2,3-dihydro-imidazo[4,5-c]quinolin-1-yl)-phenyl]-propionitrile], PX-866 ((acetic acid (1S,4E,10R,11R,13S,14R)-[4-diallylaminomethylene-6-hydroxy-1-methoxymethyl-10,13-dimethyl-3,7,17-trioxo-1,3,4,7,10,11,12,13,14,15,16,17-dodecahydro-2-oxa-cyclopenta[a]phenanthren-11-yl ester)), D-106669, CAL-101, GDC0941 (2-(1H-indazol-4-yl)-6-(4-methanesulfonyl-piperazin-1-ylmethyl)-4-morpholin-4-yl-thieno[3,2-d]pyrimidine), SF1126, SF1188, SF2523, TG100-115 [3-[2,4-diamino-6-(3-hydroxyphenyl)pteridin-7-yl]phenol]. A number of these inhibitors, such as, for example, BEZ-235, PX-866, D 106669, CAL-101, GDC0941, SF1126, SF2523 are also identified in the art as PI3K/mTOR inhibitors; additional examples, such as PI-103 [3-[4-(4-morpholinylpyrido[3′,2′:4,5]furo[3,2-d]pyrimidin-2-yl]phenol hydrochloride] are well-known to those of skill in the art. Additional well-known, PI3K inhibitors include LY294002 [2-(4-morpholinyl)-8-phenyl-4H-1-benzopyran-4-one] and wortmannin. mTOR inhibitors known to those of skill in the art include temsirolimus, deforolimus, sirolimus, everolimus, zotarolimus, and biolimus A9. A representative subset of such inhibitors includes temsirolimus, deforolimus, zotarolimus, and biolimus A9.

HDAC inhibitors include (i) hydroxamic acids such as Trichostatin A, vorinostat (suberoylanilide hydroxamic acid (SAHA)), panobinostat (LBH589) and belinostat (PXD101) (ii) cyclic peptides, such as trapoxin B, and depsipeptides, such as romidepsin (NSC630176), (iii) benzamides, such as MS-275 (3-pyridylmethyl-N-{4-[(2-aminophenyl)-carbamoyl]-benzyl}-carbamate), CI994 (4-acetylamino-N-(2aminophenyl)-benzamide) and MGCD0103 (N-(2-aminophenyl)-4-[4-(pyridin-3-yl)pyrimidin-2-ylamino)methyl)benzamide), (iv) electrophilic ketones, (v) the aliphatic acid compounds such as phenylbutyrate and valproic acid. Hsp90 inhibitors include benzoquinone ansamycins such as geldanamycin, 17-DMAG (17-Dimethylamino-ethylamino-17-demethoxygeldanamycin), tanespimycin (17-AAG, 17-allylamino-17-demethoxygeldanamycin), ECS, retaspimycin (IPI-504, 18,21-didehydro-17-demethoxy-18,21-dideoxo-18,21-dihydroxy-17-(2-propenylamino)-geldanamycin), and herbimycin; pyrazoles such as CCT 018159 (4-[4-(2,3-dihydro-1,4-benzodioxin-6-yl)-5-methyl-1H-pyrazol-3-yl]-6-ethyl-1,3-benzenediol); macrolides, such as radicocol; as well as BIIB021 (CNF2024), SNX-5422, STA-9090, and AUY922.

Miscellaneous agents include altretamine, arsenic trioxide, gallium nitrate, hydroxyurea, levamisole, mitotane, octreotide, procarbazine, suramin, thalidomide, lenalidomide, photodynamic compounds such as methoxsalen and sodium porfimer, and proteasome inhibitors such as bortezomib.

Biologic therapy agents include: interferons such as interferon-α2a and interferon-α2b, and interleukins such as aldesleukin, denileukin diftitox, and oprelvekin.

In addition to these anticancer agents intended to act against cancer cells, combination therapies including the use of protective or adjunctive agents, including: cytoprotective agents such as armifostine, dexrazonxane, and mesna, phosphonates such as parmidronate and zoledronic acid, and stimulating factors such as epoetin, darbepoetin, filgrastim, PEG-filgrastim, and sargramostim, are also envisioned.

Thus in one aspect, the invention provides a method to treat a condition described herein using a compound of the invention in combination therapy with any of the foregoing additional therapeutic agents and inhibitors and the like. The method comprises administering a compound of the invention to a subject in need thereof, and an additional agent selected from the agents and inhibitors disclosed above, wherein the combined amounts of the compound of the invention and of the additional therapeutic agent are effective to treat the cell proliferative condition. The invention further provides pharmaceutical compositions comprising at least one compound of the invention, i.e., a compound of the invention as described herein, admixed with at least one additional therapeutic agent selected from the foregoing agents and inhibitors. Optionally, these pharmaceutical compositions further comprise at least one pharmaceutically acceptable excipient.

### EXAMPLES

The compounds of the invention as described above can be synthesized using methods, techniques, and materials known to those of skill in the art, such as described, for example, in March, ADVANCED ORGANIC CHEMISTRY 4.sup.th Ed., (Wiley 1992); Carey and Sundberg, ADVANCED ORGANIC CHEMISTY 3.sup.rd Ed., Vols. A and B (Plenum 1992), and Green and Wuts, PROTECTIVE GROUPS IN ORGANIC SYNTHESIS 2.sup.nd Ed. (Wiley 1991). Starting materials useful for preparing compounds of the invention and intermediates thereof are commercially available from sources, such as Aldrich Chemical Co. (Milwaukee, Wis.), Sigma Chemical Co. (St. Louis, Mo.), Maybridge (Cornwall, England), Asinex (Winston-Salem, N.C.), ChemBridge (San Diego, Calif.), ChemDiv (San Diego, Calif.), SPECS (Delft, The Netherlands), Timtec (Newark, D.E.), or alternatively can be prepared by well-known synthetic methods (see, e.g., Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-21, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” 3d Edition, John Wiley & Sons, 1995). Other methods for synthesis of the present compounds and/or starting materials thereof are either described in the art or will be readily apparent to the skilled artisan. Alternatives to the reagents and/or protecting groups may be found in the references provided above and in other compendiums well known to the skilled artisan.

Preparation of the present compounds may include one or more steps of protection and deprotection (e.g., the formation and removal of acetal groups). Guidance for selecting suitable protecting groups can be found, for example, in Greene & Wuts, “Protective Groups in Organic Synthesis,” Wiley Interscience, 1999. In addition, the preparation may include various purifications, such as column chromatography, flash chromatography, thin-layer chromatography (TLC), recrystallization, distillation, high-pressure liquid chromatography (HPLC) and the like. Also, various techniques well known in the chemical arts for the identification and quantification of chemical reaction products, such as proton and carbon-13 nuclear magnetic resonance (¹H and ¹³C NMR), infrared and ultraviolet spectroscopy (IR and UV), X-ray crystalography, elemental analysis (EA), HPLC and mass spectroscopy (MS) can be used as well. The preparation may also involve any other methods of protection and deprotection, purification and identification and quantification that are well known in the chemical arts.

In some embodiments, the deuterated-compounds can be prepared by modifying the synthesis of the corresponding undeuterated compounds. In some embodiments, certain starting materials or chemical reagents in the synthesis of the corresponding undeuterated compounds can be replaced with the deuterated starting materials or chemical reagents to make the deuterated compounds. For example, certain deuterated compounds and reagents can be purchased from Aldrich Chemicals, Cambridge Isotope Laboratories, or C/D/N Isotopes. In some specific embodiments, deuterated starting materials or chemical reagents can be prepared by transformation of the undeuterated precursors. One common method of preparing deuterated compounds is by reduction of certain undeuterated precursors using deuterated reductive agents.

Synthesis of the examples of the deuterated compounds are illustrated in the general Schemes 1 and 2 below. One skilled in the art can readily derive the synthesis of the present deuterated compounds from the following examples according to the methods discussed above.

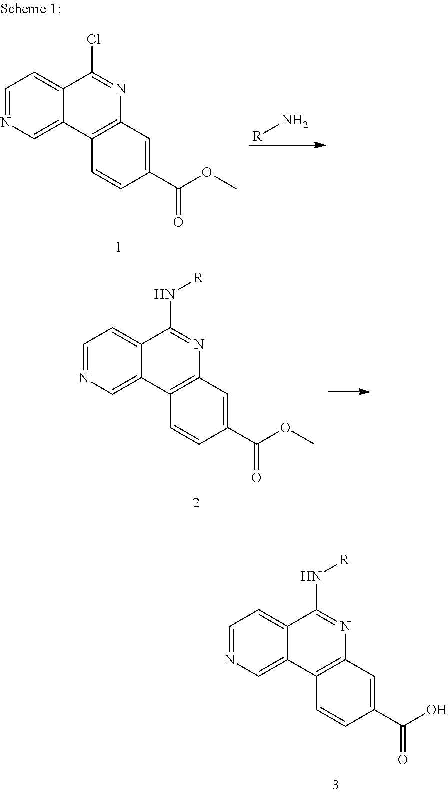

Additional descriptions about synthetic method to prepare Compounds 1, 2 and 3 can be found in U.S. Utility application Ser. No. 11/849,230, filed on Aug. 31, 2007 and published as US 2009/0105233 A1 on Apr. 23, 2009, the contents of which are hereby incorporated by reference in their entirety. Representative examples of R—NH₂ from commercial sources are exemplified as follows:

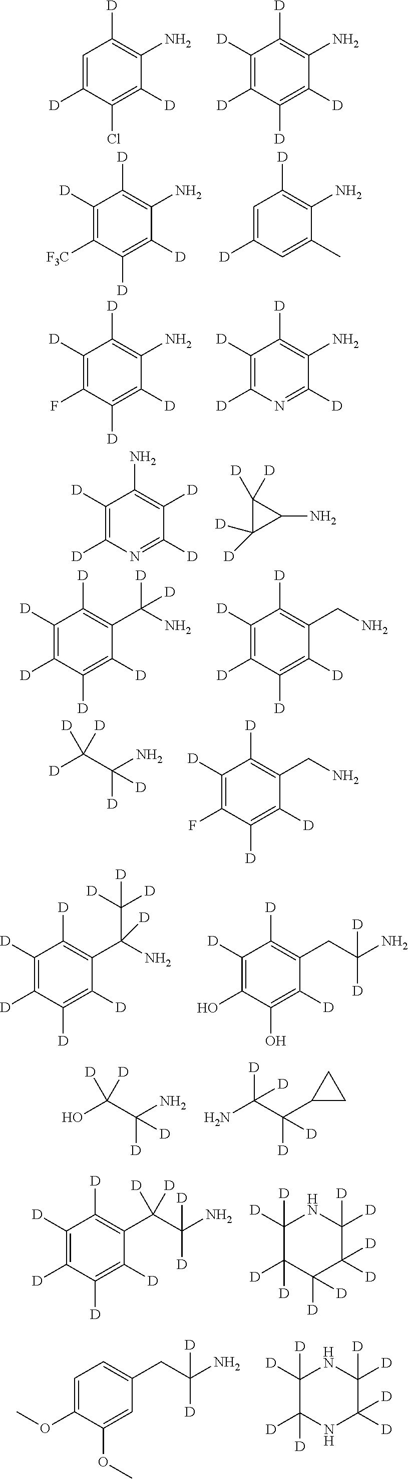

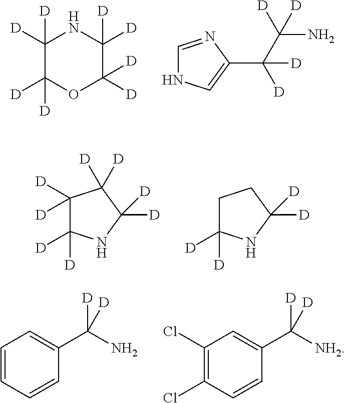

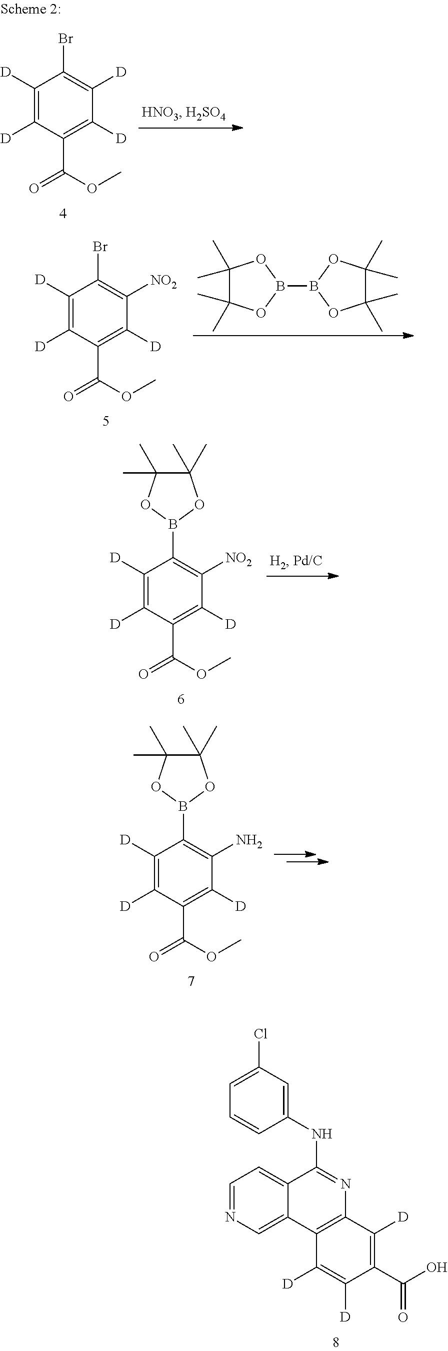

Preparation of the present compounds can also be carried out as described in Scheme 2. The nitration of reagent 4 can lead to reagent 5. The synthesis of boronic ester 6 can be carried out by the treatment of a compound of formula 5 with bis (pinacolato) diboron and a palladium (0) source in appropriate solvent and temperature. Suitable sources of palladium (0) include but are not limited to palladium (II) acetate and tris(dibenzyldeneacetone) dipalladium (0). The reduction of the nitro group in compound of formula 6 with hydrogen in the presence of Pd/C and appropriate solvent can produce compound of formula 7. Compound of formula 8 can be achieved as described in the 32.00 application.

### Example 1

### Modulation of CK2 Activity in Cell-Free In Vitro Assays

Modulatory activity of the present compounds can be assessed in vitro in cell-free CK2 assays. Test compounds in aqueous solution are added at a volume of 10 microliters, to a reaction mixture comprising 10 microliters Assay Dilution Buffer (ADB; 20 mM MOPS, pH 7.2, 25 mM beta-glycerolphosphate, 5 mM EGTA, 1 ‘mM sodium orthovanadate and 1 mM dithiothreitol), 10 microliters of substrate peptide (RRRDDDSDDD, dissolved in ADB at a concentration of 1 mM), 10 microliters of recombinant human CK2 (25 ng dissolved in ADB; Upstate). Reactions are initiated by the addition of 10 microliters of ATP Solution (90% 75 mM MgCl₂, 75 micromolar ATP dissolved in ADB; 10% [γ-³³P]ATP (stock 1 mCi/100 μl; 3000 Ci/mmol (Perkin Elmer) and maintained for 10 minutes at 30 degrees C. The reactions are quenched with 100 microliters of 0.75% phosphoric acid, then transferred to and filtered through a phosphocellulose filter plate (Millipore). After washing each well 5 times with 0.75% phosphoric acid, the plate is dried under vacuum for 5 min and, following the addition of 15 μl of scintilation fluid to each well, the residual radioactivity is measured using a Wallac luminescence counter.

### Example 2

### Cell Proliferation Modulatory Activity

A representative cell-proliferation assay protocol using Alamar Blue dye (stored at 4° C., use 20 μl per well) is described hereafter.

### 96-Well Plate Setup and Compound Treatment

a. Split and trypsinize cells.

b. Count cells using hemocytometer.

c. Plate 4,000-5,000 cells per well in 100 μl of medium and seed into a 96-well plate according to the following plate layout. Add cell culture medium only to wells B10 to B12. Wells B1 to B9 have cells but no compound added.

| | | | | |
|---|---|---|---|---|
| 1 | 2 4 | 5 7 | 8 10 | 11 |
| 3 | 6 | 9 | 12 | |

| A | EMPTY | | |
|---|---|---|---|
| B | NO | COMPOUND | Medium |
| | ADDED | | Only |

| | | | | | |
|---|---|---|---|---|---|
| C | 10 nM | 100 nM | 1 uM | 10 uM | Control |
| D | 10 nM | 100 nM | 1 uM | 10 uM | Compound |
| E | 10 nM | 100 nM | 1 uM | 10 uM | Compound |
| F | 10 nM | 100 nM | 1 uM | 10 uM | Compound |
| G | 10 nM | 100 nM | 1 uM | 10 uM | Compound |
| H | EMPTY | | | | |

d. Add 100 μl of 2× drug dilution to each well in a concentration shown in the plate layout above. At the same time, add 100 μl of media into the control wells (wells B10 to B 12). Total volume is 200 μl /well.
e. Incubate four (4) days at 37° C., 5% CO2 in a humidified incubator.
f. Add 20 μl Alamar Blue reagent to each well.
g. Incubate for four (4) hours at 37° C., 5% CO2 in a humidified incubator.
h. Record fluorescence at an excitation wavelength of 544 nm and emission wavelength of 590 nm using a microplate reader.

In the assays, cells are cultured with a test compound for approximately four days, the dye then is added to the cells and fluorescence of non-reduced dye is detected after approximately four hours. Different types of cells can be utilized in the assays (e.g., HCT-116 human colorectal carcinoma cells, PC-3 human prostatic cancer cells and MiaPaca human pancreatic carcinoma cells). Anti-proliferative effects of representative compounds are provided hereafter.

### Example 3

### Modulation of Endogenous CK2 Activity

The human leukemia Jurkat T-cell line is, maintained in RPMI 1640 (Cambrex) supplemented with 10% fetal calf serum and 50 ng/ml Geutamycin. Before treatment cells are ished, resuspended at a density of about 10⁶ cells/milliliter in medium containing 1% fetal calf serum and incubated in the presence of indicated mounts of drug for two hours. Cells are recovered by centrifugation, lysed using a hypotonic buffer (20 mM Tris/HCl pH 7.4; 2 mM EDTA; 5 mM EGTA; 10 mM mercaptoethanol; 10 mM NaF; 1 uM Okadaic acid; 10% v/v glycerol; 0.05% NP-40; 1% Protease Inhibitor Cocktail) and protein from the cleared lysate is diluted to 1 microgram per microliter in Assay Dilution Buffer (ADB; 20 mM MOPS, pH 7.2, 25 mM β-glycerolphosphate, 5 mM EGTA, 1 mM sodium orthovanadate and 1 mM dithiothreitol). To 20 microliters of diluted protein is added 10 microliters of substrate peptide (RRRDDDSDDD, dissolved in ADB at a concentration of 1 mM) and 10 microliters of PKA Inhibitor cocktail (Upstate). Reactions are initiated by the addition of 10 microliters of ATP Solution (90% 75 mM MgCl₂, 100 uM ATP dissolved in ADB; 10% [gamma-³³]ATP (stock 1 mCi/100 microliters; 3000 Ci/mmol (Perkin Elmer)) and maintained for 15 min at 32 degrees C. The reactions are quenched with 100 microliters of 0.75% phosphoric acid, then transferred to and filtered through a phosphocellulose filter plate (Millipore). After washing each well 5 times with 0.75% phosphoric acid, the residual radioactivity is measured using a Wallac luminescence counter.

### Example 4

### Evaluation of Pharmacokinetic Properties

The pharmacokinetics properties of drugs can be investigated in ICR mice following an intravenous (IV) bolus and oral (PO) doses of drug at 5 mg/kg and 25 mg/kg respectively. Blood samples are collected at predetermined times and the plasma separated. Plasma is separated from the blood samples collected at 5, 15 and 30 minutes and 1, 2, 4, 8 and 24 hours post-dose. Drug levels are quantified by the LC/MS/MS method described below. Noncompartmental pharmacokinetic analysis is applied for intravenous administration. A linear trapezoidal rule is used to compute AUC(0-24). The terminal t_{1/2} and C₀ are calculated using the last three and the first three data points, respectively

Bioanalysis is performed using a Quattro Micro LC/MS/MS instrument in the MRM detection mode, with an internal standard (IS). Briefly, 15 μL plasma samples are prepared for analysis using protein precipitation with 120 μL of acetonitrile. The supernatants are transferred into a 96 well plate and subjected to LC-MS/MS analysis using a Phenomenex Polar-RP HPLC column. The mobile phases are 10 mM NH₄HCO₃ in water (Solution-A) and 10 mM NH₄HCO₃ in methanol (Solution-B). The column is initially equilibrated with 25% Solution-B and followed with 100% Solution B over 5 minutes. The method had a dynamic range from 1 to 10,000 ng/mL. Quantitation of the analytes is performed in the batch mode with two bracketing calibration curves according to the bioanalytical sample list.

### Example 5

### Evaluation of Compound Efficacy in Tumor Suppression

The in vivo activity of the present compounds (as referenced as Compound below) can be assessed by intravenous and oral administration to tumor-bearing xenograft mice. The in vivo experiments follow protocols approved by the Animal Use and Care Committee. Female NCr nu/nu mice are purchased from Taconic Farms and group housed in a ventilated rack system on a 12/12 light cycle. All housing materials and water are autoclaved prior to use. The mice are fed ad libitum with gamma irradiated laboratory chow and acidified water. Animals are handled under laminar-flow hoods.

Tumor size (mm³) is calculated using the formula (l×w²)/2, where w=width and l=length in mm of the tumor. Tumor weight is estimated with the assumption that 1 mg is equivalent to 1 mm³ of tumor volume.

For intravenous administration of Compound, animals are inoculated subcutaneously in the right flank with 5×10⁶ MiaPaca cells. Tumors are monitored twice weekly and then daily as they approached the appropriate size for study. On Day 1 of the study, the animals are randomized into n=5 treatment groups with group mean tumor sizes of 160 mm³.

| | | | |
|---|---|---|---|
| Grp 1 | Mean | 160.966 | UTC |
| Grp 2 | Mean | 161.816 | Gemzar |
| Grp 3 | Mean | 161.807 | 30 mg/kg CK2 Compound |
| Grp 4 | Mean | 159.621 | 60 mg/kg CK2 Compound |
| % Dif. | 1.363 | | |
| SD | 1.034. | | |

Animals receive 14 doses of Vehicle, Gemzar at 100 mg/kg Q3D or Compound at either 30 mg/kg or 60 mg/kg by QD intravenous administration. Tumor volume measurements and body weight are recorded on days 3, 6, 8, 10, 13 and 15. Photographs of specific untreated control animals and animals administered 60 mg/kg Compound can be shown in figures.

Compound also is administered orally to MiaPaca xenograft animals and inhibited tumor growth. Compound is formulated as a sodium salt at 10 mg/mL with 2% PEG 300 and buffered to pH 8.4 using sodium phosphate buffer. Compound when administered orally to the animals at a dose of 100 mg/kg QD×8 and then 200 mg/kg QD×5 significantly inhibited tumor growth relative to an untreated control group. Gemzar™ administered at a dose of 80 mg/kg IP Q3D is used as a positive control. Compound also is delivered by oral administration at 100 mg/kg to animals bearing MCF-7 xenografts and at 150 mg/kg to animals bearing PC-3 xenografts, and in both sets of studies, significantly inhibited tumor growth.

It also is determined that Compound reduced CK2 activity in tumors. Assessment of CK2 activity in tumors revealed that tumors from animals treated with Compound had about 40% of the CK2 activity of tumors from animals not treated with Compound or treated with Gemzar™.

The distribution of Compound in the plasma and tumors of animals is assessed.

Caspase staining also is assessed as a biomarker for Compound treatment of tumors. In animals treated with 60 mg/kg of Compound by IV administration, caspase-3 cell staining levels are four-fold greater than in untreated control cells.

### Example 6

### Evaluation of Angiogenesis Inhibition by Endothelial Tube Formation Assay

A human endothelial tube formation assay can be performed using the 96-well BD BioCoat™ Angiogenesis System from BD Biosciences, using the manufacturer's recommended protocol.

Briefly, HUVEC cells (from ATCC) are suspended in 150 ul of media containing 10% FBS at 4×10⁵ cells/ml in each of the 96-wells of the matrigel coated plate in the presence or absence of various concentrations of compound A2. The plate is incubated for 18 hrs at 37° C. The cells are stained with calcein AM and the results visualized by fluorescent microscopy or by phase contrast. It is observed that compound A2 inhibited tube formation in the assay described above over a concentration range of 1 to 5 μM

1. A compound having structural Formula (A): 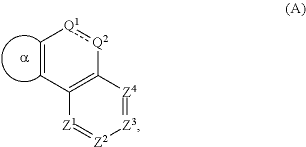
or a pharmaceutically acceptable salt, solvate, and/or prodgug thereof;
wherein the ring labeled α represents a 5 or 6 membered aromatic or heteroaromatic ring fused onto the ring containing Q¹, wherein a is a 6-membered aryl ring optionally containing one or more nitrogen atoms as ring members, or a 5-membered aryl ring selected from thiophene and thiazole; and the ring labeled α optionally contains one or more carbon-bound deuterium;
Q¹ is C═X, Q² is NR⁵, and the bond between Q¹ and Q² is a single bond; or Q¹ is C—X—R⁵, Q² is N, and the bond between Q¹ and Q² is a double bond; and
wherein X represents O, S or NR⁴;
each Z¹, Z², Z³, and Z⁴ is N or CR³ and one or more of Z¹, Z², Z³, and Z⁴ is CR³;
each R³ is independently H, deuterium, or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C12 heteroaryl, deuterated-C7-C12 arylalkyl, or deuterated-C6-C12 heteroarylalkyl group,
or each R³ can be halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H, deuterium, C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C10 heteroaryl, deuterated-C7-C12 arylalkyl, or deuterated-C6-C12 heteroarylalkyl,
and wherein two R on the same atom or on adjacent atoms can be linked to form a 3 to 8 membered ring, optionally containing one or more N, O or S; and the 3 to 8 membered ring optionally contains one or more carbon-bound deuterium;
and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, deuterium, C1-C6 alkyl, C2-C6 heteroalkyl, C1-C6 acyl, C2-C6 heteroacyl, C6-C10 aryl, C5-C10 heteroaryl, C7-12 arylalkyl, C6-12 heteroarylalkyl, deuterated-C1-C6 alkyl, deuterated-C2-C6 heteroalkyl, deuterated-C1-C6 acyl, deuterated-C2-C6 heteroacyl, deuterated-C6-C10 aryl, deuterated-C5-C10 heteroaryl, deuterated-C7-12 arylalkyl, or deuterated-C6-12 heteroarylalkyl; each of which is optionally substituted with one or more groups selected from halo, C1-C4 alkyl, C1-C4 heteroalkyl, C1-C6 acyl, C1-C6 heteroacyl, hydroxy, amino, deuterated-C1-C4 alkyl, deuterated-C1-C4 heteroalkyl, deuterated-C1-C6 acyl, deuterated-C1-C6 heteroacyl, deuterated-hydroxy, deuterated-amino, and ═O;
and wherein two R′ can be linked to form a 3 to 7 membered ring optionally containing up to three heteroatoms selected from N, O and S;
and the 3 to 7 membered ring optionally contains one or more carbon-bound deuterium;
R⁴ is H, deuterium, or optionally substituted member selected from the group consisting of C₁-C₆ alkyl, C2-C6 heteroalkyl, C1-C6 acyl, deuterated-C₁-C₆ alkyl, deuterated-C2-C6 heteroalkyl, and deuterated-C1-C6 acyl;
each R⁵ is independently H, deuterium, or an optionally substituted member selected from the group consisting of C₁₋₁₀ alkyl, C₂₋₁₀ alkenyl, C₂₋₁₀ heteroalkyl, C₃₋₈ carbocyclic ring, C₃₋₈ heterocyclic ring, deuterated-C₁₋₁₀ alkyl, deuterated-C₂₋₁₀ alkenyl, deuterated-C₂₋₁₀ heteroalkyl, deuterated-C₃₋₈ carbocyclic ring, and deuterated-C₃₋₈ heterocyclic ring optionally fused to an additional optionally substituted carbocyclic, heterocyclic, deuterated-carbocyclic, deuterated-heterocyclic ring; or R⁵ is a C₁₋₁₀ alkyl, C₂₋₁₀ alkenyl, C₂₋₁₀ heteroalkyl, deuterated-C₁₋₁₀ alkyl, deuterated-C₂₋₁₀ alkenyl, or deuterated-C₂₋₁₀ heteroalkyl substituted with an optionally substituted C₃₋₈ carbocyclic ring, C₃₋₈ heterocyclic ring, deuterated-C₃₋₈ carbocyclic ring, or deuterated-C₃₋₈ heterocyclic ring; and
in each —NR⁴R⁵, R⁴ and R⁵ together with N may form an optionally substituted 3 to 8 membered ring, which may optionally contain an additional heteroatom selected from N, O and S as a ring member; and the 3 to 8 membered ring optionally contains one or more carbon-bound deuterium; and
with the following provisos:
(a) the compound of Formula (A) comprises at least one carbon-bound deuterium; and
(b) when Q¹ in Formula (A) is C—NHΦ, where Φ is optionally substituted phenyl:
if the ring labeled α is a six-membered ring containing at least one N as a ring member, at least one R³ present must be a polar substituent, or if each R³ is H, then Φ must be substituted; and
if the ring labeled α is phenyl, and three of Z¹ to Z⁴ represent CH, then Z² cannot be C—OR″, and Z³ cannot be NH₂, NO₂, NHC(═O)R″ or NHC(═O)—OR″, where R″ is C1-C4 alkyl.

2. The compound of claim 1, having a structural Formula I, II, III or IV: 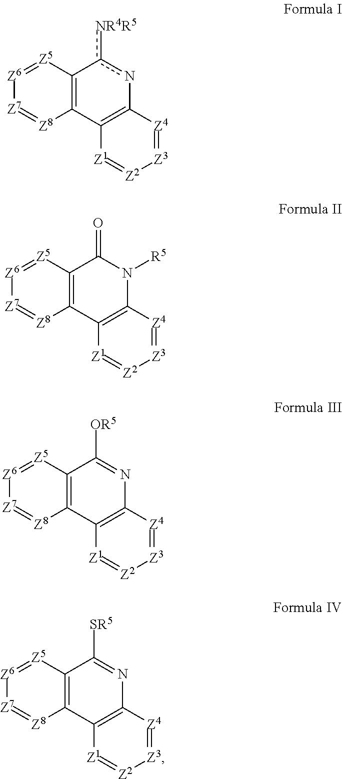
or a pharmaceutically acceptable salt, solvate, and/or prodrug thereof;
wherein:
each Z¹, Z², Z³, and Z⁴ is N or CR³;
each of Z⁵, Z⁶, Z⁷ and Z⁸ is N or CR⁶;
none, one or two of Z¹ to Z⁴ are N and none, one or two of Z⁵—Z⁸ are N;
each R³ and each R⁶ is independently H, deuterium, or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C12 heteroaryl, deuterated-C7-C12 arylalkyl, or deuterated-C6-C12 heteroarylalkyl group,
or each R³ and each R⁶ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, polar substituent, carboxy bioisostere, COOH, COOD, or NO₂,
wherein each R is independently H, deuterium, or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C10 heteroaryl, deuterated-C7-C12 arylalkyl, or deuterated-C6-C12 heteroarylalkyl,
and wherein two R on the same atom or on adjacent atoms can be linked to form a 3 to 8 membered ring, optionally containing one or more N, O or S; and the 3 to 8 membered ring optionally contains one or more carbon-bound deuterium;
and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, deuterium, C1-C6 alkyl, C2-C6 heteroalkyl, C1-C6 acyl, C2-C6 heteroacyl, C6-C10 aryl, C5-C10 heteroaryl, C7-12 arylalkyl, C6-12 heteroarylalkyl, deuterated-C1-C6 alkyl, deuterated-C2-C6 heteroalkyl, deuterated-C1-C6 acyl, deuterated-C2-C6 heteroacyl, deuterated-C6-C10 aryl, deuterated-C5-C10 heteroaryl, C7-12 arylalkyl, or deuterated-C6-12 heteroarylalkyl each of which is optionally substituted with one or more groups selected from halo, C1-C4 alkyl, C1-C4 heteroalkyl, C1-C6 acyl, C1-C6 heteroacyl, hydroxy, amino, deuterated-C1-C4 alkyl, deuterated-C1-C4 heteroalkyl, deuterated-C1-C6 acyl, deuterated-C1-C6 heteroacyl, deuterated-hydroxy, deuterated-amino, and ═O;
and wherein two R′ can be linked to form a 3 to 7 membered ring optionally containing up to three heteroatoms selected from N, O and S;
and the 3 to 7 membered ring optionally contains one or more carbon-bound deuterium;
R⁴ is H or an optionally substituted member selected from the group consisting of C1-C6 alkyl, C2-C6 heteroalkyl, C1-C6 acyl, deuterated-C1-C6 alkyl, deuterated-C2-C6 heteroalkyl, and deuterated-C1-C6 acyl;
each R⁵ is independently H or an optionally substituted member selected from the group consisting of C₁₋₁₀ alkyl, C₂₋₁₀ alkenyl, C₂₋₁₀ heteroalkyl, C₃₋₈ carbocyclic ring, C₃₋₈ heterocyclic ring, deuterated-C₁₋₁₀ alkyl, deuterated-C₂₋₁₀ alkenyl, deuterated-C₂₋₁₀ heteroalkyl, deuterated-C₃₋₈ carbocyclic ring, and deuterated-C₃₋₈ heterocyclic ring optionally fused to an additional optionally substituted carbocyclic, heterocyclic, deuterated-carbocyclic, or deuterated-heterocyclic ring; or R⁵ is a C₁₋₁₀ alkyl, C₂₋₁₀ alkenyl, C₂₋₁₀ heteroalkyl, deuterated-C₁₋₁₀ alkyl, deuterated-C₂₋₁₀ alkenyl, or deuterated-C₂₋₁₀ heteroalkyl substituted with an optionally substituted C₃₋₈ carbocyclic ring, deuterated-C₃₋₈ carbocyclic ring, C₃₋₈ heterocyclic ring, or deuterated-C₃₋₈ heterocyclic ring; and
in each —NR⁴R⁵, R⁴ and R⁵ together with N may form an optionally substituted 3 to 8 membered ring, which may optionally contain an additional heteroatom selected -from N, O and S as a ring member; and the 3 to 8 membered ring optionally contains one or more carbon-bound deuterium;
with the following provisos:
(a) the compound of Formula I, II, III, or IV comprises at least one carbon-bound deuterium; and
(b) when —NR⁴R⁵ in Formula (I) is —NHΦ, where Φ is optionally substituted phenyl:
if all of Z⁵ to Z⁸ are CH or one of Z⁵ to Z⁸ is N, at least one of Z¹ to Z⁴ is CR³ and at least one R³ must be a non-hydrogen substituent; or
if each R³ is H, then Φ must be substituted; or
if all of Z⁵ to Z⁸ are CH or one of Z⁵ to Z⁸ is N, then Z² is not C—OR″, and Z³ is not NH₂, NO₂, NHC(═O)R″ or NHC(═O )—OR″, where R″ is C1-C4 alkyl.

3. The compound of claim 2, wherein at least one of R³ or R⁶ is a polar substituent, wherein said polar substituent is a carboxylic acid, carboxylate salt, carboxylate ester, carboxamide, tetrazole, carboxy bioisostere, deuterated-carboxylic acid, deuterated-carboxylate salt, deuterated-carboxylate ester, deuterated-carboxamide, deuterated-tetrazole, or deuterated-carboxy bioisostere.

4. The compound of claim 2, wherein at least one R³ is a polar substituent.

5. The compound of claim 1, wherein the ring containing Z¹ to Z⁴ is selected from one of the following structures 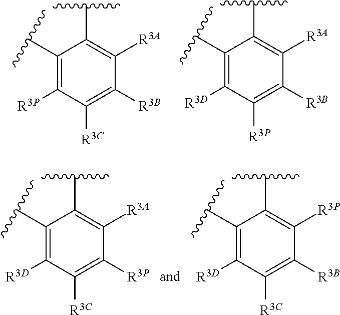
wherein R^{3P} is a polar substituent; and each R^{3A}, R^{3B}, R^{3C} and R^{3D} independently is selected from R³ substituents.

6. The compound of claim 5, wherein each R^{3A}, R^{3C} and R^{3D} is H or deuterium; and R^{3B} is a polar substituent.

7. The compound of claim 1, wherein at least one of Z¹ to Z⁴ and Z⁵ to Z⁸ is a nitrogen atom.

8. The compound of claim 1, wherein R⁴ is H or deuterium.

9. The compound of claim 1, wherein R⁵ is an optionally substituted 3 to 8 membered ring, and the 3 to 8 membered ring optionally contains one or more carbon-bound deuterium.

10. The compound of claim 1, wherein R⁵ is a C₁₋₁₀ alkyl or deuterated-C₁₋₁₀ alkyl group substituted with (1) an optionally substituted 3-8 membered ring, and the 3 to 8 membered ring optionally contains one or more carbon-bound deuterium; or (2) —NR⁴R⁵.

11. The compound of claim 10, wherein R⁵ is a C₁₋₃ alkyl or deuterated-C₁₋₃ alkyl group substituted with (1) an optionally substituted phenyl, pyridyl, morpholino, deuterated-phenyl, deuterated-pyridyl or deuterated-morpholino ring substituent; or (2) substituted with —NR⁴R⁵.

12. The compound of claim 1, wherein R⁵ is an optionally substituted six-membered carbocyclic, heterocyclic, deuterated-carbocyclic, or deuterated-heterocyclic ring.

13. The compound of claim 12, wherein R⁵ is an optionally substituted phenyl or deuterated-phenyl ring.

14. The compound of claim 13, wherein the compound has a structure of Formula I, R⁴ is H, deuterium, CD₃, CHD₂, CH₂D, or CH₃; and R⁵ is a phenyl or deuterated-phenyl substituted with one or more halogen or acetylene substituents.

15. The compound of claim 14, wherein the one or more halogen or acetylene substituents are on the phenyl or deuterated-phenyl ring at the 3-position, 4-position or 5-position, or combinations thereof.

16. The compound of claim 2, wherein the R⁶ substituent is a —NR⁴R⁵ substituent.

17. The compound of claim 16, wherein the R⁶ substituent is a —NH—(C1-C6 alkyl), —ND-(C1-C6 alkyl), —NH-(deuterated-C1-C6 alkyl), —ND-(deuterated-C1-C6 alkyl), —NH—(C3-C8 cycloalkyl), —ND-(C3-C8 cycloalkyl), —NH-(deuterated-C3-C8 cycloalkyl), —ND-(deuterated-C3-C8 cycloalkyl) moiety.

18. The compound of claim 2, having a structural Formulae Ia, Ib, Ic, or Id: 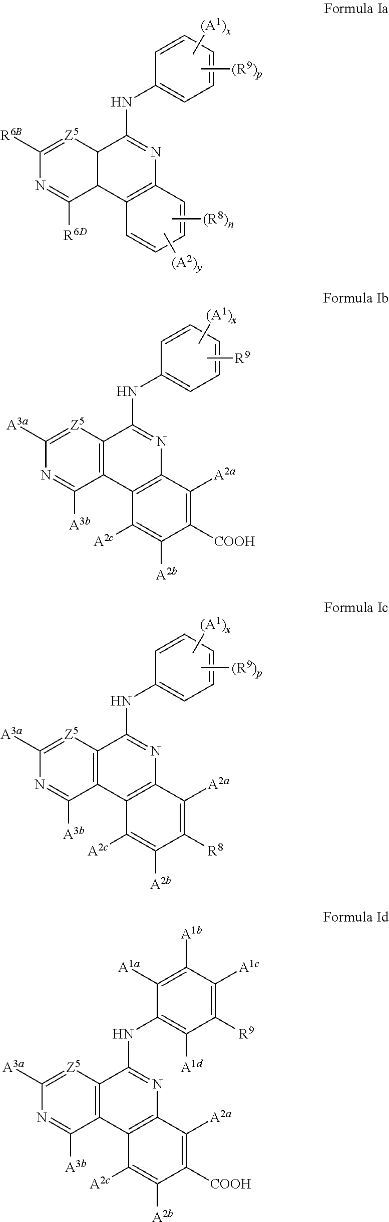
or a pharmaceutically acceptable salt, solvate, and/or prodrug thereof; wherein:
Z⁵ is N or CR^{6A};
each R^{6A}, R^{6B}, R^{6C} and R⁸ independently is H, deuterium, or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C12 heteroaryl, deuterated-C7-C12 arylalkyl, or deuterated-C6-C12 heteroarylalkyl group,
or each R^{6A}, R^{6B}, R^{6C} and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,
each A¹, A^{1a}, A^{1b}, A^{1c}, A^{1d}, A², A^{2a}, A^{2b}, A^{2c}, A^{3a}, and A^{3b} is independently H or deuterium;
R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C12 heteroaryl, deuterated-C7-C12 arylalkyl, or deuterated-C6-C12 heteroarylalkyl group, or
R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H, deuterium, C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, C6-C12 heteroarylalkyl, deuterated-C1-C8 alkyl, deuterated-C2-C8 heteroalkyl, deuterated-C2-C8 alkenyl, deuterated-C2-C8 heteroalkenyl, deuterated-C2-C8 alkynyl, deuterated-C2-C8 heteroalkynyl, deuterated-C1-C8 acyl, deuterated-C2-C8 heteroacyl, deuterated-C6-C10 aryl, deuterated-C5-C10 heteroaryl, deuterated-C7-C12 arylalkyl, or deuterated-C6-C12 heteroarylalkyl;
and wherein two R on the same atom or on adjacent atoms can be linked to form a 3 to 8 membered ring, optionally containing one or more N, O or S; and the 3 to 8 membered ring contains one or more carbon-bound deuterium;
and each R group, and each ring formed by linking two R groups together, is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, deuterium, C1-C6 alkyl, C2-C6 heteroalkyl, C1-C6 acyl, C2-C6 heteroacyl, C6-C10 aryl, C5-C10 heteroaryl, C7-12 arylalkyl, C6-12 heteroarylalkyl, deuterated-C1-C6 alkyl, deuterated-C2-C6 heteroalkyl, deuterated-C1-C6 acyl, deuterated-C2-C6 heteroacyl, deuterated-C6-C10 aryl, deuterated-C5-C10 heteroacyl, deuterated-C7-12 arylalkyl, or deuterated-C6-12 heteroarylalkyl each of which is optionally substituted with one or more groups selected from halo, C1-C4 alkyl, C1-C4 heteroalkyl, C1-C6 acyl, C1-C6 heteroacyl, hydroxy, amino, deuterated-C1-C4 alkyl, deuterated-C1-C4 heteroalkyl, deuterated-C1-C6 acyl, deuterated-C1-C6 heteroacyl, deuterated-hydroxy, deuterated-amino, and ═O;
and wherein two R′ can be linked to form a 3 to 7 membered ring optionally containing up to three heteroatoms selected from N, O and S; and the 3 to 7 membered ring contains one or more carbon-bound deuterium;
x is 1 to 5;
y is 0 to 4;
n is 0 to 4; and
p is 0 to 4; and
with the following provisos:
(a) the compound of Formula Ia, Ib, Ic, or Id comprises at least one carbon-bound deuterium; and
(b) x plus p is 5, and y plus n is 4.

19. The compound of claim 18, wherein Z⁵ is N.

20. The compound of claim 18 or 19, wherein R⁸ is a carboxy moiety, deuterated-carboxy moiety, carboxy bioisostere, or deuterated-carboxy bioisostere.

21. The compound of claim 20, wherein the carboxy or deuterated-carboxy moiety is a carboxylate, deuterated-carboxylate, carboxylic acid, or deuterated-carboxylic acid.

22. The compound of claim 18, wherein R⁹ is selected from —C≡CR, —C≡CH, —C≡CD, methyl, deuterated-methyl, ethyl, deuterated-ethyl, —CF₃, —C≡N, —OR and halogen.

23. The compound of claim 2, having one of the following structures in a deuterated-form:

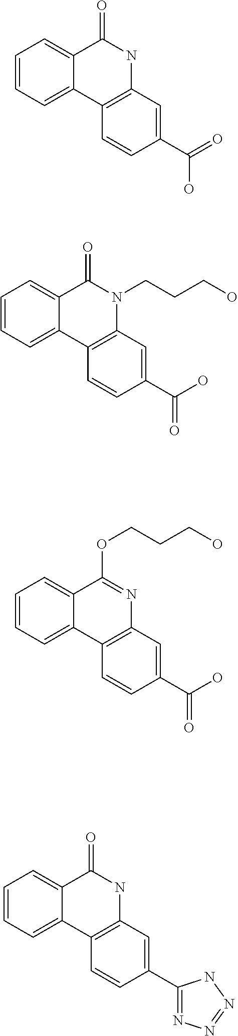

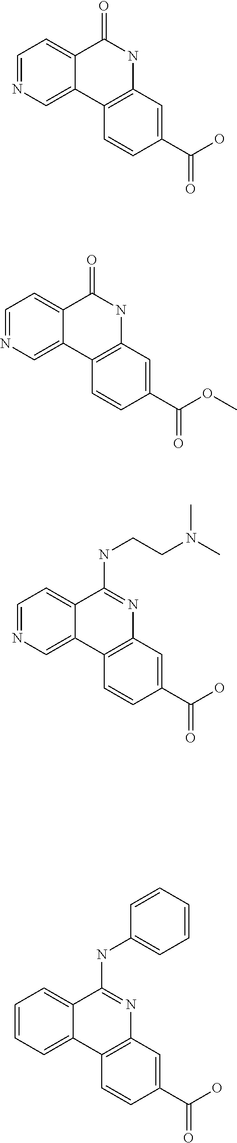

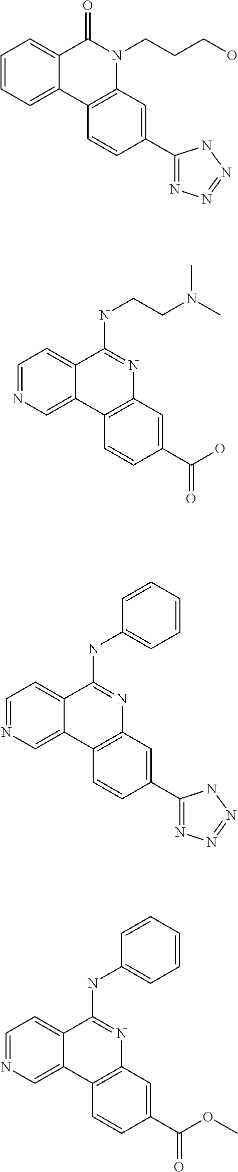

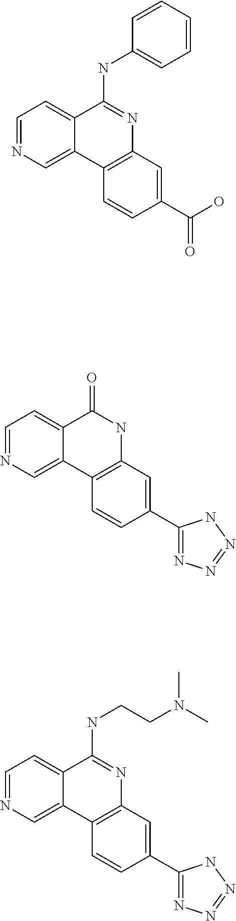

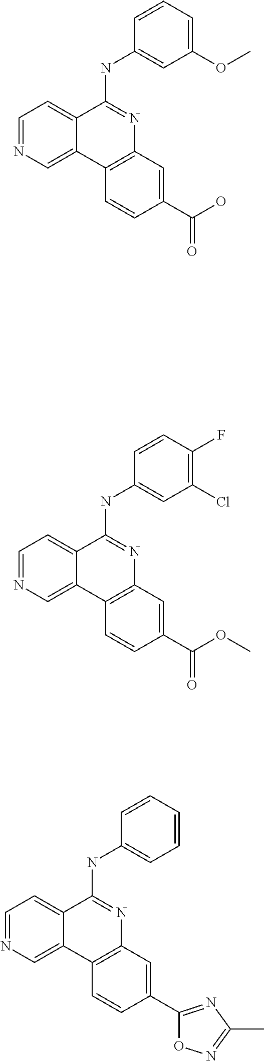

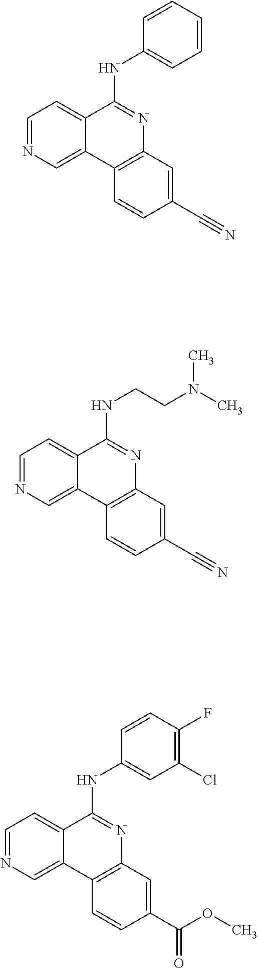

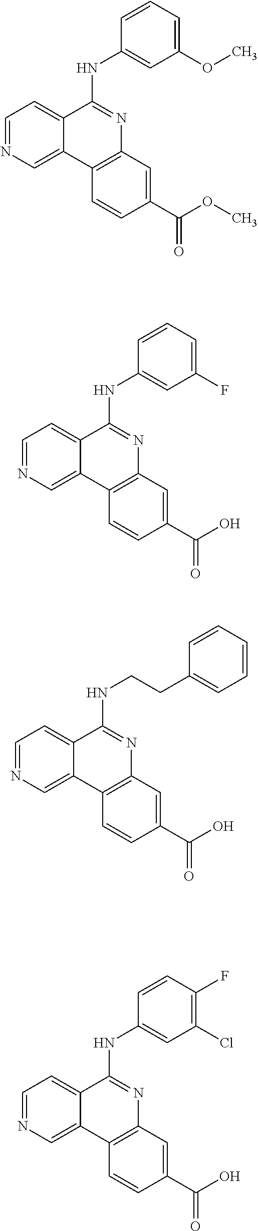

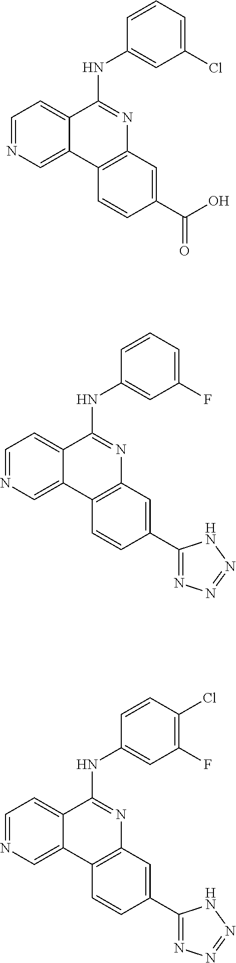

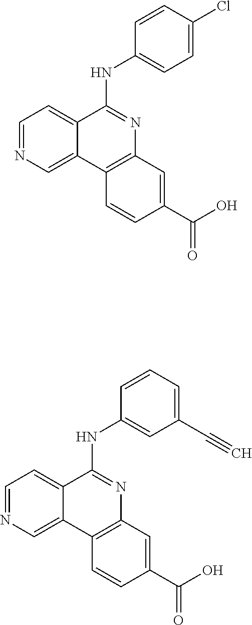

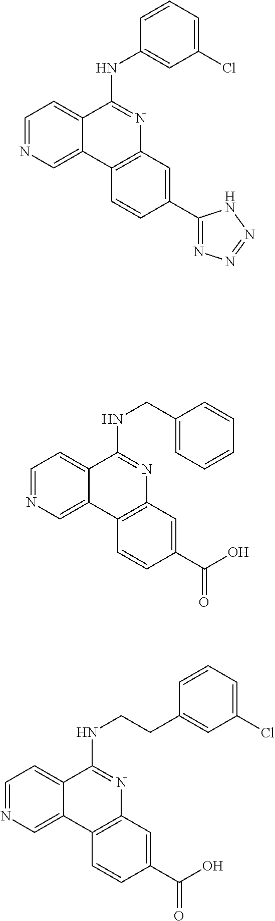

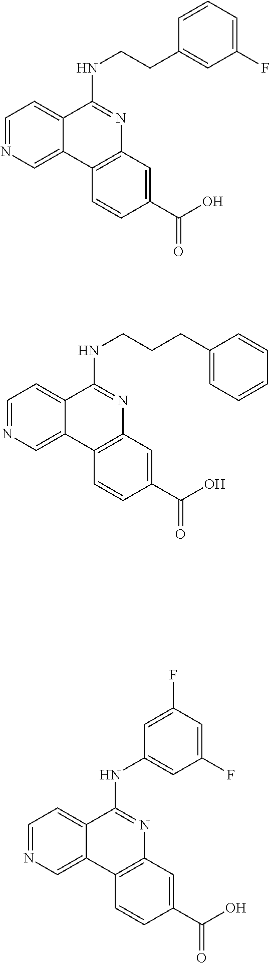

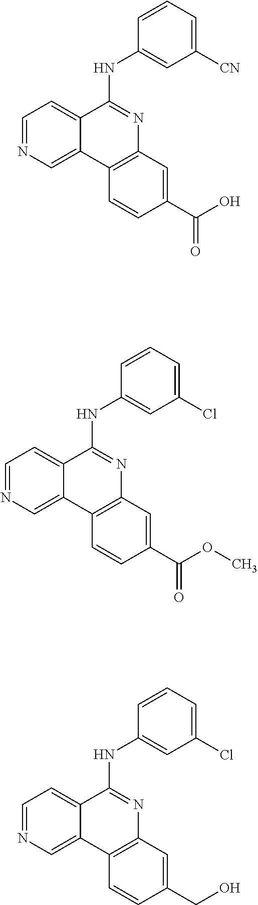

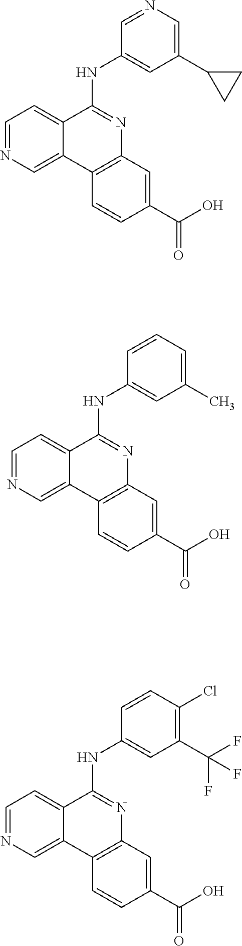

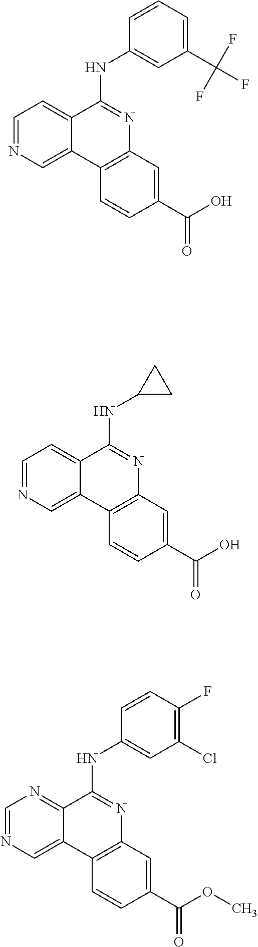

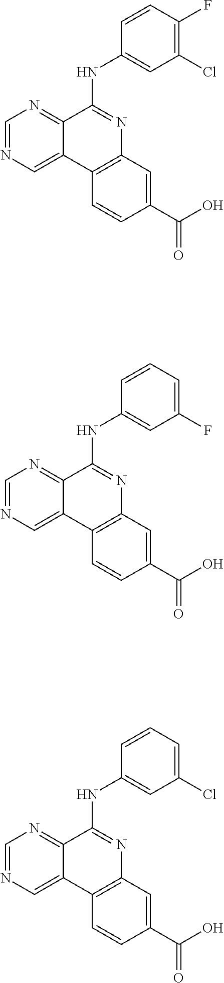

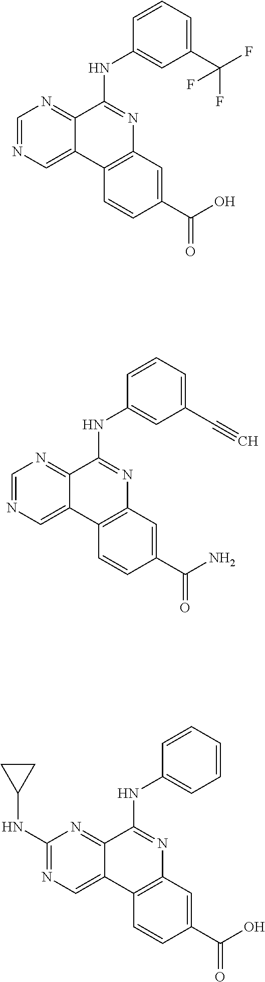

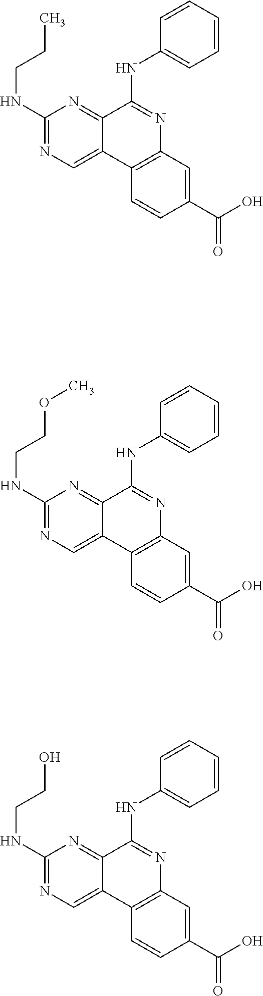

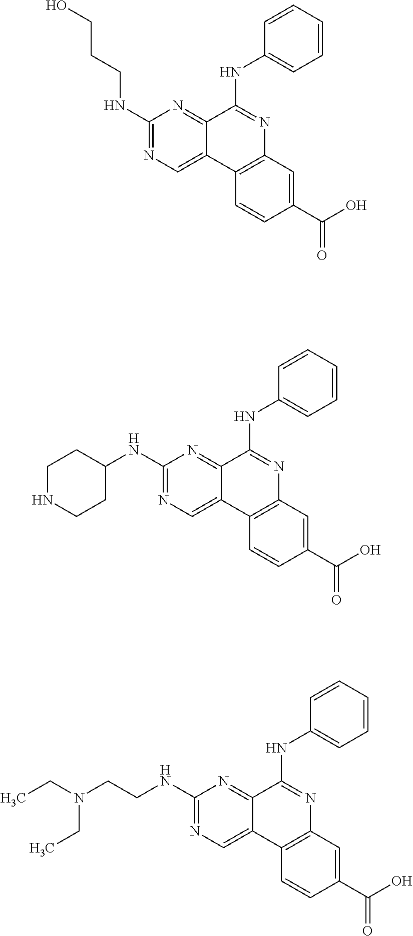

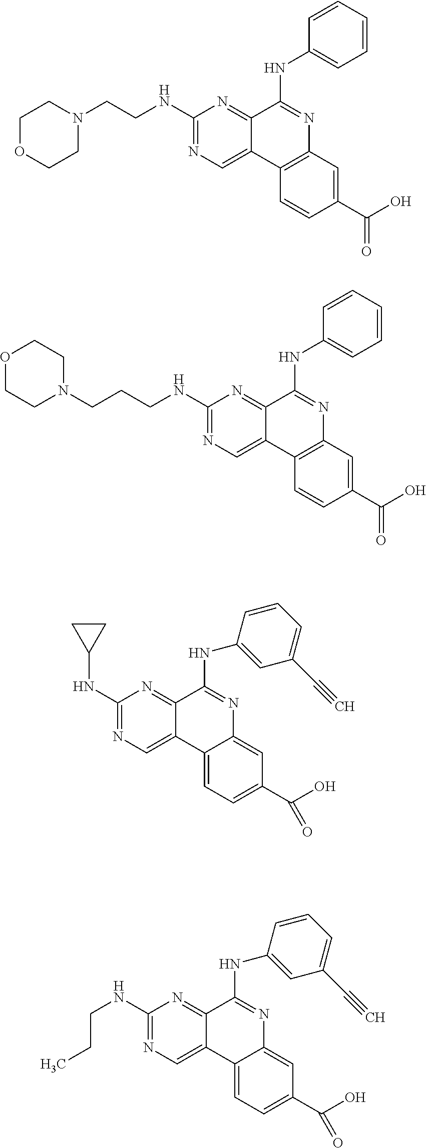

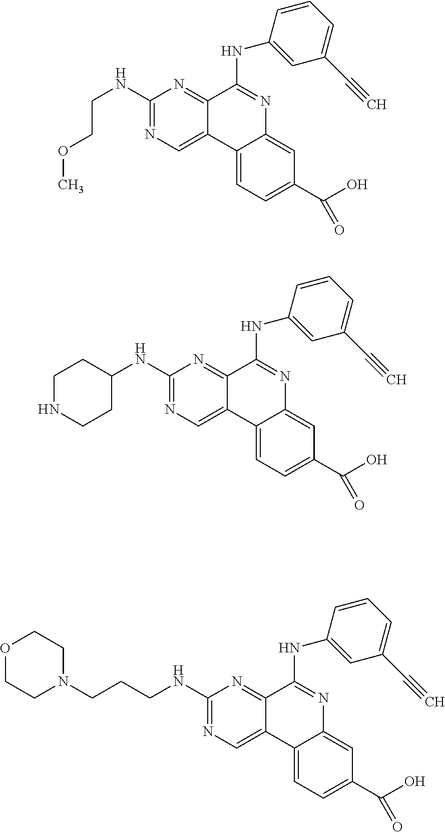

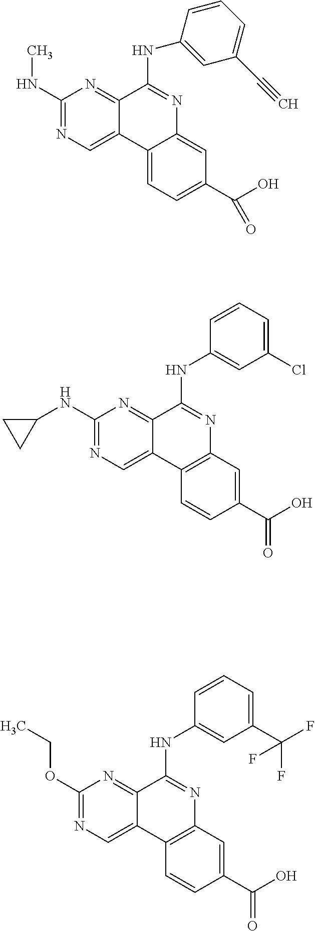

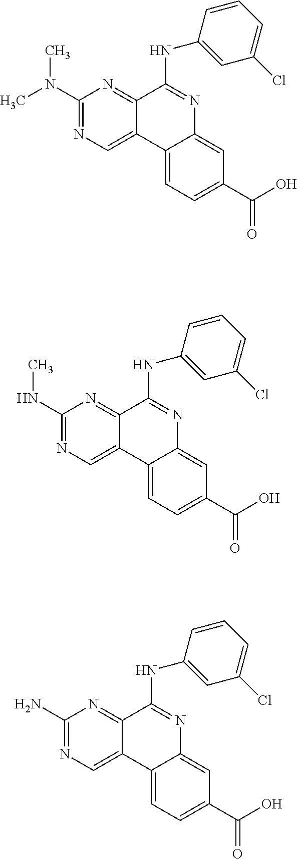

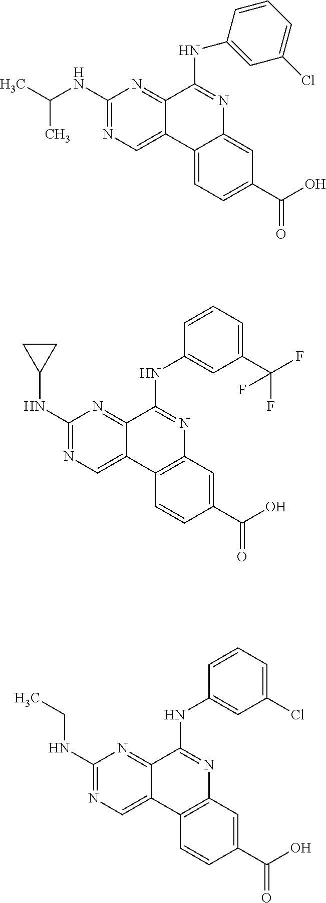

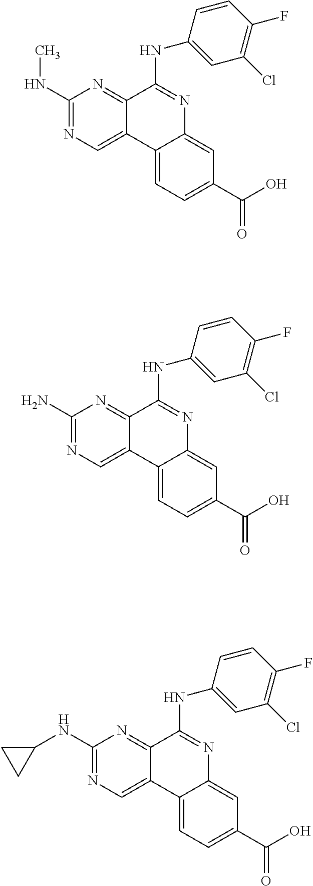

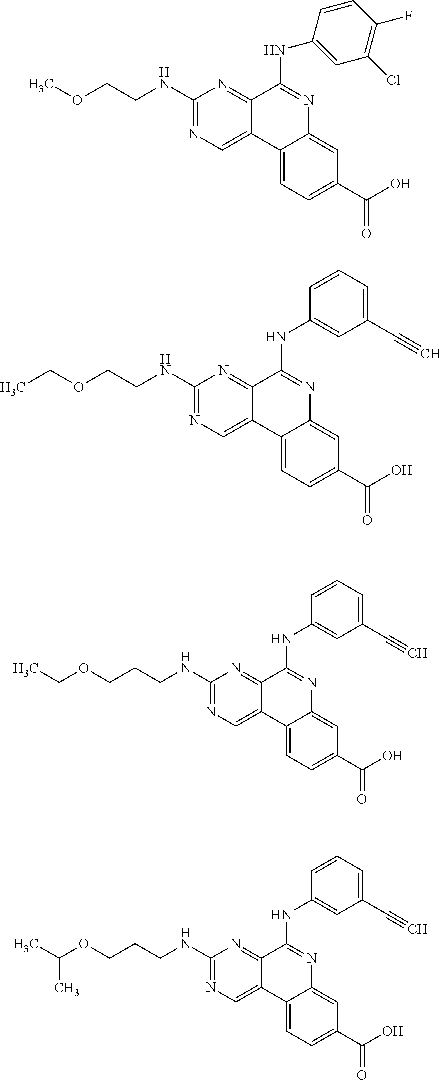

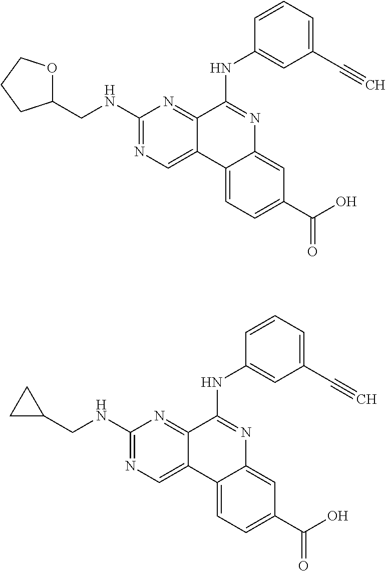

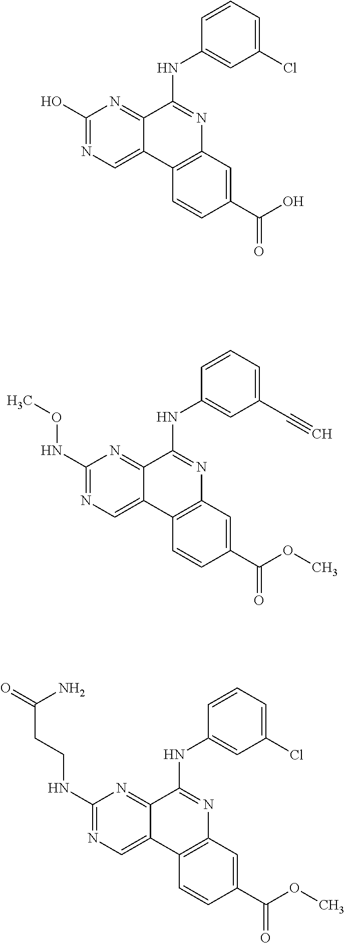

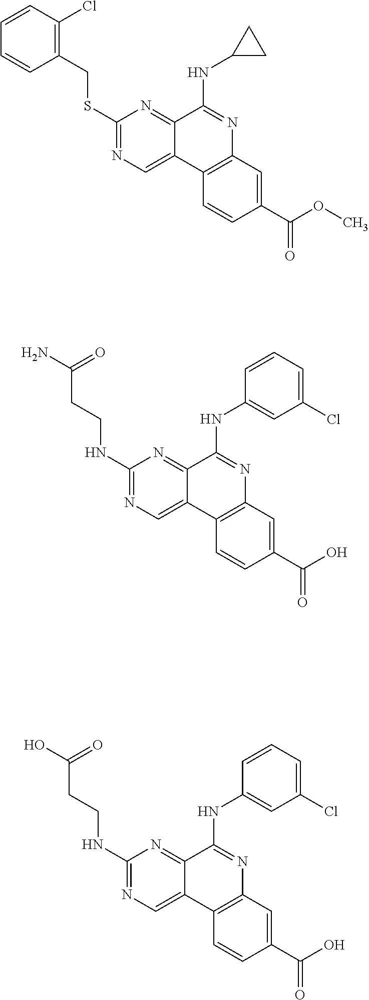

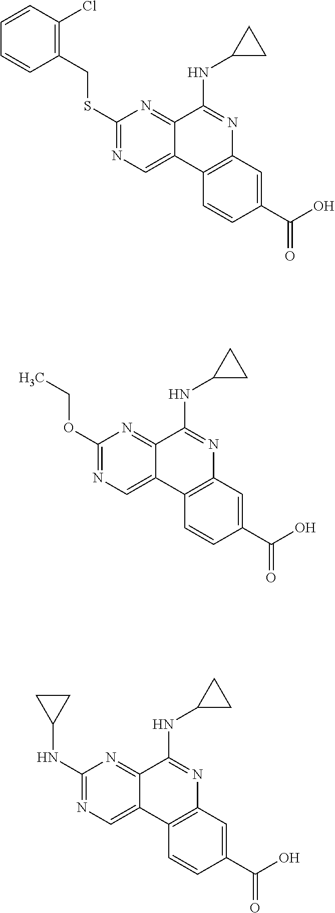

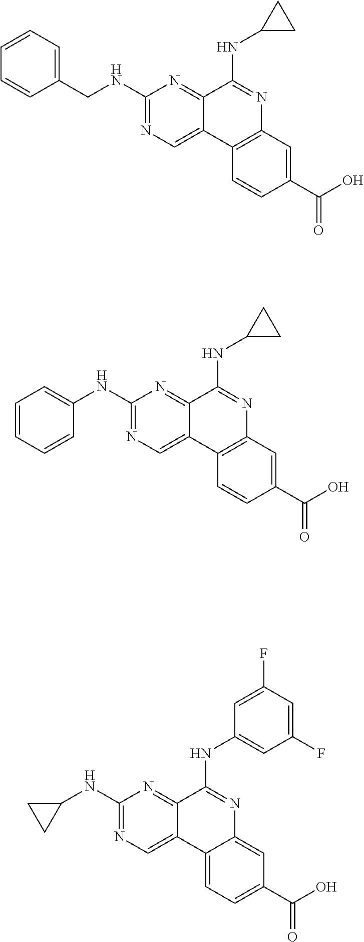

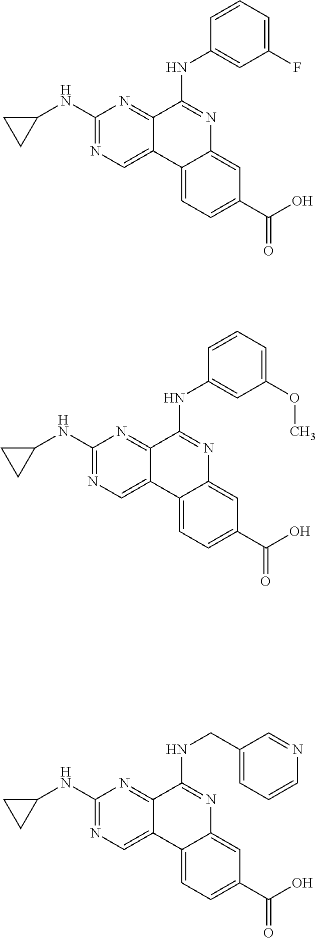

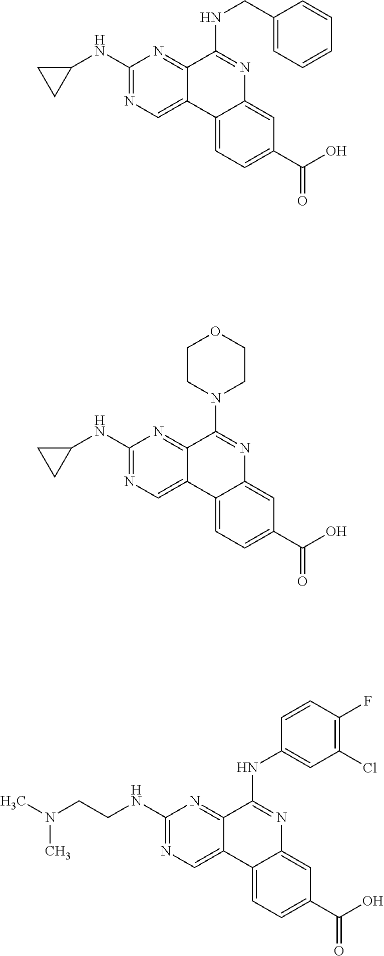

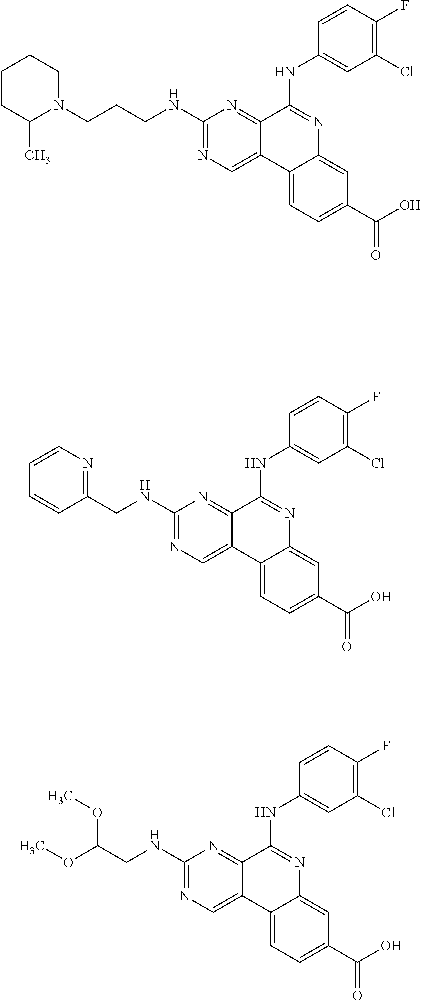

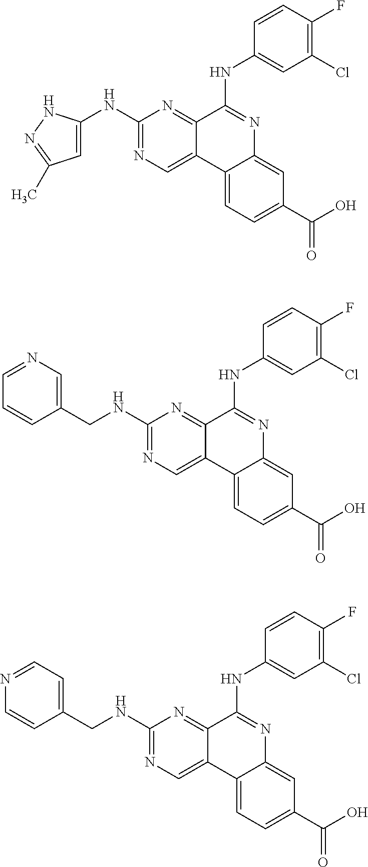

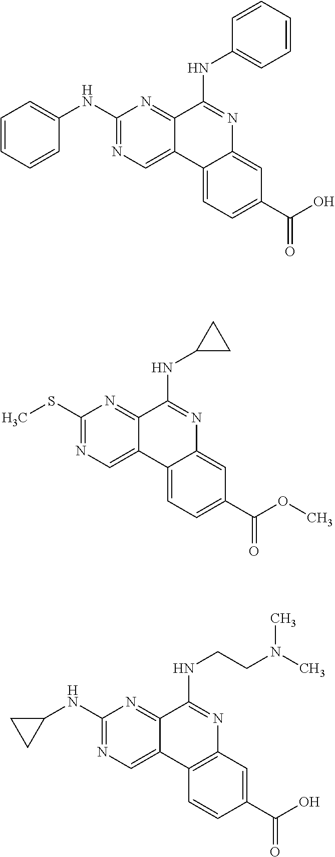

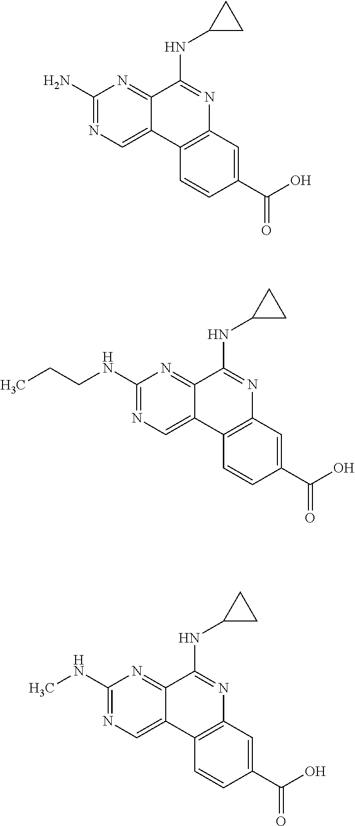

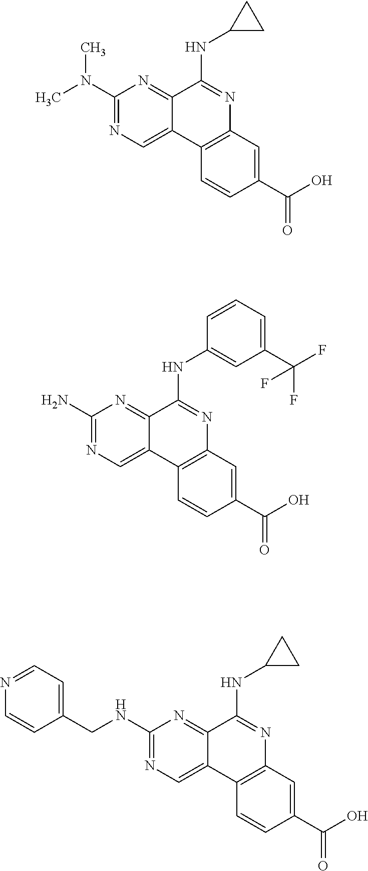

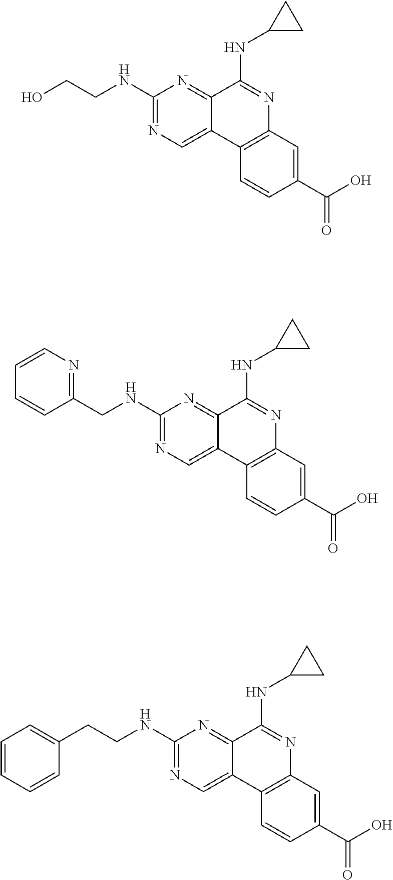

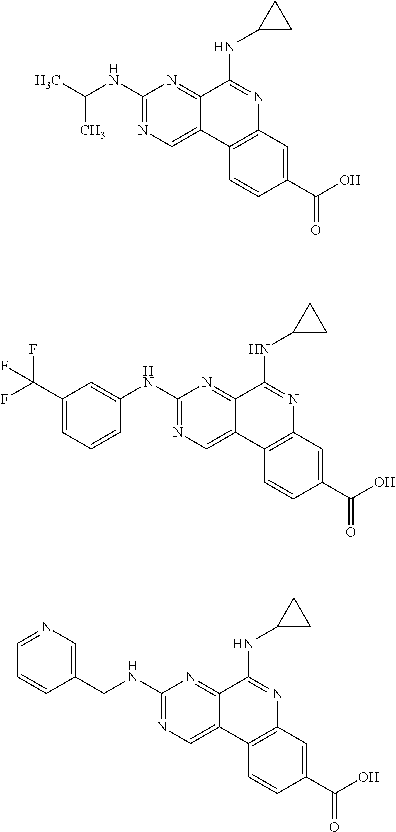

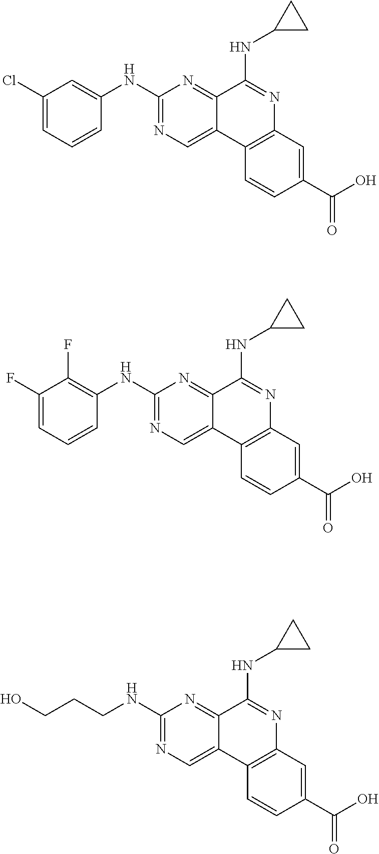

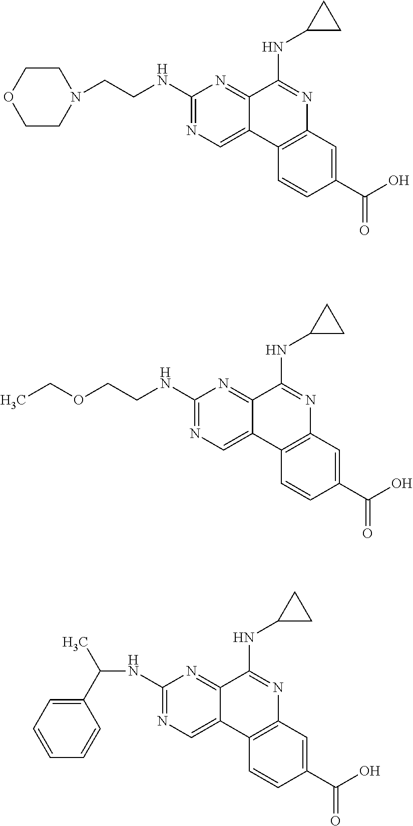

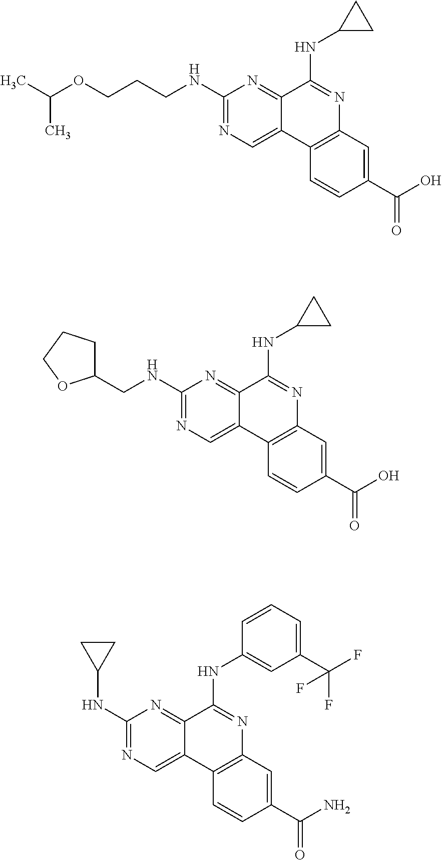

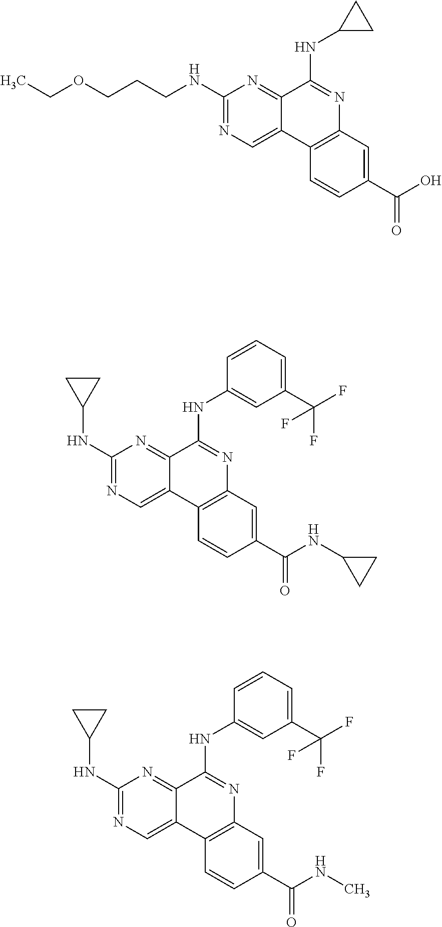

24. The compound of claim 2, having a structural Formula (B1), (B2), or (B3): 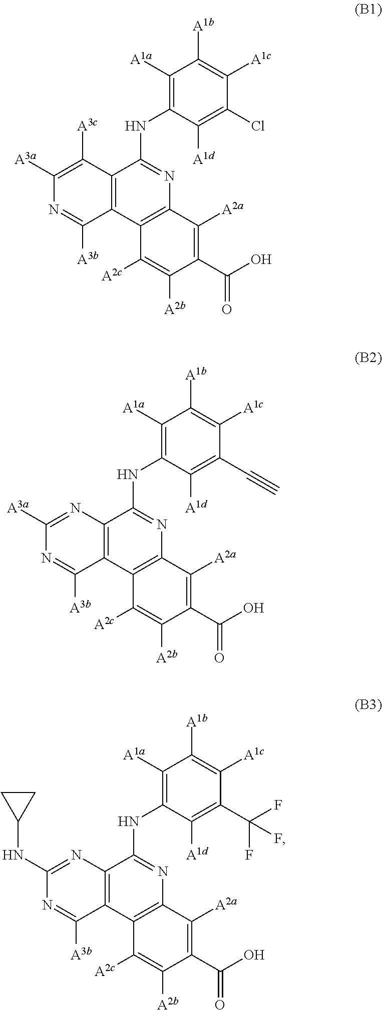
or or a pharmaceutically acceptable salt, solvate, and/or prodrug thereof;
wherein:
each A^{1a}, A^{1b}, A^{1c}, A^{1d}, A^{2a}, A^{2b}, A^{2c}, A^{3a}, A^{3b}, and A^{3c} is independently H or deuterium; and;
with the following provisos:
(a) at least one of A^{1a}, A^{1b}, A^{1c}, A^{1d}, A^{2a}, A^{2b}, A^{2c}, A^{3a}, A^{3b}, and A^{3c} in Formula (B1) is deuterium;
(b) at least one of A^{1a}, A^{1b}, A^{1c}, A^{1d}, A^{2a}, A^{2b}, A^{2c}, A^{3a}, and A^{3b} in Formula (B2) is deuterium; and
(c) at least one of A^{1a}, A^{1b}, A^{1c}, A^{1d}, A^{2a}, A^{2b}, A^{2c}, and A^{3b} in Formula (B3) is deuterium.

25. The compound of claim 24, wherein
each A^{1a}, A^{1b}, A^{2c}, and A^{1d}, is independently H or deuterium;
A^{2a}, A^{2b}, A^{2c}, A^{3a}, A^{3b}, and A^{3c} are H; and
with the proviso that at least one of A^{1a}, A^{1b}, A^{1c}, and A^{1d} is deuterium.

26. The compound of claim 24, wherein
each A^{1a}, A^{1b}, A^{1c}, and A^{1d}, is independently H or deuterium;
each A^{2a}, A^{2b}, and A^{2c} is independently H or deuterium;
A^{3a}, A^{3b}, and A^{3c} are H; and
with the provisos that
(a) at least one of A^{1a}, A^{1b}, A^{1c}, and A^{1d} is deuterium; and
(b) at least one of A^{2a}, A^{2b}, and A^{2c} is deuterium.

27. The compound of claim 24, wherein
each A^{2a}, A^{2b}, and A^{2c} is independently H or deuterium;
A^{1a}, A^{1b, A}^{1c}, A^{1d}, A^{3a}, A^{3b}, and A^{3c} are H; and
with the proviso that at least one of A^{2a}, A^{2b}, and A^{2c} is deuterium.

28. The compound of claim 24, wherein
each A^{3a}, A^{3b}, and A^{3c} is independently H or deuterium;
A^{1a}, A^{1b}, A^{1c}, A^{1d}, A^{2a}, A^{2b}, and A^{2c} are H; and
with the proviso that at least one of A^{3a}, A^{3b}, and A^{3c} is deuterium.

29. A pharmaceutical composition comprising
a compound of claim 1, or a pharmaceutically acceptable salt, solvate, and/or prodrug thereof; and
a pharmaceutically acceptable carrier.

30. A method of modulating a serine-threonine protein kinase activity in a cell, comprising contacting the cell with a compound of claim 1, or a pharmaceutically acceptable salt, solvate, and/or prodrug thereof in an amount effective to modulate a serine-threonine protein kinase activity.

31. A method of inhibiting cell proliferation, comprising contacting cells with a compound of claim 1, or a pharmaceutically acceptable salt, solvate, and/or prodrug thereof in an amount effective to inhibit proliferation of the cells.

32. The method of claim 30, wherein the cells are in a cancer cell line or in a tumor in a subject.

33. A method of treating a condition or disease related to aberrant cell proliferation, comprising administering a therapeutically effective amount of a compound of claim 1, or a pharmaceutically acceptable salt, solvate, and/or prodrug thereof, to a subject in need thereof.

34. The method of claim 33, wherein the condition or disease is a tumor-associated cancer, a non-tumor cancer, or macular degeneration.

35. The method of claim 34, wherein the non-tumor cancer is a hematopoietic cancer.

36. A method of treating a condition or disease associated with a serine-threonine protein kinase activity, comprising administering a therapeutically effective amount of a compound of claim 1, or a pharmaceutically acceptable salt, solvate, and/or prodrug thereof, to a subject in need thereof.

37. The method of claim 30, wherein the serine-threonine protein kinase is casein kinase 2.

38. The method of claim 36, wherein the condition or disease is selected from the group consisting of a cancer, an immunological disorder, a pathogenic infection, an inflammation, a pain, an angiogenesis-related disorder, and combination thereof.

39. The method of claim 38, wherein the condition or disease is a cancer of colorectum, breast, lung, liver, pancreas, lymph node, colon, prostate, brain, head and neck, skin, liver, kidney, or blood and heart.

40. A pharmaceutical composition comprising a compound of claim 1, or a pharmaceutically acceptable salt, solvate, and/or prodrug thereof; and at least one additional therapeutic agent.

41. A method to treat a condition related to aberrant cell proliferation, which comprises co-administering to a subject in need of treatment for such condition a compound of claim 1, or a pharmaceutically acceptable salt, solvate, and/or prodrug thereof; and at least one additional therapeutic agent.
